SEQUENCE LISTING (20 sequences)

SEQ ID NO: 1 - DNA, 5879 bp, Homo sapiens
tcagaagaaagccccgagatcacagagacccggcgagatcacagagacccggcctgaaggaacgtggaaagaccaatgtacctgttttgaccggttgcctggagcaagaagttccagttggggagaattttcagaagataaagtcggagattgtggaaagacttgacttgcagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagataggtgtaacgtggagaaggtgcccagtaattctcagttggaaatagaaggaaacagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 2 - DNA, 5838 bp, Homo sapiens
gcgtcttccggcgcccgcggaggaggcgagggtgggacgctgggcggagcccgagtttaggaagaggaggggacggctgtcatcaatgaagtcatattcataatctagtcctctctccctctgtttctgtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 3 - DNA, 5664 bp, Homo sapiens
gcgtcttccggcgcccgcggaggaggcgagggtgggacgctgggcggagcccgagtttaggaagaggaggggacggctgtcatcaatgaagtcatattcataatctagtcctctctccctctgtttctgtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 4 - DNA, 1846 bp, Homo sapiens
cttggagaggcggaggtggaaacgatgcgcaggagttggcttggggctttttgtttgcgtgtccctgtttacctattcataatcatggatcccctctgctttgtgatactgtgaaccacgcataacagcaattctttacaccaccgggttgagaagaaggcgcctgaggctgactttctggacctgccgtcacgcagtaaagatgtggttggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 5 - protein, 1590 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 6 - protein, 1572 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 7 - protein, 1514 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 8 - protein, 248 aa, Homo sapiens
MWLAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 9 - DNA, 5589 bp, Homo sapiens
ggcgggggaggcgccgggaagtcgacggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaaaattacaggaaaagatatccatgacacagccagcagtgtggcacgtgggctacaattccagcgtggccttcagttctgcacacgtgcgtcaaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 10 - DNA, 5779 bp, Homo sapiens
ggagaggccgccgcggtgctgagggggaggggagccggcgagcgcgcgcgcagcgggggcgcgggtggcgcgcgtgtgtgtgaagggggggcggtggccgaggcgggcgggcgcgcgcgcgaggcttcccctcgtttggcggcggcggcggcttctttgtttcgtgaagagaagcgagacgcccattctgcccccggccccgcgcggaggggcgggggaggcgccgggaagtcgacggcgccggcggctcctgcgtctcgcccttttgcccaggctagagtgcagtggtgcggtcatggttcactgcagcctcaacctcctggactcagcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 11 - DNA, 5329 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaccctgaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 12 - DNA, 5326 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaccctgaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 13 - DNA, 5320 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 14 - DNA, 5317 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 15 - protein, 1679 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKKITGKDIHDTASSVARGLQFQRGLQFCTRASKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 16 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 17 - protein, 1617 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKTLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 18 - protein, 1616 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKTLKAIEEGTLEEIEEEVRQKKSSRKRKRDSDAGSSTPTTSTRSRDKDDESKKQKKRGRPPAEKLSPNPPNLTKKMKKIVDAVIKYKDSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 19 - protein, 1614 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 20 - protein, 1613 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

(19) United States
(12) Patent Application Publication
(10) Pub. No.: US 2023/0000849 A1
(43) Pub. Date: Jan. 5, 2023

(54) INHIBITION OF SMARCA2 FOR TREATMENT OF CANCER

(71) Applicant: Epizyme, Inc., Cambridge, MA (US)

(72) Inventors: Alexandra Rose GRASSIAN, Cambridge, MA (US); Allison DREW, Arlington, MA (US)

(21) Appl. No.: 17/848,885

(22) Filed: Jun. 24, 2022

Related U.S. Application Data
(63) Continuation of application No. 16/489,489, filed on Aug. 28, 2019, now abandoned.
(60) Provisional application No. 62/464,811, filed on Feb. 28, 2017.
(60) Provisional application No. 62/542,241, filed on Aug. 7, 2017.

(51) Int. Cl. A61K 31/4439; A61K 31/4709; A61K 31/4745; A61K 31/551; G01N 33/574; A61K 31/713

(52) U.S. Cl. CPC A61K 31/4439; A61K 31/4709; A61K 31/4745; A61K 31/551; G01N 33/57484; A61K 31/713; C12N 2310/20

(57) ABSTRACT

The present disclosure provides treatment modalities, e.g., strategies, treatment methods, patient stratification methods, combinations, and compositions that are useful for the treatment of disorders, e.g., proliferative disorders, such as certain cancer. Some aspects of this disclosure provide treatment modalities, methods, strategies, compositions, combinations, and dosage forms for the treatment of cell proliferative disorders, e.g., cancers with decreased activity or function, or loss of function, of SMARCA4 with a SMARCA2 antagonist.

### RELATED APPLICATIONS

This application is a continuation of U.S. application Ser. No. 16/489,489, filed Aug. 28, 2019, which is a U.S. National Phase application, filed under 35 U.S.C. § 371, of International Application No. PCT/US2018/020124, filed Feb. 28, 2018, which claims the benefit of and priority to U.S. Provisional Application Nos. 62/464,811, filed Feb. 28, 2017, and 62,542,241, filed Aug. 7, 2017, the entire contents of each of which are hereby incorporated by reference.

### INCORPORATION BY REFERENCE OF SEQUENCE LISTING

The contents of the text file named “EPIZ-077C01US_ST25.txt”, which was created on Jun. 24, 2022, and is about 197,024 bytes in size, are incorporated herein by reference in their entireties.

### FIELD OF DISCLOSURE

This disclosure relates to modulation (e.g., inhibition) of SMARCA2 for treating cancer.

### SUMMARY

The present disclosure provides treatment modalities, e.g., strategies, treatment methods, patient stratification methods, combinations, and compositions that are useful for the treatment of disorders, e.g., proliferative disorders, such as certain cancers. Some aspects of this disclosure provide treatment modalities, methods, strategies, compositions, combinations, and dosage forms for the treatment of cell proliferative disorders, e.g., cancers, associated with a certain biomarker, or patient stratification methods based on detection of a biomarker.

Some aspects of this disclosure provide methods comprising modulating (e.g., inhibiting) a SMARCA2 activity in a cell exhibiting a decreased activity or function of SMARCA4 (e.g., a loss of function of SMARCA4).

Some aspects of this disclosure provide methods of treating cancer in a subject in need thereof, comprising administering a therapeutically effective amount of a SMARCA2 antagonist to the subject or a cell of the subject. In some embodiments, the subject or cell of the subject exhibits a decreased activity or function of SMARCA4 when compared to a control level of the activity or the function of SMARCA4.

Some aspects of the disclosure relate to a SMARCA2 antagonist for use in the treatment of cancer in a cell or subject, wherein the cell or subject exhibits decreased activity or function of SMARCA4 when compared to a control level of the activity or the function of SMARCA4.

Some aspects of the disclosure relate to a SMARCA2 antagonist for use as a medicament for the treatment of cancer in a cell or subject, wherein the cell or subject exhibits decreased activity or function of SMARCA4 when compared to a control level of the activity or the function of SMARCA4.

Some aspects of the disclosure relate to the use of a SMARCA2 antagonist in the manufacture of a medicament for the treatment of cancer in a cell or subject, wherein the cell or subject exhibits decreased activity or function of SMARCA4 when compared to a control level of the activity or the function of SMARCA4.

Some aspects of this disclosure provide methods of inhibiting an activity of SMARCA2, comprising contacting SMARCA2 enzyme with a SMARCA2 antagonist. In some embodiments, the SMARCA2 enzyme is within a cell, e.g., a cancer cell, and the method comprises contacting the cell with the SMARCA2 inhibitor, wherein the cell comprises a biomarker of sensitivity to the SMARCA2 antagonist.

Some aspects of this disclosure provide a SMARCA2 antagonist for use in inhibiting an activity of SMARCA2, wherein the SMARCA2 antagonist is contacted with a SMARCA2 enzyme. In some embodiments, the SMARCA2 enzyme is within a cell, e.g., a cancer cell, wherein the cell comprises a biomarker of sensitivity to the SMARCA2 antagonist.

Some aspects of this disclosure provide a SMARCA2 antagonist for use as a medicament for inhibiting an activity of SMARCA2, wherein the medicament is contacted with a SMARCA2 enzyme. In some embodiments, the SMARCA2 enzyme is within a cell, e.g., a cancer cell, wherein the cell comprises a biomarker of sensitivity to the SMARCA2 antagonist.

Some aspects of this disclosure provide the use of a SMARCA2 antagonist in the manufacture of a medicament for inhibiting an activity of SMARCA2, wherein the medicament is to be contacted with a SMARCA2 enzyme. In some embodiments, the SMARCA2 enzyme is within a cell, e.g., a cancer cell, wherein the cell comprises a biomarker of sensitivity to the SMARCA2 antagonist.

Some aspects of this disclosure provide methods of treating cancer in a subject in need thereof, comprising administering to the subject a therapeutically effective amount of a SMARCA2 antagonist, wherein the subject or a cell of the subject comprises a biomarker of sensitivity to the SMARCA2 antagonist.

Some aspects of this disclosure provide a SMARCA2 antagonist for use in treating cancer in a subject in need thereof, wherein the subject or a cell of the subject comprises a biomarker of sensitivity to the SMARCA2 antagonist.

Some aspects of this disclosure provide a SMARCA2 antagonist for use as a medicament for treating cancer in a subject in need thereof, wherein the subject or a cell of the subject comprises a biomarker of sensitivity to the SMARCA2 antagonist.

Some aspects of this disclosure provide the use of a SMARCA2 antagonist in the manufacture of a medicament for treating cancer in a subject in need thereof, wherein the subject or a cell of the subject comprises a biomarker of sensitivity to the SMARCA2 antagonist.

In some embodiments, the biomarker is a decreased activity or function of SMARCA4. In certain embodiments, the biomarker is loss of function of SMARCA4.

Some aspects of this disclosure provide methods of identifying a subject sensitive to treatment with a SMARCA2 antagonist, comprising detecting a decreased activity or function of SMARCA4 compared to a control level of the activity or the function of SMARCA4 in the subject and administering the SMARCA2 antagonist to the subject, wherein the subject has a cancer and wherein an improvement in a sign or symptom of the cancer indicates a sensitivity of the subject or of a cancer cell of the subject for the SMARCA2 antagonist.

In some embodiments, the control level is the level of activity of SMARCA4 in a subject that does not have cancer.

In some embodiments, the subject is a participant in a clinical trial. In some embodiments, a criterion for participation of a subject in the clinical trial is a decreased activity or function of SMARCA4, or loss of function of SMARCA4, in said subject or a cell of said subject.

In some embodiments, the present disclosure features a method comprising inhibiting a SMARCA2 activity in a cell exhibiting loss of function of SMARCA4.

In certain embodiments of the methods disclosed herein, the SMARCA2 activity is an ATPase activity.

In certain embodiments of the methods, uses, or medicaments disclosed herein, the SMARCA2 activity is not a bromodomain activity.

In some embodiments, the methods of the disclosure comprise contacting a cell with a SMARCA2 antagonist. In certain embodiments, the cell is in vivo, ex vivo, in vitro, or in situ. In certain embodiments of the methods disclosed herein, the cell is in a subject.

In some embodiments, the cell is ex vivo or in vitro. In further embodiments, the cell is isolated or derived from a subject that has a tumor.

In some embodiments, the tumor is malignant. In some embodiments, the tumor is metastatic.

In some embodiments, the methods of the disclosure comprise administering a SMARCA2 antagonist to a subject.

In some embodiments of the disclosure, the SMARCA2 antagonist does not modulate SMARCA4. For example, the SMARCA2 antagonist does not inhibit SMARCA4.

In some embodiments of the disclosure, the SMARCA2 antagonist targets a helicase domain of SMARCA2.

In some embodiments of the disclosure, the SMARCA2 antagonist targets an ATPase domain of SMARCA2.

In some embodiments of the disclosure, the SMARCA2 antagonist does not target a bromodomain activity of SMARCA2.

In some embodiments of the disclosure, the decreased activity of SMARCA4 is caused by a genetic mutation.

In some embodiments of the disclosure, the decreased activity of SMARCA4 is caused by an epigenetic alteration.

In some embodiments of the disclosure, the decreased activity of SMARCA4 is caused by a decrease in SMARCA4 gene transcription, SMARCA4 gene transcript translation, or a combination thereof.

In some embodiments of the disclosure, the SMARCA2 antagonist is selected from the group consisting of antisense RNA, shRNA, siRNA, CRISPR/Cas9, transcription activator-like effector nucleases (TALEN), Zinc Finger nucleases (ZFN), antibodies, antibody fragments and antibody mimetics.

In some embodiments, the SMARCA2 antagonist is a SMARCA2 inhibitor. In certain embodiments, the SMARCA2 inhibitor is a selective SMARCA2 inhibitor.

In certain embodiments of the methods disclosed herein, the cell is in a subject, and the method comprises administering a SMARCA2 inhibitor to the subject.

In certain embodiments of the disclosure, the SMARCA2 inhibitor inhibits an ATPase activity of SMARCA2.

In certain embodiments of the disclosure, the SMARCA2 inhibitor selectively inhibits an ATPase activity of SMARCA2.

In some aspects, this present disclosure features methods of treating cancer, comprising inhibiting a SMARCA2 activity in a subject in need thereof, wherein the subject has a cancer characterized by loss of function of SMARCA4.

In some embodiments, the SMARCA2 antagonist is a SMARCA2 inhibitor. In some embodiments, the SMARCA2 inhibitor is selected from the group consisting of BMCL 2968, I-BET151, JQ1, and PFI-3. In some embodiments, the SMARCA2 inhibitor is PFI-3.

In some aspects, this present disclosure features methods of treating cancer, comprising inhibiting a SMARCA2 activity, e.g., a SMARCA2 helicase activity or a SMARCA2 ATPase activity, in a subject in need thereof, wherein the subject has a cancer characterized by loss of function of SMARCA4.

Some aspects of this disclosure provide methods comprising modulating a SMARCA2 activity in a cell exhibiting a decreased activity or function of SMARCA4. In some embodiments, the cell is in vivo, ex vivo, in vitro, or in situ. In some embodiments, the cell is in a subject, and the method comprises administering a SMARCA2 antagonist to the subject. In some embodiments, the cell is ex vivo or in vitro, and wherein the cell is isolated or derived from a subject that has a tumor. In some embodiments, the tumor is malignant. In some embodiments, the tumor is metastatic.

Some aspects of this disclosure provide methods of treating cancer in a subject in need thereof, comprising administering a therapeutically effective amount of a SMARCA2 antagonist to the subject or a cell of the subject, wherein said subject or cell of the subject exhibits a decreased activity or function of SMARCA4 when compared to a control level of the activity or the function of SMARCA4.

Some aspects of this disclosure provide a SMARCA2 antagonist for use in treating cancer in a subject in need thereof, wherein said subject or a cell of the subject exhibits a decreased activity or function of SMARCA4 when compared to a control level of the activity or the function of SMARCA4.

Some aspects of this disclosure provide a SMARCA2 antagonist for use as a medicament for treating cancer in a subject in need thereof, wherein said subject or a cell of the subject exhibits a decreased activity or function of SMARCA4 when compared to a control level of the activity or the function of SMARCA4.

Some aspects of this disclosure provide the use of a SMARCA2 antagonist in the manufacture of a medicament for treating cancer in a subject in need thereof, wherein said subject or a cell of the subject exhibits a decreased activity or function of SMARCA4 when compared to a control level of the activity or the function of SMARCA4.

In some embodiments, the control level is the level of activity or function of SMARCA4 in a subject that does not have cancer. In some embodiments, the method comprises administering the SMARCA2 antagonist to the cell or the subject based on the decreased activity or function of SMARCA4 in the cell or the subject.

Some aspects of this disclosure provide methods of identifying a subject having a cancer as a candidate for treatment with a SMARCA2 antagonist, comprising detecting a level of activity or function of SMARCA4 in a cancer cell in the subject, comparing the level of activity or function of SMARCA4 detected in the cancer cell to a control or reference level, wherein the subject is identified as a candidate for treatment with a SMARCA2 antagonist, if the level of activity or function of SMARCA4 in the cancer cell is decreased as compared to the control or reference level. In some embodiments, the method comprises obtaining a sample comprising a cancer cell from the subject.

Some aspects of this disclosure provide methods of identifying a cancer cell as sensitive to treatment with a SMARCA2 antagonist, comprising detecting a level of activity or function of SMARCA4 in the cancer cell, comparing the level of activity or function of SMARCA4 detected in the cancer to a control or reference level, wherein the cell is identified as sensitive to treatment with a SMARCA2 antagonist, if the level of activity or function of SMARCA4 is decreased as compared to the control or reference level. In some embodiments, the control or reference level of SMARCA4 activity or function is a level of SMARCA4 observed or expected in a healthy cell of the same origin as the cancer cell.

In some embodiments, the SMARCA2 antagonist inhibits SMARCA2 helicase activity by at least 10%, at least 20%, at least 30%, at least 40%, at least 50%, at least 60%, at least 70%, at least 80%, at least 90%, at least 95%, at least 98%, or at least 99%, or abolishes SMARCA2 activity. In some embodiments, the SMARCA2 antagonist inhibits SMARCA2 ATPase activity by at least 10%, at least 20%, at least 30%, at least 40%, at least 50%, at least 60%, at least 70%, at least 80%, at least 90%, at least 95%, at least 98%, or at least 99%, or abolishes SMARCA2 activity. In some embodiments, the SMARCA2 antagonist is a selective SMARCA2 antagonist. In some embodiments, the SMARCA2 antagonist inhibits SMARCA2 activity at least 2-fold, at least 5-fold, at least 10-fold, at least 20-fold, at least 50-fold, at least 100-fold, at least 1000-fold, at least 10000-fold, or at least 100000-fold more efficiently than SMARC4 activity. In some embodiments, the SMARCA2 antagonist does not inhibit SMARCA4.

In some embodiments, the SMARCA2 antagonist targets a helicase domain of SMARCA2. In some embodiments, the SMARCA2 antagonist targets an ATPase domain of SMARCA2. In some embodiments, the SMARCA2 antagonist does not target a bromodomain activity of SMARCA2.

In some embodiments, the decreased activity of SMARCA4 is caused by a genetic mutation. In some embodiments, the decreased activity of SMARCA4 is caused by an epigenetic alteration. In some embodiments, the decreased activity of SMARCA4 is caused by a decrease in SMARCA4 gene transcription, by a decrease in SMARCA4 gene transcript translation, by a post-translational modification, by a loss of protein-protein interaction, or a combination thereof.

In some embodiments, the SMARCA2 antagonist is a small molecule SMARCA2 inhibitor. In some embodiments, the SMARCA2 antagonist is selected from the group consisting of antisense RNA, shRNA, siRNA, CRISPR/Cas9, transcription activator-like effector nucleases (TALEN), Zinc Finger nucleases (ZFN), antibodies, antibody fragments and antibody mimetics.

Any of the above aspects and embodiments can be combined with any other aspect or embodiment.

Other features and advantages of the invention will be apparent from the following drawings, detailed description, and claims.

### BRIEF DESCRIPTIONS OF FIGURES

The patent or application file contains at least one drawing executed in color. Copies of this patent or patent application publication with color drawing(s) will be provided by the Office upon request and payment of the necessary fee.

The above and further features will be more clearly appreciated from the following detailed description when taken in conjunction with the accompanying drawings.

FIG. 1 is a graph showing CRISPR pooled screen data, illustrating sensitivity (Log P RSA) to SMARCA2 knockout. Cell lines are colored by SMARCA4 expression: blue represents high SMARCA4 expression, red represents low SMARCA4 expression. Cell lines which are sensitive to SMARCA2 knockout tend to have low SMARCA4 expression.

FIG. 2 is a graph showing a transcriptomic analysis of NSCLC cell lines that have RNA seq. data available in Cancer Cell Line Encyclopedia (CCLE). The Figure demonstrates that only cell lines with low SMARCA4 expression are sensitive to SMARCA2 knockout.

FIGS. 3A-3E are a series of images of immunohistochemistry (IHC) slides of non-small cell lung cancer tumor samples, screened for SMARCA2/4 protein expression. FIGS. 3A-3E show samples with protein expression as follows: FIG. 3A: double negative sample (loss of SMARCA2 and SMARCA4); FIG. 3B: SMARCA4 negative sample; FIG. 3C: SMARCA2 negative sample; FIG. 3D: wild type samples; FIG. 3E: double positive sample (SMARCA2 and SMARCA4 expression present).

FIG. 4 is a graph validating the anti-proliferative effect of SMARCA2 knockout in SMARCA4 mutant cell lines. The figure shows the percent change in target CRISPR cells lines over time following infection with the viral delivery vector for the CRISPR construct in SMARCA4 mutant cell lines.

FIG. 5 is a graph demonstrating that inhibition of the ATPase domain drives antiproliferative effects in cells. The graph shows the antiproliferative effect of SMARCA2 knockout as a function of CRISPR guide target.

FIGS. 6A and 6B are a series of graphs illustrating antiproliferative effects of bromodomain inhibitor PFI-3. FIG. 6A shows that PFI-3 binds to SMACA2 with nanomolar affinity. FIG. 6B shows that PFI-3 does not impact cell growth in SMARCA4-wt or mutant cell lines.

FIGS. 7A-7G are a series of graphs demonstrating that isolated full length SMARCA2 is well behaved in activity assays. FIG. 7A summarizes the signal to background ratio (S:B) in an ATPase high throughput bioluminescence assay. The S:B ratio was found to remain linear for 90 minutes, with a value of 10 at 5 nM of SMARCA2. FIG. 7B is a plot of luminescence as a function of SMARCA2 concentration. FIG. 7C is a plot showing the results of a biosubstrate analysis. The value of KM was determined as 640 uM and 5.8 mM for ATP and mononucleosome, respectively. FIG. 7D illustrates DMSO tolerance. FIG. 7E illustrates uniformity of the assay. The z-factor was determined to 0.70. FIGS. 7F and 7G illustrate the determination of IC₅₀ values for reference inhibitors.

FIGS. 8A-8F are a series of graphs demonstrating behavior of SMARCA4 in an activity assay. FIG. 8A summarizes the signal to background ratio (S:B) in an ATPase high throughput bioluminescence assay. The S:B ratio was found to remain linear for 90 minutes, with a value of 7 at 5 nM of SMARCA4. FIG. 8B is a plot of luminescence as a function of SMARCA4 concentration. FIG. 8C is a plot showing the results of a biosubstrate analysis for ATP. The value of KM was determined as 133 mM. FIG. 8D is a plot showing the results of a biosubstrate analysis for mononucleosome. The value of KM was determined as 2.1 mM. FIG. 8E illustrates uniformity of the assay. The z-factor was determined to 0.71. FIG. 8F illustrates the determination of IC₅₀ values for a reference inhibitor.

FIGS. 9A and 9B are a series of graphs illustrating the behavior of purified SWI/SNF complex in an ATPase assay. FIG. 9A is an illustration of SWI/SNF complex purification from HEK293 cells using a SMARCB-1 flag. FIG. 9B shows the ATPase activity as a function of concentration for the purified complex.

FIGS. 10A-D are a series of graphs illustrating that the purified SWI/SNF protein complex demonstrates similar kinetic parameters to SMARCA2. FIG. 10A is a plot of SWI/SNF and SMARCA2 activity as a function of mononucleosome concentration. FIG. 10B is a plot of SWI/SNF and SMARCA2 activity as a function of ATP concentration. FIG. 10C is a plot of ATP levels as a function of time for various concentrations of the SWI/SNF protein complex. FIG. 10D is a plot of luminescence as a function of the SWI/SNF protein complex concentration.

FIGS. 11A-11C illustrate the detection and validation of a small molecule SMARCA2 ATPase inhibitor (ADP). FIG. 11A is a plot of surface plasmon resonance response of the binding affinity of the SMARCA2 inhibitor to truncated SMARCA2 as a function of time. FIG. 11B is a plot of surface plasmon resonance response of the binding affinity of the SMARCA2 inhibitor to truncated SMARCA2 as a function of inhibitor concentration. The K_{d} value was determined as 7 μM. FIG. 11C is a plot of ATPase inhibition in full length (FL) and truncated (TR) SMARCA2, measured using a 2-amino-6-mercapto-7-methylpurine ribonucleoside/Purine Nucleoside Phosphorylase (MESG/PNP) assay. The IC₅₀ values of the SMARCA2 inhibitor were determined as 28 μM and 23 μM, for FL-SMARCA2 and TR-SMARCA2 IC₅₀, respectively.

FIG. 12 is a Western Blot Analysis for SMARCA4 and SMARCA2 for various non-small cell lung cancer cell lines.

### DETAILED DESCRIPTION

The present disclosure provides treatment modalities, methods, strategies, compositions, combinations, and dosage forms for the treatment of cell proliferative disorders, e.g., cancers, associated with decreased activity or function of SMARCA4 (e.g., loss of function of SMARCA4). Some aspects of this disclosure provide patient stratification methods based on detection of a decreased activity or function, or loss of function, of SMARCA4.

In some aspects, this present disclosure features methods comprising modulating a SMARCA2 activity in a cell exhibiting a decreased activity or function of SMARCA4 (e.g., loss of function of SMARCA4).

In some aspects, this present disclosure features methods of treating cancer in a subject in need thereof, comprising administering a therapeutically effective amount of a SMARCA2 antagonist to the subject or a cell of the subject.

In some aspects, the present disclosure features a SMARCA2 antagonist for use in the treatment of cancer in a subject in need thereof.

In some aspects, the present disclosure features a SMARCA2 antagonist for use as a medicament for the treatment of cancer in a subject in need thereof.

In some aspects, the present disclosure features the use of a SMARCA2 antagonist in the manufacture of a medicament for the treatment of cancer in a subject in need thereof.

In some embodiments, the subject or cell of the subject exhibits a decreased activity or function of SMARCA4 compared to a control level of the activity or the function of SMARCA4.

In some aspects, this present disclosure features methods of modulating an activity of SMARCA2, comprising contacting a cell with a SMARCA2 antagonist, wherein the cell comprises a biomarker of sensitivity to SMARCA2 inhibition.

In some aspects, the present disclosure features a SMARCA2 antagonist for use in modulating an activity of SMARCA2, wherein said use comprises contacting a cell with a SMARCA2 antagonist, wherein the cell comprises a biomarker of sensitivity to SMARCA2 inhibition.

In some aspects, the present disclosure features a SMARCA2 antagonist as a medicament for modulating an activity of SMARCA2, wherein said medicament is for contacting with a cell, wherein the cell comprises a biomarker of sensitivity to SMARCA2 inhibition.

In some aspects, the present disclosure features the use of a SMARCA2 antagonist in the manufacture of a medicament for modulating an activity of SMARCA2, wherein said medicament is for contacting with a cell, wherein the cell comprises a biomarker of sensitivity to SMARCA2 inhibition.

In some aspects, this present disclosure features methods of treating cancer in a subject in need thereof, comprising administering to the subject a therapeutically effective amount of a SMARCA2 antagonist, wherein the subject or a cell of the subject comprises a biomarker of sensitivity to the SMARCA2 antagonist.

In some aspects, the present disclosure features a SMARCA2 antagonist for use in the treatment of cancer in a subject in need thereof, wherein the subject or a cell of the subject comprises a biomarker of sensitivity to the SMARCA2 antagonist.

In some aspects, the present disclosure features a SMARCA2 antagonist for use as a medicament for the treatment of cancer in a subject in need thereof, wherein the subject or a cell of the subject comprises a biomarker of sensitivity to the SMARCA2 antagonist.

In some aspects, the present disclosure features the use of a SMARCA2 antagonist in the manufacture of a medicament for the treatment of cancer in a subject in need thereof, wherein the subject or a cell of the subject comprises a biomarker of sensitivity to the SMARCA2 antagonist.

In some embodiments, the biomarker is a decreased activity or function of SMARCA4. In certain embodiments, the biomarker is loss of function of SMARCA4.

In some aspects, this present disclosure features methods of identifying a subject sensitive to treatment with a SMARCA2 antagonist, comprising detecting a decreased activity or function of SMARCA4 compared to a control level of the activity or the function of SMARCA4 in the subject and administering the SMARCA2 antagonist to the subject, wherein the subject has a cancer and wherein an improvement in a sign or symptom of the cancer indicates a sensitivity of the subject or of a cancer cell of the subject for the SMARCA2 antagonist.

In some embodiments, the subject is a participant in a clinical trial. In some embodiments, a criterion for participation of a subject in the clinical trial is a decreased activity or function of SMARCA4, or loss of function of SMARCA4, in said subject or a cell of said subject.

In some embodiments, the control level is the level of activity of SMARCA4 in a subject that does not have cancer.

In some embodiments, the present disclosure features a method comprising inhibiting a SMARCA2 activity in a cell exhibiting loss of function of SMARCA4.

In certain embodiments of the methods disclosed herein, the SMARCA2 activity is an ATPase activity.

In certain embodiments of the methods disclosed herein, the SMARCA2 activity is not a bromodomain activity.

In some embodiments, the methods of the disclosure comprise contacting a cell with a SMARCA2 antagonist. In certain embodiments, the cell is in vivo, ex vivo, in vitro, or in situ. In certain embodiments of the methods disclosed herein, the cell is in a subject.

In some embodiments, the cell is ex vivo or in vitro. In further embodiments, the cell is isolated or derived from a subject that has a tumor.

In some embodiments, the tumor is malignant. In some embodiments, the tumor is metastatic.

In some embodiments, the methods of the disclosure comprise administering a SMARCA2 antagonist to the subject.

In some embodiments of the disclosure, the SMARCA2 antagonist does not modulate SMARCA4. For example, the SMARCA2 antagonist does not inhibit SMARCA4.

In some embodiments of the disclosure, the SMARCA2 antagonist targets a helicase domain of SMARCA2.

In some embodiments of the disclosure, the SMARCA2 antagonist targets an ATPase domain of SMARCA2.

In some embodiments of the disclosure, the SMARCA2 antagonist does not target a bromodomain activity of SMARCA2.

In some embodiments of the disclosure, the decreased activity of SMARCA4 is caused by a genetic mutation.

In some embodiments of the disclosure, the decreased activity of SMARCA4 is caused by an epigenetic process, e.g., silencing of a SMARCA4 gene, post-transcriptional or post-translational modulation of the half-life of a SMARCA4 gene product, e.g., inhibition of translation of a SMARCA4 transcript into SMARCA4 protein, or increased turnover of a SMARCA4 protein.

In some embodiments of the disclosure, the decreased activity of SMARCA4 is caused by a decrease in SMARCA4 gene transcription, SMARCA4 gene transcript translation, or a combination thereof.

In some embodiments of the disclosure, the SMARCA2 antagonist is selected from the group consisting of antisense RNA, shRNA, siRNA, CRISPR/Cas9, transcription activator-like effector nucleases (TALEN), Zinc Finger nucleases (ZFN), antibodies, antibody fragments and antibody mimetics.

In some embodiments, the SMARCA2 antagonist is a small molecule SMARCA2 inhibitor (e.g., ADP). In certain embodiments, the SMARCA2 inhibitor is a selective SMARCA2 inhibitor, e.g., in that it inhibits SMARCA2, but not SMARCA4 or a different helicase, or in that it inhibits SMARCA2 more efficiently than SMARCA4.

In certain embodiments of the methods disclosed herein, the cell is in a subject, and the method comprises administering a SMARCA2 inhibitor to the subject.

In certain embodiments of the methods disclosed herein, the SMARCA2 inhibitor inhibits an ATPase activity of SMARCA2.

In certain embodiments of the methods disclosed herein, the SMARCA2 inhibitor selectively inhibits an ATPase activity of SMARCA2.

Some aspects of this disclosure provide methods of treating cancer, comprising inhibiting a SMARCA2 activity in a subject in need thereof, wherein the subject has a cancer characterized by loss of function of SMARCA4.

In some embodiments, the SMARCA2 antagonist is a SMARCA2 inhibitor. In some embodiments, the SMARCA2 inhibitor is selected from the group consisting of BMCL 2968, I-BET151, JQ1, and PFI-3. In some embodiments, the SMARCA2 inhibitor is PFI-3.

Some aspects of this disclosure provide methods of treating cancer, comprising inhibiting a SMARCA2 activity, e.g., a SMARCA2 helicase activity or a SMARCA2 ATPase activity, in a subject in need thereof, wherein the subject has a cancer characterized by loss of function of SMARCA4.

### SMARCA2/SMARCA4

Some aspects of this disclosure are based on the recognition that SMARCA2 is a synthetic lethal target in SMARCA4-mutated cancers or cancers associated with decrease or loss of activity or a function of SMARCA4. Some aspects of this disclosure thus provide methods or medicaments for decreasing or abolishing survival and/or proliferation of cancer cells that exhibit a loss of SMARCA4 function by inhibiting SMARCA2 in such cells.

SMARCA2 and SMARCA4 are SWI/SNF related, matrix associated, actin dependent regulators of chromatin and mutually exclusive paralogs in the SWI/SNF complex. SWI/SNF complexes regulate many cell processes by direct modulation of nucleosomal structure. The catalytic subunits SMARCA2 and SMARCA4 have ATP-dependent helicase activity that repositions nucleosomes.

SWI/SNF complex members are mutated in about 20% of human cancers (Kardoch et al. Nat. Genet., 2013, 45(6), 592-601, incorporated herein by reference in its entirety). For example SMARCA4 mutations occur across a diverse range of cancer types with varying population size and clinical need.

Table 1 below provides a summary of the frequency of SMARCA4 mutations in certain cancer types.

TABLE 1
SMARCA4 mutations in certain cancers

| | | | | Estimated |
|---|---|---|---|---|
| | SMARCA4 | | 5 Year | SMARCA4- |
| | Mutations | US | Survival | Mutant |
| Cancer Type | (%) | Cases/Year | (%) | Patients/Year |
| Ovary - | >95% | <300 | 33% | <300 |
| SCCOHT | | | | |
| Bladder | 8% | 75,000 | 77% | 6000 |
| Stomach | 6% | 22,000 | 28% | 1320 |
| Lung | 4-5% | 220,000 | 17% | ~10,000 |
| | (NSCLC) | | | |
| Glioma/GBM | 2-5% | 20,000 | Variable | ~360 |
| Head and | 4% | 36,000 | 56% | 1440 |
| Neck | | | | |
| Kidney | 3-4% (Clear | 64,000 | 72% | ~2000 |
| | cell, | | | |
| | Papillary) | | | |
| Uterine/ | 3-4% | 12,000 | 68% | ~400 |
| Cervical | | | | |
| Pancreas | 3% | 46,000 | 7% | 1380 |

However, SMARCA4 expression can also be regulated by post-transcriptional and post-translational mechanisms. As such, an analysis of mutation frequencies only is likely to underestimate protein loss, and observing only mutations of SMARCA4 may underestimate decrease or loss of activity or a function of SMARCA4 in a patient. Decrease or loss of activity or a function of SMARCA4 can appear in patients who have not mutation of SMARCA4. These patients can by identified by methods such as mRNA or protein assays. In some embodiments of the present disclosure, methods comprising detecting a loss of activity or function of SMARCA4 in a cell or tissue comprise assaying SMARCA4 protein expression levels by a suitable method, such as, e.g., antibody-based assays allowing for quantification of expressed protein in the cell or tissue (e.g., western blot, immunohistochemistry, ELISA, etc.).

Exemplary sequences for SMACA2 and SMARCA4 are provided below:

SMARCA2
mRNA sequence of human SWI/SNF related, matrix associated, actin
dependent regulator of chromatin, subfamily a, member 2 (SMARCA2),
transcript variant 3 (GenBank Accession No. NM_001289396.1)
(SEQ ID NO: 1)
TCAGAAGAAAGCCCCGAGATCACAGAGACCCGGCGAGATCACAGAGACCCGGCCTGAAGGAACGTGGAAA
GACCAATGTACCTGTTTTGACCGGTTGCCTGGAGCAAGAAGTTCCAGTTGGGGAGAATTTTCAGAAGATA
AAGTCGGAGATTGTGGAAAGACTTGACTTGCAGCATTACTCTACTGACTGGCAGAGACAGGAGAGGTAGA
TGTCCACGCCCACAGACCCTGGTGCGATGCCCCACCCAGGGCCTTCGCCGGGGCCTGGGCCTTCCCCTGG
GCCAATTCTTGGGCCTAGTCCAGGACCAGGACCATCCCCAGGTTCCGTCCACAGCATGATGGGGCCAAGT
CCTGGACCTCCAAGTGTCTCCCATCCTATGCCGACGATGGGGTCCACAGACTTCCCACAGGAAGGCATGC
ATCAAATGCATAAGCCCATCGATGGTATACATGACAAGGGGATTGTAGAAGACATCCATTGTGGATCCAT
GAAGGGCACTGGTATGCGACCACCTCACCCAGGCATGGGCCCTCCCCAGAGTCCAATGGATCAACACAGC
CAAGGTTATATGTCACCACACCCATCTCCATTAGGAGCCCCAGAGCACGTCTCCAGCCCTATGTCTGGAG
GAGGCCCAACTCCACCTCAGATGCCACCAAGCCAGCCGGGGGCCCTCATCCCAGGTGATCCGCAGGCCAT
GAGCCAGCCCAACAGAGGTCCCTCACCTTTCAGTCCTGTCCAGCTGCATCAGCTTCGAGCTCAGATTTTA
GCTTATAAAATGCTGGCCCGAGGCCAGCCCCTCCCCGAAACGCTGCAGCTTGCAGTCCAGGGGAAAAGGA
CGTTGCCTGGCTTGCAGCAACAACAGCAGCAGCAACAGCAGCAGCAGCAGCAGCAGCAGCAGCAGCAGCA
GCAGCAACAGCAGCCGCAGCAGCAGCCGCCGCAACCACAGACGCAGCAACAACAGCAGCCGGCCCTTGTT
AACTACAACAGACCATCTGGCCCGGGGCCGGAGCTGAGCGGCCCGAGCACCCCGCAGAAGCTGCCGGTGC
CCGCGCCCGGCGGCCGGCCCTCGCCCGCGCCCCCCGCAGCCGCGCAGCCGCCCGCGGCCGCAGTGCCCGG
GCCCTCAGTGCCGCAGCCGGCCCCGGGGCAGCCCTCGCCCGTCCTCCAGCTGCAGCAGAAGCAGAGCCGC
ATCAGCCCCATCCAGAAACCGCAAGGCCTGGACCCCGTGGAAATTCTGCAAGAGCGGGAATACAGACTTC
AGGCCCGCATAGCTCATAGGATACAAGAACTGGAAAATCTGCCTGGCTCTTTGCCACCAGATTTAAGAAC
CAAAGCAACCGTGGAACTAAAAGCACTTCGGTTACTCAATTTCCAGCGTCAGCTGAGACAGGAGGTGGTG
GCCTGCATGCGCAGGGACACGACCCTGGAGACGGCTCTCAACTCCAAAGCATACAAACGGAGCAAGCGCC
AGACTCTGAGAGAAGCTCGCATGACCGAGAAGCTGGAGAAGCAGCAGAAGATTGAGCAGGAGAGGAAACG
CCGTCAGAAACACCAGGAATACCTGAACAGTATTTTGCAACATGCAAAAGATTTTAAGGAATATCATCGG
TCTGTGGCCGGAAAGATCCAGAAGCTCTCCAAAGCAGTGGCAACTTGGCATGCCAACACTGAAAGAGAGC
AGAAGAAGGAGACAGAGCGGATTGAAAAGGAGAGAATGCGGCGACTGATGGCTGAAGATGAGGAGGGTTA
TAGAAAACTGATTGATCAAAAGAAAGACAGGCGTTTAGCTTACCTTTTGCAGCAGACCGATGAGTATGTA
GCCAATCTGACCAATCTGGTTTGGGAGCACAAGCAAGCCCAGGCAGCCAAAGAGAAGAAGAAGAGGAGGA
GGAGGAAGAAGAAGGCTGAGGAGAATGCAGAGGGTGGGGAGTCTGCCCTGGGACCGGATGGAGAGCCCAT
AGATGAGAGCAGCCAGATGAGTGACCTCCCTGTCAAAGTGACTCACACAGAAACCGGCAAGGTTCTGTTC
GGACCAGAAGCACCCAAAGCAAGTCAGCTGGACGCCTGGCTGGAAATGAATCCTGGTTATGAAGTTGCCC
CTAGATCTGACAGTGAAGAGAGTGATTCTGATTATGAGGAAGAGGATGAGGAAGAAGAGTCCAGTAGGCA
GGAAACCGAAGAGAAAATACTCCTGGATCCAAATAGCGAAGAAGTTTCTGAGAAGGATGCTAAGCAGATC
ATTGAGACAGCTAAGCAAGACGTGGATGATGAATACAGCATGCAGTACAGTGCCAGGGGCTCCCAGTCCT
ACTACACCGTGGCTCATGCCATCTCGGAGAGGGTGGAGAAACAGTCTGCCCTCCTAATTAATGGGACCCT
AAAGCATTACCAGCTCCAGGGCCTGGAATGGATGGTTTCCCTGTATAATAACAACTTGAACGGAATCTTA
GCCGATGAAATGGGGCTTGGAAAGACCATACAGACCATTGCACTCATCACTTATCTGATGGAGCACAAAA
GACTCAATGGCCCCTATCTCATCATTGTTCCCCTTTCGACTCTATCTAACTGGACATATGAATTTGACAA
ATGGGCTCCTTCTGTGGTGAAGATTTCTTACAAGGGTACTCCTGCCATGCGTCGCTCCCTTGTCCCCCAG
CTACGGAGTGGCAAATTCAATGTCCTCTTGACTACTTATGAGTATATTATAAAAGACAAGCACATTCTTG
CAAAGATTCGGTGGAAATACATGATAGTGGACGAAGGCCACCGAATGAAGAATCACCACTGCAAGCTGAC
TCAGGTCTTGAACACTCACTATGTGGCCCCCAGAAGGATCCTCTTGACTGGGACCCCGCTGCAGAATAAG
CTCCCTGAACTCTGGGCCCTCCTCAACTTCCTCCTCCCAACAATTTTTAAGAGCTGCAGCACATTTGAAC
AATGGTTCAATGCTCCATTTGCCATGACTGGTGAAAGGGTGGACTTAAATGAAGAAGAAACTATATTGAT
CATCAGGCGTCTACATAAGGTGTTAAGACCATTTTTACTAAGGAGACTGAAGAAAGAAGTTGAATCCCAG
CTTCCCGAAAAAGTGGAATATGTGATCAAGTGTGACATGTCAGCTCTGCAGAAGATTCTGTATCGCCATA
TGCAAGCCAAGGGGATCCTTCTCACAGATGGTTCTGAGAAAGATAAGAAGGGGAAAGGAGGTGCTAAGAC
ACTTATGAACACTATTATGCAGTTGAGAAAAATCTGCAACCACCCATATATGTTTCAGCACATTGAGGAA
TCCTTTGCTGAACACCTAGGCTATTCAAATGGGGTCATCAATGGGGCTGAACTGTATCGGGCCTCAGGGA
AGTTTGAGCTGCTTGATCGTATTCTGCCAAAATTGAGAGCGACTAATCACCGAGTGCTGCTTTTCTGCCA
GATGACATCTCTCATGACCATCATGGAGGATTATTTTGCTTTTCGGAACTTCCTTTACCTACGCCTTGAT
GGCACCACCAAGTCTGAAGATCGTGCTGCTTTGCTGAAGAAATTCAATGAACCTGGATCCCAGTATTTCA
TTTTCTTGCTGAGCACAAGAGCTGGTGGCCTGGGCTTAAATCTTCAGGCAGCTGATACAGTGGTCATCTT
TGACAGCGACTGGAATCCTCATCAGGATCTGCAGGCCCAAGACCGAGCTCACCGCATCGGGCAGCAGAAC
GAGGTCCGGGTACTGAGGCTCTGTACCGTGAACAGCGTGGAGGAAAAGATCCTCGCGGCCGCAAAATACA
AGCTGAACGTGGATCAGAAAGTGATCCAGGCGGGCATGTTTGACCAAAAGTCTTCAAGCCACGAGCGGAG
GGCATTCCTGCAGGCCATCTTGGAGCATGAGGAGGAAAATGAGGAAGAAGATGAAGTACCGGACGATGAG
ACTCTGAACCAAATGATTGCTCGACGAGAAGAAGAATTTGACCTTTTTATGCGGATGGACATGGACCGGC
GGAGGGAAGATGCCCGGAACCCGAAACGGAAGCCCCGTTTAATGGAGGAGGATGAGCTGCCCTCCTGGAT
CATTAAGGATGACGCTGAAGTAGAAAGGCTCACCTGTGAAGAAGAGGAGGAGAAAATATTTGGGAGGGGG
TCCCGCCAGCGCCGTGACGTGGACTACAGTGACGCCCTCACGGAGAAGCAGTGGCTAAGGGCCATCGAAG
ACGGCAATTTGGAGGAAATGGAAGAGGAAGTACGGCTTAAGAAGCGAAAAAGACGAAGAAATGTGGATAA
AGATCCTGCAAAAGAAGATGTGGAAAAAGCTAAGAAGAGAAGAGGCCGCCCTCCCGCTGAGAAACTGTCA
CCAAATCCCCCCAAACTGACAAAGCAGATGAACGCTATCATCGATACTGTGATAAACTACAAAGATAGGT
GTAACGTGGAGAAGGTGCCCAGTAATTCTCAGTTGGAAATAGAAGGAAACAGTTCAGGGCGACAGCTCAG
TGAAGTCTTCATTCAGTTACCTTCAAGGAAAGAATTACCAGAATACTATGAATTAATTAGGAAGCCAGTG
GATTTCAAAAAAATAAAGGAAAGGATTCGTAATCATAAGTACCGGAGCCTAGGCGACCTGGAGAAGGATG
TCATGCTTCTCTGTCACAACGCTCAGACGTTCAACCTGGAGGGATCCCAGATCTATGAAGACTCCATCGT
CTTACAGTCAGTGTTTAAGAGTGCCCGGCAGAAAATTGCCAAAGAGGAAGAGAGTGAGGATGAAAGCAAT
GAAGAGGAGGAAGAGGAAGATGAAGAAGAGTCAGAGTCCGAGGCAAAATCAGTCAAGGTGAAAATTAAGC
TCAATAAAAAAGATGACAAAGGCCGGGACAAAGGGAAAGGCAAGAAAAGGCCAAATCGAGGAAAAGCCAA
ACCTGTAGTGAGCGATTTTGACAGCGATGAGGAGCAGGATGAACGTGAACAGTCAGAAGGAAGTGGGACG
GATGATGAGTGATCAGTATGGACCTTTTTCCTTGGTAGAACTGAATTCCTTCCTCCCCTGTCTCATTTCT
ACCCAGTGAGTTCATTTGTCATATAGGCACTGGGTTGTTTCTATATCATCATCGTCTATAAACTAGCTTT
AGGATAGTGCCAGACAAACATATGATATCATGGTGTAAAAAACACACACATACACAAATATTTGTAACAT
ATTGTGACCAAATGGGCCTCAAAGATTCAGATTGAAACAAACAAAAAGCTTTTGATGGAAAATATGTGGG
TGGATAGTATATTTCTATGGGTGGGTCTAATTTGGTAACGGTTTGATTGTGCCTGGTTTTATCACCTGTT
CAGATGAGAAGATTTTTGTCTTTTGTAGCACTGATAACCAGGAGAAGCCATTAAAAGCCACTGGTTATTT
TATTTTTCATCAGGCAATTTTCGAGGTTTTTATTTGTTCGGTATTGTTTTTTTACACTGTGGTACATATA
AGCAACTTTAATAGGTGATAAATGTACAGTAGTTAGATTTCACCTGCATATACATTTTTCCATTTTATGC
TCTATGATCTGAACAAAAGCTTTTTGAATTGTATAAGATTTATGTCTACTGTAAACATTGCTTAATTTTT
TTGCTCTTGATTTAAAAAAAAGTTTTGTTGAAAGCGCTATTGAATATTGCAATCTATATAGTGTATTGGA
TGGCTTCTTTTGTCACCCTGATCTCCTATGTTACCAATGTGTATCGTCTCCTTCTCCCTAAAGTGTACTT
AATCTTTGCTTTCTTTGCACAATGTCTTTGGTTGCAAGTCATAAGCCTGAGGCAAATAAAATTCCAGTAA
TTTCGAAGAATGTGGTGTTGGTGCTTTCCTAATAAAGAAATAATTTAGCTTGACAAAAAAAAAAAAAAA
mRNA sequence of human SWI/SNF related, matrix associated, actin
dependent regulator of chromatin, subfamily a, member 2 (SMARCA2),
transcript variant 2 (GenBank Accession No. NM_139045.3)
(SEQ ID NO: 2)
GCGTCTTCCGGCGCCCGCGGAGGAGGCGAGGGTGGGACGCTGGGCGGAGCCCGAGTTTAGGAAGAGGAGG
GGACGGCTGTCATCAATGAAGTCATATTCATAATCTAGTCCTCTCTCCCTCTGTTTCTGTACTCTGGGTG
ACTCAGAGAGGGAAGAGATTCAGCCAGCACACTCCTCGCGAGCAAGCATTACTCTACTGACTGGCAGAGA
CAGGAGAGGTAGATGTCCACGCCCACAGACCCTGGTGCGATGCCCCACCCAGGGCCTTCGCCGGGGCCTG
GGCCTTCCCCTGGGCCAATTCTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGATGGGGTCCACAGACTTCCCA
CAGGAAGGCATGCATCAAATGCATAAGCCCATCGATGGTATACATGACAAGGGGATTGTAGAAGACATCC
ATTGTGGATCCATGAAGGGCACTGGTATGCGACCACCTCACCCAGGCATGGGCCCTCCCCAGAGTCCAAT
GGATCAACACAGCCAAGGTTATATGTCACCACACCCATCTCCATTAGGAGCCCCAGAGCACGTCTCCAGC
CCTATGTCTGGAGGAGGCCCAACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCTGTCCAGCTGCATCAGCTTCG
AGCTCAGATTTTAGCTTATAAAATGCTGGCCCGAGGCCAGCCCCTCCCCGAAACGCTGCAGCTTGCAGTC
CAGGGGAAAAGGACGTTGCCTGGCTTGCAGCAACAACAGCAGCAGCAACAGCAGCAGCAGCAGCAGCAGC
AGCAGCAGCAGCAGCAGCAACAGCAGCCGCAGCAGCAGCCGCCGCAACCACAGACGCAGCAACAACAGCA
GCCGGCCCTTGTTAACTACAACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGCAGCCGCGCAGCCGCCCGCGG
CCGCAGTGCCCGGGCCCTCAGTGCCGCAGCCGGCCCCGGGGCAGCCCTCGCCCGTCCTCCAGCTGCAGCA
GAAGCAGAGCCGCATCAGCCCCATCCAGAAACCGCAAGGCCTGGACCCCGTGGAAATTCTGCAAGAGCGG
GAATACAGACTTCAGGCCCGCATAGCTCATAGGATACAAGAACTGGAAAATCTGCCTGGCTCTTTGCCAC
CAGATTTAAGAACCAAAGCAACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTCTCAACTCCAAAGCATACAAA
CGGAGCAAGCGCCAGACTCTGAGAGAAGCTCGCATGACCGAGAAGCTGGAGAAGCAGCAGAAGATTGAGC
AGGAGAGGAAACGCCGTCAGAAACACCAGGAATACCTGAACAGTATTTTGCAACATGCAAAAGATTTTAA
GGAATATCATCGGTCTGTGGCCGGAAAGATCCAGAAGCTCTCCAAAGCAGTGGCAACTTGGCATGCCAAC
ACTGAAAGAGAGCAGAAGAAGGAGACAGAGCGGATTGAAAAGGAGAGAATGCGGCGACTGATGGCTGAAG
ATGAGGAGGGTTATAGAAAACTGATTGATCAAAAGAAAGACAGGCGTTTAGCTTACCTTTTGCAGCAGAC
CGATGAGTATGTAGCCAATCTGACCAATCTGGTTTGGGAGCACAAGCAAGCCCAGGCAGCCAAAGAGAAG
AAGAAGAGGAGGAGGAGGAAGAAGAAGGCTGAGGAGAATGCAGAGGGTGGGGAGTCTGCCCTGGGACCGG
ATGGAGAGCCCATAGATGAGAGCAGCCAGATGAGTGACCTCCCTGTCAAAGTGACTCACACAGAAACCGG
CAAGGTTCTGTTCGGACCAGAAGCACCCAAAGCAAGTCAGCTGGACGCCTGGCTGGAAATGAATCCTGGT
TATGAAGTTGCCCCTAGATCTGACAGTGAAGAGAGTGATTCTGATTATGAGGAAGAGGATGAGGAAGAAG
AGTCCAGTAGGCAGGAAACCGAAGAGAAAATACTCCTGGATCCAAATAGCGAAGAAGTTTCTGAGAAGGA
TGCTAAGCAGATCATTGAGACAGCTAAGCAAGACGTGGATGATGAATACAGCATGCAGTACAGTGCCAGG
GGCTCCCAGTCCTACTACACCGTGGCTCATGCCATCTCGGAGAGGGTGGAGAAACAGTCTGCCCTCCTAA
TTAATGGGACCCTAAAGCATTACCAGCTCCAGGGCCTGGAATGGATGGTTTCCCTGTATAATAACAACTT
GAACGGAATCTTAGCCGATGAAATGGGGCTTGGAAAGACCATACAGACCATTGCACTCATCACTTATCTG
ATGGAGCACAAAAGACTCAATGGCCCCTATCTCATCATTGTTCCCCTTTCGACTCTATCTAACTGGACAT
ATGAATTTGACAAATGGGCTCCTTCTGTGGTGAAGATTTCTTACAAGGGTACTCCTGCCATGCGTCGCTC
CCTTGTCCCCCAGCTACGGAGTGGCAAATTCAATGTCCTCTTGACTACTTATGAGTATATTATAAAAGAC
AAGCACATTCTTGCAAAGATTCGGTGGAAATACATGATAGTGGACGAAGGCCACCGAATGAAGAATCACC
ACTGCAAGCTGACTCAGGTCTTGAACACTCACTATGTGGCCCCCAGAAGGATCCTCTTGACTGGGACCCC
GCTGCAGAATAAGCTCCCTGAACTCTGGGCCCTCCTCAACTTCCTCCTCCCAACAATTTTTAAGAGCTGC
AGCACATTTGAACAATGGTTCAATGCTCCATTTGCCATGACTGGTGAAAGGGTGGACTTAAATGAAGAAG
AAACTATATTGATCATCAGGCGTCTACATAAGGTGTTAAGACCATTTTTACTAAGGAGACTGAAGAAAGA
AGTTGAATCCCAGCTTCCCGAAAAAGTGGAATATGTGATCAAGTGTGACATGTCAGCTCTGCAGAAGATT
CTGTATCGCCATATGCAAGCCAAGGGGATCCTTCTCACAGATGGTTCTGAGAAAGATAAGAAGGGGAAAG
GAGGTGCTAAGACACTTATGAACACTATTATGCAGTTGAGAAAAATCTGCAACCACCCATATATGTTTCA
GCACATTGAGGAATCCTTTGCTGAACACCTAGGCTATTCAAATGGGGTCATCAATGGGGCTGAACTGTAT
CGGGCCTCAGGGAAGTTTGAGCTGCTTGATCGTATTCTGCCAAAATTGAGAGCGACTAATCACCGAGTGC
TGCTTTTCTGCCAGATGACATCTCTCATGACCATCATGGAGGATTATTTTGCTTTTCGGAACTTCCTTTA
CCTACGCCTTGATGGCACCACCAAGTCTGAAGATCGTGCTGCTTTGCTGAAGAAATTCAATGAACCTGGA
TCCCAGTATTTCATTTTCTTGCTGAGCACAAGAGCTGGTGGCCTGGGCTTAAATCTTCAGGCAGCTGATA
CAGTGGTCATCTTTGACAGCGACTGGAATCCTCATCAGGATCTGCAGGCCCAAGACCGAGCTCACCGCAT
CGGGCAGCAGAACGAGGTCCGGGTACTGAGGCTCTGTACCGTGAACAGCGTGGAGGAAAAGATCCTCGCG
GCCGCAAAATACAAGCTGAACGTGGATCAGAAAGTGATCCAGGCGGGCATGTTTGACCAAAAGTCTTCAA
GCCACGAGCGGAGGGCATTCCTGCAGGCCATCTTGGAGCATGAGGAGGAAAATGAGGAAGAAGATGAAGT
ACCGGACGATGAGACTCTGAACCAAATGATTGCTCGACGAGAAGAAGAATTTGACCTTTTTATGCGGATG
GACATGGACCGGCGGAGGGAAGATGCCCGGAACCCGAAACGGAAGCCCCGTTTAATGGAGGAGGATGAGC
TGCCCTCCTGGATCATTAAGGATGACGCTGAAGTAGAAAGGCTCACCTGTGAAGAAGAGGAGGAGAAAAT
ATTTGGGAGGGGGTCCCGCCAGCGCCGTGACGTGGACTACAGTGACGCCCTCACGGAGAAGCAGTGGCTA
AGGGCCATCGAAGACGGCAATTTGGAGGAAATGGAAGAGGAAGTACGGCTTAAGAAGCGAAAAAGACGAA
GAAATGTGGATAAAGATCCTGCAAAAGAAGATGTGGAAAAAGCTAAGAAGAGAAGAGGCCGCCCTCCCGC
TGAGAAACTGTCACCAAATCCCCCCAAACTGACAAAGCAGATGAACGCTATCATCGATACTGTGATAAAC
TACAAAGATAGTTCAGGGCGACAGCTCAGTGAAGTCTTCATTCAGTTACCTTCAAGGAAAGAATTACCAG
AATACTATGAATTAATTAGGAAGCCAGTGGATTTCAAAAAAATAAAGGAAAGGATTCGTAATCATAAGTA
CCGGAGCCTAGGCGACCTGGAGAAGGATGTCATGCTTCTCTGTCACAACGCTCAGACGTTCAACCTGGAG
GGATCCCAGATCTATGAAGACTCCATCGTCTTACAGTCAGTGTTTAAGAGTGCCCGGCAGAAAATTGCCA
AAGAGGAAGAGAGTGAGGATGAAAGCAATGAAGAGGAGGAAGAGGAAGATGAAGAAGAGTCAGAGTCCGA
GGCAAAATCAGTCAAGGTGAAAATTAAGCTCAATAAAAAAGATGACAAAGGCCGGGACAAAGGGAAAGGC
AAGAAAAGGCCAAATCGAGGAAAAGCCAAACCTGTAGTGAGCGATTTTGACAGCGATGAGGAGCAGGATG
AACGTGAACAGTCAGAAGGAAGTGGGACGGATGATGAGTGATCAGTATGGACCTTTTTCCTTGGTAGAAC
TGAATTCCTTCCTCCCCTGTCTCATTTCTACCCAGTGAGTTCATTTGTCATATAGGCACTGGGTTGTTTC
TATATCATCATCGTCTATAAACTAGCTTTAGGATAGTGCCAGACAAACATATGATATCATGGTGTAAAAA
ACACACACATACACAAATATTTGTAACATATTGTGACCAAATGGGCCTCAAAGATTCAGATTGAAACAAA
CAAAAAGCTTTTGATGGAAAATATGTGGGTGGATAGTATATTTCTATGGGTGGGTCTAATTTGGTAACGG
TTTGATTGTGCCTGGTTTTATCACCTGTTCAGATGAGAAGATTTTTGTCTTTTGTAGCACTGATAACCAG
GAGAAGCCATTAAAAGCCACTGGTTATTTTATTTTTCATCAGGCAATTTTCGAGGTTTTTATTTGTTCGG
TATTGTTTTTTTACACTGTGGTACATATAAGCAACTTTAATAGGTGATAAATGTACAGTAGTTAGATTTC
ACCTGCATATACATTTTTCCATTTTATGCTCTATGATCTGAACAAAAGCTTTTTGAATTGTATAAGATTT
ATGTCTACTGTAAACATTGCTTAATTTTTTTGCTCTTGATTTAAAAAAAAGTTTTGTTGAAAGCGCTATT
GAATATTGCAATCTATATAGTGTATTGGATGGCTTCTTTTGTCACCCTGATCTCCTATGTTACCAATGTG
TATCGTCTCCTTCTCCCTAAAGTGTACTTAATCTTTGCTTTCTTTGCACAATGTCTTTGGTTGCAAGTCA
TAAGCCTGAGGCAAATAAAATTCCAGTAATTTCGAAGAATGTGGTGTTGGTGCTTTCCTAATAAAGAAAT
AATTTAGCTTGACAAAAAAAAAAAAAAA
mRNA sequence of human SWI/SNF related, matrix associated, actin
dependent regulator of chromatin, subfamily a, member 2 (SMARCA2),
transcript variant 4 (GenBank Accession No. NM_001289397.1)
(SEQ ID NO: 3)
GCGTCTTCCGGCGCCCGCGGAGGAGGCGAGGGTGGGACGCTGGGCGGAGCCCGAGTTTAGGAAGAGGAGG
GGACGGCTGTCATCAATGAAGTCATATTCATAATCTAGTCCTCTCTCCCTCTGTTTCTGTACTCTGGGTG
ACTCAGAGAGGGAAGAGATTCAGCCAGCACACTCCTCGCGAGCAAGCATTACTCTACTGACTGGCAGAGA
CAGGAGAGGTAGATGTCCACGCCCACAGACCCTGGTGCGATGCCCCACCCAGGGCCTTCGCCGGGGCCTG
GGCCTTCCCCTGGGCCAATTCTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGATGGGGTCCACAGACTTCCCA
CAGGAAGGCATGCATCAAATGCATAAGCCCATCGATGGTATACATGACAAGGGGATTGTAGAAGACATCC
ATTGTGGATCCATGAAGGGCACTGGTATGCGACCACCTCACCCAGGCATGGGCCCTCCCCAGAGTCCAAT
GGATCAACACAGCCAAGGTTATATGTCACCACACCCATCTCCATTAGGAGCCCCAGAGCACGTCTCCAGC
CCTATGTCTGGAGGAGGCCCAACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCTGTCCAGCTGCATCAGCTTCG
AGCTCAGATTTTAGCTTATAAAATGCTGGCCCGAGGCCAGCCCCTCCCCGAAACGCTGCAGCTTGCAGTC
CAGGGGAAAAGGACGTTGCCTGGCTTGCAGCAACAACAGCAGCAGCAACAGCAGCAGCAGCAGCAGCAGC
AGCAGCAGCAGCAGCAGCAACAGCAGCCGCAGCAGCAGCCGCCGCAACCACAGACGCAGCAACAACAGCA
GCCGGCCCTTGTTAACTACAACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGCAGCCGCGCAGCCGCCCGCGG
CCGCAGTGCCCGGGCCCTCAGTGCCGCAGCCGGCCCCGGGGCAGCCCTCGCCCGTCCTCCAGCTGCAGCA
GAAGCAGAGCCGCATCAGCCCCATCCAGAAACCGCAAGGCCTGGACCCCGTGGAAATTCTGCAAGAGCGG
GAATACAGACTTCAGGCCCGCATAGCTCATAGGATACAAGAACTGGAAAATCTGCCTGGCTCTTTGCCAC
CAGATTTAAGAACCAAAGCAACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTCTCAACTCCAAAGCATACAAA
CGGAGCAAGCGCCAGACTCTGAGAGAAGCTCGCATGACCGAGAAGCTGGAGAAGCAGCAGAAGATTGAGC
AGGAGAGGAAACGCCGTCAGAAACACCAGGAATACCTGAACAGTATTTTGCAACATGCAAAAGATTTTAA
GGAATATCATCGGTCTGTGGCCGGAAAGATCCAGAAGCTCTCCAAAGCAGTGGCAACTTGGCATGCCAAC
ACTGAAAGAGAGCAGAAGAAGGAGACAGAGCGGATTGAAAAGGAGAGAATGCGGCGACTGATGGCTGAAG
ATGAGGAGGGTTATAGAAAACTGATTGATCAAAAGAAAGACAGGCGTTTAGCTTACCTTTTGCAGCAGAC
CGATGAGTATGTAGCCAATCTGACCAATCTGGTTTGGGAGCACAAGCAAGCCCAGGCAGCCAAAGAGAAG
AAGAAGAGGAGGAGGAGGAAGAAGAAGGCTGAGGAGAATGCAGAGGGTGGGGAGTCTGCCCTGGGACCGG
ATGGAGAGCCCATAGATGAGAGCAGCCAGATGAGTGACCTCCCTGTCAAAGTGACTCACACAGAAACCGG
CAAGGTTCTGTTCGGACCAGAAGCACCCAAAGCAAGTCAGCTGGACGCCTGGCTGGAAATGAATCCTGGT
TATGAAGTTGCCCCTAGATCTGACAGTGAAGAGAGTGATTCTGATTATGAGGAAGAGGATGAGGAAGAAG
AGTCCAGTAGGCAGGAAACCGAAGAGAAAATACTCCTGGATCCAAATAGCGAAGAAGTTTCTGAGAAGGA
TGCTAAGCAGATCATTGAGACAGCTAAGCAAGACGTGGATGATGAATACAGCATGCAGTACAGTGCCAGG
GGCTCCCAGTCCTACTACACCGTGGCTCATGCCATCTCGGAGAGGGTGGAGAAACAGTCTGCCCTCCTAA
TTAATGGGACCCTAAAGCATTACCAGCTCCAGGGCCTGGAATGGATGGTTTCCCTGTATAATAACAACTT
GAACGGAATCTTAGCCGATGAAATGGGGCTTGGAAAGACCATACAGACCATTGCACTCATCACTTATCTG
ATGGAGCACAAAAGACTCAATGGCCCCTATCTCATCATTGTTCCCCTTTCGACTCTATCTAACTGGACAT
ATGAATTTGACAAATGGGCTCCTTCTGTGGTGAAGATTTCTTACAAGGGTACTCCTGCCATGCGTCGCTC
CCTTGTCCCCCAGCTACGGAGTGGCAAATTCAATGTCCTCTTGACTACTTATGAGTATATTATAAAAGAC
AAGCACATTCTTGCAAAGATTCGGTGGAAATACATGATAGTGGACGAAGGCCACCGAATGAAGAATCACC
ACTGCAAGCTGACTCAGGTGGACTTAAATGAAGAAGAAACTATATTGATCATCAGGCGTCTACATAAGGT
GTTAAGACCATTTTTACTAAGGAGACTGAAGAAAGAAGTTGAATCCCAGCTTCCCGAAAAAGTGGAATAT
GTGATCAAGTGTGACATGTCAGCTCTGCAGAAGATTCTGTATCGCCATATGCAAGCCAAGGGGATCCTTC
TCACAGATGGTTCTGAGAAAGATAAGAAGGGGAAAGGAGGTGCTAAGACACTTATGAACACTATTATGCA
GTTGAGAAAAATCTGCAACCACCCATATATGTTTCAGCACATTGAGGAATCCTTTGCTGAACACCTAGGC
TATTCAAATGGGGTCATCAATGGGGCTGAACTGTATCGGGCCTCAGGGAAGTTTGAGCTGCTTGATCGTA
TTCTGCCAAAATTGAGAGCGACTAATCACCGAGTGCTGCTTTTCTGCCAGATGACATCTCTCATGACCAT
CATGGAGGATTATTTTGCTTTTCGGAACTTCCTTTACCTACGCCTTGATGGCACCACCAAGTCTGAAGAT
CGTGCTGCTTTGCTGAAGAAATTCAATGAACCTGGATCCCAGTATTTCATTTTCTTGCTGAGCACAAGAG
CTGGTGGCCTGGGCTTAAATCTTCAGGCAGCTGATACAGTGGTCATCTTTGACAGCGACTGGAATCCTCA
TCAGGATCTGCAGGCCCAAGACCGAGCTCACCGCATCGGGCAGCAGAACGAGGTCCGGGTACTGAGGCTC
TGTACCGTGAACAGCGTGGAGGAAAAGATCCTCGCGGCCGCAAAATACAAGCTGAACGTGGATCAGAAAG
TGATCCAGGCGGGCATGTTTGACCAAAAGTCTTCAAGCCACGAGCGGAGGGCATTCCTGCAGGCCATCTT
GGAGCATGAGGAGGAAAATGAGGAAGAAGATGAAGTACCGGACGATGAGACTCTGAACCAAATGATTGCT
CGACGAGAAGAAGAATTTGACCTTTTTATGCGGATGGACATGGACCGGCGGAGGGAAGATGCCCGGAACC
CGAAACGGAAGCCCCGTTTAATGGAGGAGGATGAGCTGCCCTCCTGGATCATTAAGGATGACGCTGAAGT
AGAAAGGCTCACCTGTGAAGAAGAGGAGGAGAAAATATTTGGGAGGGGGTCCCGCCAGCGCCGTGACGTG
GACTACAGTGACGCCCTCACGGAGAAGCAGTGGCTAAGGGCCATCGAAGACGGCAATTTGGAGGAAATGG
AAGAGGAAGTACGGCTTAAGAAGCGAAAAAGACGAAGAAATGTGGATAAAGATCCTGCAAAAGAAGATGT
GGAAAAAGCTAAGAAGAGAAGAGGCCGCCCTCCCGCTGAGAAACTGTCACCAAATCCCCCCAAACTGACA
AAGCAGATGAACGCTATCATCGATACTGTGATAAACTACAAAGATAGTTCAGGGCGACAGCTCAGTGAAG
TCTTCATTCAGTTACCTTCAAGGAAAGAATTACCAGAATACTATGAATTAATTAGGAAGCCAGTGGATTT
CAAAAAAATAAAGGAAAGGATTCGTAATCATAAGTACCGGAGCCTAGGCGACCTGGAGAAGGATGTCATG
CTTCTCTGTCACAACGCTCAGACGTTCAACCTGGAGGGATCCCAGATCTATGAAGACTCCATCGTCTTAC
AGTCAGTGTTTAAGAGTGCCCGGCAGAAAATTGCCAAAGAGGAAGAGAGTGAGGATGAAAGCAATGAAGA
GGAGGAAGAGGAAGATGAAGAAGAGTCAGAGTCCGAGGCAAAATCAGTCAAGGTGAAAATTAAGCTCAAT
AAAAAAGATGACAAAGGCCGGGACAAAGGGAAAGGCAAGAAAAGGCCAAATCGAGGAAAAGCCAAACCTG
TAGTGAGCGATTTTGACAGCGATGAGGAGCAGGATGAACGTGAACAGTCAGAAGGAAGTGGGACGGATGA
TGAGTGATCAGTATGGACCTTTTTCCTTGGTAGAACTGAATTCCTTCCTCCCCTGTCTCATTTCTACCCA
GTGAGTTCATTTGTCATATAGGCACTGGGTTGTTTCTATATCATCATCGTCTATAAACTAGCTTTAGGAT
AGTGCCAGACAAACATATGATATCATGGTGTAAAAAACACACACATACACAAATATTTGTAACATATTGT
GACCAAATGGGCCTCAAAGATTCAGATTGAAACAAACAAAAAGCTTTTGATGGAAAATATGTGGGTGGAT
AGTATATTTCTATGGGTGGGTCTAATTTGGTAACGGTTTGATTGTGCCTGGTTTTATCACCTGTTCAGAT
GAGAAGATTTTTGTCTTTTGTAGCACTGATAACCAGGAGAAGCCATTAAAAGCCACTGGTTATTTTATTT
TTCATCAGGCAATTTTCGAGGTTTTTATTTGTTCGGTATTGTTTTTTTACACTGTGGTACATATAAGCAA
CTTTAATAGGTGATAAATGTACAGTAGTTAGATTTCACCTGCATATACATTTTTCCATTTTATGCTCTAT
GATCTGAACAAAAGCTTTTTGAATTGTATAAGATTTATGTCTACTGTAAACATTGCTTAATTTTTTTGCT
CTTGATTTAAAAAAAAGTTTTGTTGAAAGCGCTATTGAATATTGCAATCTATATAGTGTATTGGATGGCT
TCTTTTGTCACCCTGATCTCCTATGTTACCAATGTGTATCGTCTCCTTCTCCCTAAAGTGTACTTAATCT
TTGCTTTCTTTGCACAATGTCTTTGGTTGCAAGTCATAAGCCTGAGGCAAATAAAATTCCAGTAATTTCG
AAGAATGTGGTGTTGGTGCTTTCCTAATAAAGAAATAATTTAGCTTGACAAAAAAAAAAAAAAA
mRNA sequence of human SWI/SNF related, matrix associated, actin
dependent regulator of chromatin, subfamily a, member 2 (SMARCA2),
transcript variant 5 (GenBank Accession No. NM_001289398.1)
(SEQ ID NO: 4)
CTTGGAGAGGCGGAGGTGGAAACGATGCGCAGGAGTTGGCTTGGGGCTTTTTGTTTGCGTGTCCCTGTTT
ACCTATTCATAATCATGGATCCCCTCTGCTTTGTGATACTGTGAACCACGCATAACAGCAATTCTTTACA
CCACCGGGTTGAGAAGAAGGCGCCTGAGGCTGACTTTCTGGACCTGCCGTCACGCAGTAAAGATGTGGTT
GGCCATCGAAGACGGCAATTTGGAGGAAATGGAAGAGGAAGTACGGCTTAAGAAGCGAAAAAGACGAAGA
AATGTGGATAAAGATCCTGCAAAAGAAGATGTGGAAAAAGCTAAGAAGAGAAGAGGCCGCCCTCCCGCTG
AGAAACTGTCACCAAATCCCCCCAAACTGACAAAGCAGATGAACGCTATCATCGATACTGTGATAAACTA
CAAAGATAGTTCAGGGCGACAGCTCAGTGAAGTCTTCATTCAGTTACCTTCAAGGAAAGAATTACCAGAA
TACTATGAATTAATTAGGAAGCCAGTGGATTTCAAAAAAATAAAGGAAAGGATTCGTAATCATAAGTACC
GGAGCCTAGGCGACCTGGAGAAGGATGTCATGCTTCTCTGTCACAACGCTCAGACGTTCAACCTGGAGGG
ATCCCAGATCTATGAAGACTCCATCGTCTTACAGTCAGTGTTTAAGAGTGCCCGGCAGAAAATTGCCAAA
GAGGAAGAGAGTGAGGATGAAAGCAATGAAGAGGAGGAAGAGGAAGATGAAGAAGAGTCAGAGTCCGAGG
CAAAATCAGTCAAGGTGAAAATTAAGCTCAATAAAAAAGATGACAAAGGCCGGGACAAAGGGAAAGGCAA
GAAAAGGCCAAATCGAGGAAAAGCCAAACCTGTAGTGAGCGATTTTGACAGCGATGAGGAGCAGGATGAA
CGTGAACAGTCAGAAGGAAGTGGGACGGATGATGAGTGATCAGTATGGACCTTTTTCCTTGGTAGAACTG
AATTCCTTCCTCCCCTGTCTCATTTCTACCCAGTGAGTTCATTTGTCATATAGGCACTGGGTTGTTTCTA
TATCATCATCGTCTATAAACTAGCTTTAGGATAGTGCCAGACAAACATATGATATCATGGTGTAAAAAAC
ACACACATACACAAATATTTGTAACATATTGTGACCAAATGGGCCTCAAAGATTCAGATTGAAACAAACA
AAAAGCTTTTGATGGAAAATATGTGGGTGGATAGTATATTTCTATGGGTGGGTCTAATTTGGTAACGGTT
TGATTGTGCCTGGTTTTATCACCTGTTCAGATGAGAAGATTTTTGTCTTTTGTAGCACTGATAACCAGGA
GAAGCCATTAAAAGCCACTGGTTATTTTATTTTTCATCAGGCAATTTTCGAGGTTTTTATTTGTTCGGTA
TTGTTTTTTTACACTGTGGTACATATAAGCAACTTTAATAGGTGATAAATGTACAGTAGTTAGATTTCAC
CTGCATATACATTTTTCCATTTTATGCTCTATGATCTGAACAAAAGCTTTTTGAATTGTATAAGATTTAT
GTCTACTGTAAACATTGCTTAATTTTTTTGCTCTTGATTTAAAAAAAAGTTTTGTTGAAAGCGCTATTGA
ATATTGCAATCTATATAGTGTATTGGATGGCTTCTTTTGTCACCCTGATCTCCTATGTTACCAATGTGTA
TCGTCTCCTTCTCCCTAAAGTGTACTTAATCTTTGCTTTCTTTGCACAATGTCTTTGGTTGCAAGTCATA
AGCCTGAGGCAAATAAAATTCCAGTAATTTCGAAGAATGTGGTGTTGGTGCTTTCCTAATAAAGAAATAA
TTTAGCTTGACAAAAAAAAAAAAAAA
Protein sequence of human probable global transcription activator
SNF2L2 isoform a (GenBank Accession No. NP_001276325.1)
(SEQ ID NO: 5)
MSTPTDPGAMPHPGPSPGPGPSPGPILGPSPGPGPSPGSVHSMMGPSPGPPSVSHPMPTMGSTDFPQEGM
HQMHKPIDGIHDKGIVEDIHCGSMKGTGMRPPHPGMGPPQSPMDQHSQGYMSPHPSPLGAPEHVSSPMSG
GGPTPPQMPPSQPGALIPGDPQAMSQPNRGPSPFSPVQLHQLRAQILAYKMLARGQPLPETLQLAVQGKR
TLPGLQQQQQQQQQQQQQQQQQQQQQQQPQQQPPQPQTQQQQQPALVNYNRPSGPGPELSGPSTPQKLPV
PAPGGRPSPAPPAAAQPPAAAVPGPSVPQPAPGQPSPVLQLQQKQSRISPIQKPQGLDPVEILQEREYRL
QARIAHRIQELENLPGSLPPDLRTKATVELKALRLLNFQRQLRQEVVACMRRDTTLETALNSKAYKRSKR
QTLREARMTEKLEKQQKIEQERKRRQKHQEYLNSILQHAKDFKEYHRSVAGKIQKLSKAVATWHANTERE
QKKETERIEKERMRRLMAEDEEGYRKLIDQKKDRRLAYLLQQTDEYVANLTNLVWEHKQAQAAKEKKKRR
RRKKKAEENAEGGESALGPDGEPIDESSQMSDLPVKVTHTETGKVLFGPEAPKASQLDAWLEMNPGYEVA
PRSDSEESDSDYEEEDEEEESSRQETEEKILLDPNSEEVSEKDAKQIIETAKQDVDDEYSMQYSARGSQS
YYTVAHAISERVEKQSALLINGTLKHYQLQGLEWMVSLYNNNLNGILADEMGLGKTIQTIALITYLMEHK
RLNGPYLIIVPLSTLSNWTYEFDKWAPSVVKISYKGTPAMRRSLVPQLRSGKFNVLLTTYEYIIKDKHIL
AKIRWKYMIVDEGHRMKNHHCKLTQVLNTHYVAPRRILLTGTPLQNKLPELWALLNFLLPTIFKSCSTFE
QWFNAPFAMTGERVDLNEEETILIIRRLHKVLRPFLLRRLKKEVESQLPEKVEYVIKCDMSALQKILYRH
MQAKGILLTDGSEKDKKGKGGAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTTKSEDRAALLKKFNEPGSQYF
IFLLSTRAGGLGLNLQAADTVVIFDSDWNPHQDLQAQDRAHRIGQQNEVRVLRLCTVNSVEEKILAAAKY
KLNVDQKVIQAGMFDQKSSSHERRAFLQAILEHEEENEEEDEVPDDETLNQMIARREEEFDLFMRMDMDR
RREDARNPKRKPRLMEEDELPSWIIKDDAEVERLTCEEEEEKIFGRGSRQRRDVDYSDALTEKQWLRAIE
DGNLEEMEEEVRLKKRKRRRNVDKDPAKEDVEKAKKRRGRPPAEKLSPNPPKLTKQMNAIIDTVINYKDR
CNVEKVPSNSQLEIEGNSSGRQLSEVFIQLPSRKELPEYYELIRKPVDFKKIKERIRNHKYRSLGDLEKD
VMLLCHNAQTFNLEGSQIYEDSIVLQSVFKSARQKIAKEEESEDESNEEEEEEDEEESESEAKSVKVKIK
LNKKDDKGRDKGKGKKRPNRGKAKPVVSDFDSDEEQDEREQSEGSGTDDE
Protein sequence of human probable global transcription activator
SNF2L2 isoform b (GenBank Accession No. NP_620614.2)
(SEQ ID NO: 6)
MSTPTDPGAMPHPGPSPGPGPSPGPILGPSPGPGPSPGSVHSMMGPSPGPPSVSHPMPTMGSTDFPQEGM
HQMHKPIDGIHDKGIVEDIHCGSMKGTGMRPPHPGMGPPQSPMDQHSQGYMSPHPSPLGAPEHVSSPMSG
GGPTPPQMPPSQPGALIPGDPQAMSQPNRGPSPFSPVQLHQLRAQILAYKMLARGQPLPETLQLAVQGKR
TLPGLQQQQQQQQQQQQQQQQQQQQQQQPQQQPPQPQTQQQQQPALVNYNRPSGPGPELSGPSTPQKLPV
PAPGGRPSPAPPAAAQPPAAAVPGPSVPQPAPGQPSPVLQLQQKQSRISPIQKPQGLDPVEILQEREYRL
QARIAHRIQELENLPGSLPPDLRTKATVELKALRLLNFQRQLRQEVVACMRRDTTLETALNSKAYKRSKR
QTLREARMTEKLEKQQKIEQERKRRQKHQEYLNSILQHAKDFKEYHRSVAGKIQKLSKAVATWHANTERE
QKKETERIEKERMRRLMAEDEEGYRKLIDQKKDRRLAYLLQQTDEYVANLTNLVWEHKQAQAAKEKKKRR
RRKKKAEENAEGGESALGPDGEPIDESSQMSDLPVKVTHTETGKVLFGPEAPKASQLDAWLEMNPGYEVA
PRSDSEESDSDYEEEDEEEESSRQETEEKILLDPNSEEVSEKDAKQIIETAKQDVDDEYSMQYSARGSQS
YYTVAHAISERVEKQSALLINGTLKHYQLQGLEWMVSLYNNNLNGILADEMGLGKTIQTIALITYLMEHK
RLNGPYLIIVPLSTLSNWTYEFDKWAPSVVKISYKGTPAMRRSLVPQLRSGKFNVLLTTYEYIIKDKHIL
AKIRWKYMIVDEGHRMKNHHCKLTQVLNTHYVAPRRILLTGTPLQNKLPELWALLNFLLPTIFKSCSTFE
QWFNAPFAMTGERVDLNEEETILIIRRLHKVLRPFLLRRLKKEVESQLPEKVEYVIKCDMSALQKILYRH
MQAKGILLTDGSEKDKKGKGGAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTTKSEDRAALLKKFNEPGSQYF
IFLLSTRAGGLGLNLQAADTVVIFDSDWNPHQDLQAQDRAHRIGQQNEVRVLRLCTVNSVEEKILAAAKY
KLNVDQKVIQAGMFDQKSSSHERRAFLQAILEHEEENEEEDEVPDDETLNQMIARREEEFDLFMRMDMDR
RREDARNPKRKPRLMEEDELPSWIIKDDAEVERLTCEEEEEKIFGRGSRQRRDVDYSDALTEKQWLRAIE
DGNLEEMEEEVRLKKRKRRRNVDKDPAKEDVEKAKKRRGRPPAEKLSPNPPKLTKQMNAIIDTVINYKDS
SGRQLSEVFIQLPSRKELPEYYELIRKPVDFKKIKERIRNHKYRSLGDLEKDVMLLCHNAQTFNLEGSQI
YEDSIVLQSVFKSARQKIAKEEESEDESNEEEEEEDEEESESEAKSVKVKIKLNKKDDKGRDKGKGKKRP
NRGKAKPVVSDFDSDEEQDEREQSEGSGTDDE
Protein sequence of human probable global transcription activator
SNF2L2 isoform c (GenBank Accession No. NP_001276326.1)
(SEQ ID NO: 7)
MSTPTDPGAMPHPGPSPGPGPSPGPILGPSPGPGPSPGSVHSMMGPSPGPPSVSHPMPTMGSTDFPQEGM
HQMHKPIDGIHDKGIVEDIHCGSMKGTGMRPPHPGMGPPQSPMDQHSQGYMSPHPSPLGAPEHVSSPMSG
GGPTPPQMPPSQPGALIPGDPQAMSQPNRGPSPFSPVQLHQLRAQILAYKMLARGQPLPETLQLAVQGKR
TLPGLQQQQQQQQQQQQQQQQQQQQQQQPQQQPPQPQTQQQQQPALVNYNRPSGPGPELSGPSTPQKLPV
PAPGGRPSPAPPAAAQPPAAAVPGPSVPQPAPGQPSPVLQLQQKQSRISPIQKPQGLDPVEILQEREYRL
QARIAHRIQELENLPGSLPPDLRTKATVELKALRLLNFQRQLRQEVVACMRRDTTLETALNSKAYKRSKR
QTLREARMTEKLEKQQKIEQERKRRQKHQEYLNSILQHAKDFKEYHRSVAGKIQKLSKAVATWHANTERE
QKKETERIEKERMRRLMAEDEEGYRKLIDQKKDRRLAYLLQQTDEYVANLTNLVWEHKQAQAAKEKKKRR
RRKKKAEENAEGGESALGPDGEPIDESSQMSDLPVKVTHTETGKVLFGPEAPKASQLDAWLEMNPGYEVA
PRSDSEESDSDYEEEDEEEESSRQETEEKILLDPNSEEVSEKDAKQIIETAKQDVDDEYSMQYSARGSQS
YYTVAHAISERVEKQSALLINGTLKHYQLQGLEWMVSLYNNNLNGILADEMGLGKTIQTIALITYLMEHK
RLNGPYLIIVPLSTLSNWTYEFDKWAPSVVKISYKGTPAMRRSLVPQLRSGKFNVLLTTYEYIIKDKHIL
AKIRWKYMIVDEGHRMKNHHCKLTQVDLNEEETILIIRRLHKVLRPFLLRRLKKEVESQLPEKVEYVIKC
DMSALQKILYRHMQAKGILLTDGSEKDKKGKGGAKTLMNTIMQLRKICNHPYMFQHIEESFAEHLGYSNG
VINGAELYRASGKFELLDRILPKLRATNHRVLLFCQMTSLMTIMEDYFAFRNFLYLRLDGTTKSEDRAAL
LKKFNEPGSQYFIFLLSTRAGGLGLNLQAADTVVIFDSDWNPHQDLQAQDRAHRIGQQNEVRVLRLCTVN
SVEEKILAAAKYKLNVDQKVIQAGMFDQKSSSHERRAFLQAILEHEEENEEEDEVPDDETLNQMIARREE
EFDLFMRMDMDRRREDARNPKRKPRLMEEDELPSWIIKDDAEVERLTCEEEEEKIFGRGSRQRRDVDYSD
ALTEKQWLRAIEDGNLEEMEEEVRLKKRKRRRNVDKDPAKEDVEKAKKRRGRPPAEKLSPNPPKLTKQMN
AIIDTVINYKDSSGRQLSEVFIQLPSRKELPEYYELIRKPVDFKKIKERIRNHKYRSLGDLEKDVMLLCH
NAQTFNLEGSQIYEDSIVLQSVFKSARQKIAKEEESEDESNEEEEEEDEEESESEAKSVKVKIKLNKKDD
KGRDKGKGKKRPNRGKAKPVVSDFDSDEEQDEREQSEGSGTDDE
Protein sequence of human probable global transcription activator
SNF2L2 isoform d (GenBank Accession No. NP_001276327.1)
(SEQ ID NO: 8)
MWLAIEDGNLEEMEEEVRLKKRKRRRNVDKDPAKEDVEKAKKRRGRPPAEKLSPNPPKLTKQMNAIIDTV
INYKDSSGRQLSEVFIQLPSRKELPEYYELIRKPVDFKKIKERIRNHKYRSLGDLEKDVMLLCHNAQTFN
LEGSQIYEDSIVLQSVFKSARQKIAKEEESEDESNEEEEEEDEEESESEAKSVKVKIKLNKKDDKGRDKG
KGKKRPNRGKAKPVVSDFDSDEEQDEREQSEGSGTDDE
SMARCA4
mRNA sequence of human SWI/SNF related, matrix associated, actin
dependent regulator of chromatin, subfamily a, member 4 (SMARCA4),
transcript variant 1 (GenBank Accession No. NM_001128849.1)
(SEQ ID NO: 9)
GGCGGGGGAGGCGCCGGGAAGTCGACGGCGCCGGCGGCTCCTGCAGGAGGCCACTGTCTGCAGCTCCCGT
GAAGATGTCCACTCCAGACCCACCCCTGGGCGGAACTCCTCGGCCAGGTCCTTCCCCGGGCCCTGGCCCT
TCCCCTGGAGCCATGCTGGGCCCTAGCCCGGGTCCCTCGCCGGGCTCCGCCCACAGCATGATGGGGCCCA
GCCCAGGGCCGCCCTCAGCAGGACACCCCATCCCCACCCAGGGGCCTGGAGGGTACCCTCAGGACAACAT
GCACCAGATGCACAAGCCCATGGAGTCCATGCATGAGAAGGGCATGTCGGACGACCCGCGCTACAACCAG
ATGAAAGGAATGGGGATGCGGTCAGGGGGCCATGCTGGGATGGGGCCCCCGCCCAGCCCCATGGACCAGC
ACTCCCAAGGTTACCCCTCGCCCCTGGGTGGCTCTGAGCATGCCTCTAGTCCAGTTCCAGCCAGTGGCCC
GTCTTCGGGGCCCCAGATGTCTTCCGGGCCAGGAGGTGCCCCGCTGGATGGTGCTGACCCCCAGGCCTTG
GGGCAGCAGAACCGGGGCCCAACCCCATTTAACCAGAACCAGCTGCACCAGCTCAGAGCTCAGATCATGG
CCTACAAGATGCTGGCCAGGGGGCAGCCCCTCCCCGACCACCTGCAGATGGCGGTGCAGGGCAAGCGGCC
GATGCCCGGGATGCAGCAGCAGATGCCAACGCTACCTCCACCCTCGGTGTCCGCAACAGGACCCGGCCCT
GGCCCTGGCCCTGGCCCCGGCCCGGGTCCCGGCCCGGCACCTCCAAATTACAGCAGGCCTCATGGTATGG
GAGGGCCCAACATGCCTCCCCCAGGACCCTCGGGCGTGCCCCCCGGGATGCCAGGCCAGCCTCCTGGAGG
GCCTCCCAAGCCCTGGCCTGAAGGACCCATGGCGAATGCTGCTGCCCCCACGAGCACCCCTCAGAAGCTG
ATTCCCCCGCAGCCAACGGGCCGCCCTTCCCCCGCGCCCCCTGCCGTCCCACCCGCCGCCTCGCCCGTGA
TGCCACCGCAGACCCAGTCCCCCGGGCAGCCGGCCCAGCCCGCGCCCATGGTGCCACTGCACCAGAAGCA
GAGCCGCATCACCCCCATCCAGAAGCCGCGGGGCCTCGACCCTGTGGAGATCCTGCAGGAGCGCGAGTAC
AGGCTGCAGGCTCGCATCGCACACCGAATTCAGGAACTTGAAAACCTTCCCGGGTCCCTGGCCGGGGATT
TGCGAACCAAAGCGACCATTGAGCTCAAGGCCCTCAGGCTGCTGAACTTCCAGAGGCAGCTGCGCCAGGA
GGTGGTGGTGTGCATGCGGAGGGACACAGCGCTGGAGACAGCCCTCAATGCTAAGGCCTACAAGCGCAGC
AAGCGCCAGTCCCTGCGCGAGGCCCGCATCACTGAGAAGCTGGAGAAGCAGCAGAAGATCGAGCAGGAGC
GCAAGCGCCGGCAGAAGCACCAGGAATACCTCAATAGCATTCTCCAGCATGCCAAGGATTTCAAGGAATA
TCACAGATCCGTCACAGGCAAAATCCAGAAGCTGACCAAGGCAGTGGCCACGTACCATGCCAACACGGAG
CGGGAGCAGAAGAAAGAGAACGAGCGGATCGAGAAGGAGCGCATGCGGAGGCTCATGGCTGAAGATGAGG
AGGGGTACCGCAAGCTCATCGACCAGAAGAAGGACAAGCGCCTGGCCTACCTCTTGCAGCAGACAGACGA
GTACGTGGCTAACCTCACGGAGCTGGTGCGGCAGCACAAGGCTGCCCAGGTCGCCAAGGAGAAAAAGAAG
AAAAAGAAAAAGAAGAAGGCAGAAAATGCAGAAGGACAGACGCCTGCCATTGGGCCGGATGGCGAGCCTC
TGGACGAGACCAGCCAGATGAGCGACCTCCCGGTGAAGGTGATCCACGTGGAGAGTGGGAAGATCCTCAC
AGGCACAGATGCCCCCAAAGCCGGGCAGCTGGAGGCCTGGCTCGAGATGAACCCGGGGTATGAAGTAGCT
CCGAGGTCTGATAGTGAAGAAAGTGGCTCAGAAGAAGAGGAAGAGGAGGAGGAGGAAGAGCAGCCGCAGG
CAGCACAGCCTCCCACCCTGCCCGTGGAGGAGAAGAAGAAGATTCCAGATCCAGACAGCGATGACGTCTC
TGAGGTGGACGCGCGGCACATCATTGAGAATGCCAAGCAAGATGTCGATGATGAATATGGCGTGTCCCAG
GCCCTTGCACGTGGCCTGCAGTCCTACTATGCCGTGGCCCATGCTGTCACTGAGAGAGTGGACAAGCAGT
CAGCGCTTATGGTCAATGGTGTCCTCAAACAGTACCAGATCAAAGGTTTGGAGTGGCTGGTGTCCCTGTA
CAACAACAACCTGAACGGCATCCTGGCCGACGAGATGGGCCTGGGGAAGACCATCCAGACCATCGCGCTC
ATCACGTACCTCATGGAGCACAAACGCATCAATGGGCCCTTCCTCATCATCGTGCCTCTCTCAACGCTGT
CCAACTGGGCGTACGAGTTTGACAAGTGGGCCCCCTCCGTGGTGAAGGTGTCTTACAAGGGATCCCCAGC
AGCAAGACGGGCCTTTGTCCCCCAGCTCCGGAGTGGGAAGTTCAACGTCTTGCTGACGACGTACGAGTAC
ATCATCAAAGACAAGCACATCCTCGCCAAGATCCGTTGGAAGTACATGATTGTGGACGAAGGTCACCGCA
TGAAGAACCACCACTGCAAGCTGACGCAGGTGCTCAACACGCACTATGTGGCACCCCGCCGCCTGCTGCT
GACGGGCACACCGCTGCAGAACAAGCTTCCCGAGCTCTGGGCGCTGCTCAACTTCCTGCTGCCCACCATC
TTCAAGAGCTGCAGCACCTTCGAGCAGTGGTTTAACGCACCCTTTGCCATGACCGGGGAAAAGGTGGACC
TGAATGAGGAGGAAACCATTCTCATCATCCGGCGTCTCCACAAAGTGCTGCGGCCCTTCTTGCTCCGACG
ACTCAAGAAGGAAGTCGAGGCCCAGTTGCCCGAAAAGGTGGAGTACGTCATCAAGTGCGACATGTCTGCG
CTGCAGCGAGTGCTCTACCGCCACATGCAGGCCAAGGGCGTGCTGCTGACTGATGGCTCCGAGAAGGACA
AGAAGGGCAAAGGCGGCACCAAGACCCTGATGAACACCATCATGCAGCTGCGGAAGATCTGCAACCACCC
CTACATGTTCCAGCACATCGAGGAGTCCTTTTCCGAGCACTTGGGGTTCACTGGCGGCATTGTCCAAGGG
CTGGACCTGTACCGAGCCTCGGGTAAATTTGAGCTTCTTGATAGAATTCTTCCCAAACTCCGAGCAACCA
ACCACAAAGTGCTGCTGTTCTGCCAAATGACCTCCCTCATGACCATCATGGAAGATTACTTTGCGTATCG
CGGCTTTAAATACCTCAGGCTTGATGGAACCACGAAGGCGGAGGACCGGGGCATGCTGCTGAAAACCTTC
AACGAGCCCGGCTCTGAGTACTTCATCTTCCTGCTCAGCACCCGGGCTGGGGGGCTCGGCCTGAACCTCC
AGTCGGCAGACACTGTGATCATTTTTGACAGCGACTGGAATCCTCACCAGGACCTGCAAGCGCAGGACCG
AGCCCACCGCATCGGGCAGCAGAACGAGGTGCGTGTGCTCCGCCTCTGCACCGTCAACAGCGTGGAGGAG
AAGATCCTAGCTGCAGCCAAGTACAAGCTCAACGTGGACCAGAAGGTGATCCAGGCCGGCATGTTCGACC
AGAAGTCCTCCAGCCATGAGCGGCGCGCCTTCCTGCAGGCCATCCTGGAGCACGAGGAGCAGGATGAGAG
CAGACACTGCAGCACGGGCAGCGGCAGTGCCAGCTTCGCCCACACTGCCCCTCCGCCAGCGGGCGTCAAC
CCCGACTTGGAGGAGCCACCTCTAAAGGAGGAAGACGAGGTGCCCGACGACGAGACCGTCAACCAGATGA
TCGCCCGGCACGAGGAGGAGTTTGATCTGTTCATGCGCATGGACCTGGACCGCAGGCGCGAGGAGGCCCG
CAACCCCAAGCGGAAGCCGCGCCTCATGGAGGAGGACGAGCTCCCCTCGTGGATCATCAAGGACGACGCG
GAGGTGGAGCGGCTGACCTGTGAGGAGGAGGAGGAGAAGATGTTCGGCCGTGGCTCCCGCCACCGCAAGG
AGGTGGACTACAGCGACTCACTGACGGAGAAGCAGTGGCTCAAGAAAATTACAGGAAAAGATATCCATGA
CACAGCCAGCAGTGTGGCACGTGGGCTACAATTCCAGCGTGGCCTTCAGTTCTGCACACGTGCGTCAAAG
GCCATCGAGGAGGGCACGCTGGAGGAGATCGAAGAGGAGGTCCGGCAGAAGAAATCATCACGGAAGCGCA
AGCGAGACAGCGACGCCGGCTCCTCCACCCCGACCACCAGCACCCGCAGCCGCGACAAGGACGACGAGAG
CAAGAAGCAGAAGAAGCGCGGGCGGCCGCCTGCCGAGAAACTCTCCCCTAACCCACCCAACCTCACCAAG
AAGATGAAGAAGATTGTGGATGCCGTGATCAAGTACAAGGACAGCAGCAGTGGACGTCAGCTCAGCGAGG
TCTTCATCCAGCTGCCCTCGCGAAAGGAGCTGCCCGAGTACTACGAGCTCATCCGCAAGCCCGTGGACTT
CAAGAAGATAAAGGAGCGCATTCGCAACCACAAGTACCGCAGCCTCAACGACCTAGAGAAGGACGTCATG
CTCCTGTGCCAGAACGCACAGACCTTCAACCTGGAGGGCTCCCTGATCTATGAAGACTCCATCGTCTTGC
AGTCGGTCTTCACCAGCGTGCGGCAGAAAATCGAGAAGGAGGATGACAGTGAAGGCGAGGAGAGTGAGGA
GGAGGAAGAGGGCGAGGAGGAAGGCTCCGAATCCGAATCTCGGTCCGTCAAAGTGAAGATCAAGCTTGGC
CGGAAGGAGAAGGCACAGGACCGGCTGAAGGGCGGCCGGCGGCGGCCGAGCCGAGGGTCCCGAGCCAAGC
CGGTCGTGAGTGACGATGACAGTGAGGAGGAACAAGAGGAGGACCGCTCAGGAAGTGGCAGCGAAGAAGA
CTGAGCCCCGACATTCCAGTCTCGACCCCGAGCCCCTCGTTCCAGAGCTGAGATGGCATAGGCCTTAGCA
GTAACGGGTAGCAGCAGATGTAGTTTCAGACTTGGAGTAAAACTGTATAAACAAAAGAATCTTCCATATT
TATACAGCAGAGAAGCTGTAGGACTGTTTGTGACTGGCCCTGTCCTGGCATCAGTAGCATCTGTAACAGC
ATTAACTGTCTTAAAGAGAGAGAGAGAGAATTCCGAATTGGGGAACACACGATACCTGTTTTTCTTTTCC
GTTGCTGGCAGTACTGTTGCGCCGCAGTTTGGAGTCACTGTAGTTAAGTGTGGATGCATGTGCGTCACCG
TCCACTCCTCCTACTGTATTTTATTGGACAGGTCAGACTCGCCGGGGGCCCGGCGAGGGTATGTCAGTGT
CACTGGATGTCAAACAGTAATAAATTAAACCAACAACAAAACGCACAGCCAAAAAAAAA
mRNA sequence of human SWI/SNF related, matrix associated, actin
dependent regulator of chromatin, subfamily a, member 4 (SMARCA4),
transcript variant 2 (GenBank Accession No. NM_001128844.1)
(SEQ ID NO: 10)
GGAGAGGCCGCCGCGGTGCTGAGGGGGAGGGGAGCCGGCGAGCGCGCGCGCAGCGGGGGCGCGGGTGGCG
CGCGTGTGTGTGAAGGGGGGGCGGTGGCCGAGGCGGGCGGGCGCGCGCGCGAGGCTTCCCCTCGTTTGGC
GGCGGCGGCGGCTTCTTTGTTTCGTGAAGAGAAGCGAGACGCCCATTCTGCCCCCGGCCCCGCGCGGAGG
GGCGGGGGAGGCGCCGGGAAGTCGACGGCGCCGGCGGCTCCTGCGTCTCGCCCTTTTGCCCAGGCTAGAG
TGCAGTGGTGCGGTCATGGTTCACTGCAGCCTCAACCTCCTGGACTCAGCAGGAGGCCACTGTCTGCAGC
TCCCGTGAAGATGTCCACTCCAGACCCACCCCTGGGCGGAACTCCTCGGCCAGGTCCTTCCCCGGGCCCT
GGCCCTTCCCCTGGAGCCATGCTGGGCCCTAGCCCGGGTCCCTCGCCGGGCTCCGCCCACAGCATGATGG
GGCCCAGCCCAGGGCCGCCCTCAGCAGGACACCCCATCCCCACCCAGGGGCCTGGAGGGTACCCTCAGGA
CAACATGCACCAGATGCACAAGCCCATGGAGTCCATGCATGAGAAGGGCATGTCGGACGACCCGCGCTAC
AACCAGATGAAAGGAATGGGGATGCGGTCAGGGGGCCATGCTGGGATGGGGCCCCCGCCCAGCCCCATGG
ACCAGCACTCCCAAGGTTACCCCTCGCCCCTGGGTGGCTCTGAGCATGCCTCTAGTCCAGTTCCAGCCAG
TGGCCCGTCTTCGGGGCCCCAGATGTCTTCCGGGCCAGGAGGTGCCCCGCTGGATGGTGCTGACCCCCAG
GCCTTGGGGCAGCAGAACCGGGGCCCAACCCCATTTAACCAGAACCAGCTGCACCAGCTCAGAGCTCAGA
TCATGGCCTACAAGATGCTGGCCAGGGGGCAGCCCCTCCCCGACCACCTGCAGATGGCGGTGCAGGGCAA
GCGGCCGATGCCCGGGATGCAGCAGCAGATGCCAACGCTACCTCCACCCTCGGTGTCCGCAACAGGACCC
GGCCCTGGCCCTGGCCCTGGCCCCGGCCCGGGTCCCGGCCCGGCACCTCCAAATTACAGCAGGCCTCATG
GTATGGGAGGGCCCAACATGCCTCCCCCAGGACCCTCGGGCGTGCCCCCCGGGATGCCAGGCCAGCCTCC
TGGAGGGCCTCCCAAGCCCTGGCCTGAAGGACCCATGGCGAATGCTGCTGCCCCCACGAGCACCCCTCAG
AAGCTGATTCCCCCGCAGCCAACGGGCCGCCCTTCCCCCGCGCCCCCTGCCGTCCCACCCGCCGCCTCGC
CCGTGATGCCACCGCAGACCCAGTCCCCCGGGCAGCCGGCCCAGCCCGCGCCCATGGTGCCACTGCACCA
GAAGCAGAGCCGCATCACCCCCATCCAGAAGCCGCGGGGCCTCGACCCTGTGGAGATCCTGCAGGAGCGC
GAGTACAGGCTGCAGGCTCGCATCGCACACCGAATTCAGGAACTTGAAAACCTTCCCGGGTCCCTGGCCG
GGGATTTGCGAACCAAAGCGACCATTGAGCTCAAGGCCCTCAGGCTGCTGAACTTCCAGAGGCAGCTGCG
CCAGGAGGTGGTGGTGTGCATGCGGAGGGACACAGCGCTGGAGACAGCCCTCAATGCTAAGGCCTACAAG
CGCAGCAAGCGCCAGTCCCTGCGCGAGGCCCGCATCACTGAGAAGCTGGAGAAGCAGCAGAAGATCGAGC
AGGAGCGCAAGCGCCGGCAGAAGCACCAGGAATACCTCAATAGCATTCTCCAGCATGCCAAGGATTTCAA
GGAATATCACAGATCCGTCACAGGCAAAATCCAGAAGCTGACCAAGGCAGTGGCCACGTACCATGCCAAC
ACGGAGCGGGAGCAGAAGAAAGAGAACGAGCGGATCGAGAAGGAGCGCATGCGGAGGCTCATGGCTGAAG
ATGAGGAGGGGTACCGCAAGCTCATCGACCAGAAGAAGGACAAGCGCCTGGCCTACCTCTTGCAGCAGAC
AGACGAGTACGTGGCTAACCTCACGGAGCTGGTGCGGCAGCACAAGGCTGCCCAGGTCGCCAAGGAGAAA
AAGAAGAAAAAGAAAAAGAAGAAGGCAGAAAATGCAGAAGGACAGACGCCTGCCATTGGGCCGGATGGCG
AGCCTCTGGACGAGACCAGCCAGATGAGCGACCTCCCGGTGAAGGTGATCCACGTGGAGAGTGGGAAGAT
CCTCACAGGCACAGATGCCCCCAAAGCCGGGCAGCTGGAGGCCTGGCTCGAGATGAACCCGGGGTATGAA
GTAGCTCCGAGGTCTGATAGTGAAGAAAGTGGCTCAGAAGAAGAGGAAGAGGAGGAGGAGGAAGAGCAGC
CGCAGGCAGCACAGCCTCCCACCCTGCCCGTGGAGGAGAAGAAGAAGATTCCAGATCCAGACAGCGATGA
CGTCTCTGAGGTGGACGCGCGGCACATCATTGAGAATGCCAAGCAAGATGTCGATGATGAATATGGCGTG
TCCCAGGCCCTTGCACGTGGCCTGCAGTCCTACTATGCCGTGGCCCATGCTGTCACTGAGAGAGTGGACA
AGCAGTCAGCGCTTATGGTCAATGGTGTCCTCAAACAGTACCAGATCAAAGGTTTGGAGTGGCTGGTGTC
CCTGTACAACAACAACCTGAACGGCATCCTGGCCGACGAGATGGGCCTGGGGAAGACCATCCAGACCATC
GCGCTCATCACGTACCTCATGGAGCACAAACGCATCAATGGGCCCTTCCTCATCATCGTGCCTCTCTCAA
CGCTGTCCAACTGGGCGTACGAGTTTGACAAGTGGGCCCCCTCCGTGGTGAAGGTGTCTTACAAGGGATC
CCCAGCAGCAAGACGGGCCTTTGTCCCCCAGCTCCGGAGTGGGAAGTTCAACGTCTTGCTGACGACGTAC
GAGTACATCATCAAAGACAAGCACATCCTCGCCAAGATCCGTTGGAAGTACATGATTGTGGACGAAGGTC
ACCGCATGAAGAACCACCACTGCAAGCTGACGCAGGTGCTCAACACGCACTATGTGGCACCCCGCCGCCT
GCTGCTGACGGGCACACCGCTGCAGAACAAGCTTCCCGAGCTCTGGGCGCTGCTCAACTTCCTGCTGCCC
ACCATCTTCAAGAGCTGCAGCACCTTCGAGCAGTGGTTTAACGCACCCTTTGCCATGACCGGGGAAAAGG
TGGACCTGAATGAGGAGGAAACCATTCTCATCATCCGGCGTCTCCACAAAGTGCTGCGGCCCTTCTTGCT
CCGACGACTCAAGAAGGAAGTCGAGGCCCAGTTGCCCGAAAAGGTGGAGTACGTCATCAAGTGCGACATG
TCTGCGCTGCAGCGAGTGCTCTACCGCCACATGCAGGCCAAGGGCGTGCTGCTGACTGATGGCTCCGAGA
AGGACAAGAAGGGCAAAGGCGGCACCAAGACCCTGATGAACACCATCATGCAGCTGCGGAAGATCTGCAA
CCACCCCTACATGTTCCAGCACATCGAGGAGTCCTTTTCCGAGCACTTGGGGTTCACTGGCGGCATTGTC
CAAGGGCTGGACCTGTACCGAGCCTCGGGTAAATTTGAGCTTCTTGATAGAATTCTTCCCAAACTCCGAG
CAACCAACCACAAAGTGCTGCTGTTCTGCCAAATGACCTCCCTCATGACCATCATGGAAGATTACTTTGC
GTATCGCGGCTTTAAATACCTCAGGCTTGATGGAACCACGAAGGCGGAGGACCGGGGCATGCTGCTGAAA
ACCTTCAACGAGCCCGGCTCTGAGTACTTCATCTTCCTGCTCAGCACCCGGGCTGGGGGGCTCGGCCTGA
ACCTCCAGTCGGCAGACACTGTGATCATTTTTGACAGCGACTGGAATCCTCACCAGGACCTGCAAGCGCA
GGACCGAGCCCACCGCATCGGGCAGCAGAACGAGGTGCGTGTGCTCCGCCTCTGCACCGTCAACAGCGTG
GAGGAGAAGATCCTAGCTGCAGCCAAGTACAAGCTCAACGTGGACCAGAAGGTGATCCAGGCCGGCATGT
TCGACCAGAAGTCCTCCAGCCATGAGCGGCGCGCCTTCCTGCAGGCCATCCTGGAGCACGAGGAGCAGGA
TGAGAGCAGACACTGCAGCACGGGCAGCGGCAGTGCCAGCTTCGCCCACACTGCCCCTCCGCCAGCGGGC
GTCAACCCCGACTTGGAGGAGCCACCTCTAAAGGAGGAAGACGAGGTGCCCGACGACGAGACCGTCAACC
AGATGATCGCCCGGCACGAGGAGGAGTTTGATCTGTTCATGCGCATGGACCTGGACCGCAGGCGCGAGGA
GGCCCGCAACCCCAAGCGGAAGCCGCGCCTCATGGAGGAGGACGAGCTCCCCTCGTGGATCATCAAGGAC
GACGCGGAGGTGGAGCGGCTGACCTGTGAGGAGGAGGAGGAGAAGATGTTCGGCCGTGGCTCCCGCCACC
GCAAGGAGGTGGACTACAGCGACTCACTGACGGAGAAGCAGTGGCTCAAGGCCATCGAGGAGGGCACGCT
GGAGGAGATCGAAGAGGAGGTCCGGCAGAAGAAATCATCACGGAAGCGCAAGCGAGACAGCGACGCCGGC
TCCTCCACCCCGACCACCAGCACCCGCAGCCGCGACAAGGACGACGAGAGCAAGAAGCAGAAGAAGCGCG
GGCGGCCGCCTGCCGAGAAACTCTCCCCTAACCCACCCAACCTCACCAAGAAGATGAAGAAGATTGTGGA
TGCCGTGATCAAGTACAAGGACAGCAGCAGTGGACGTCAGCTCAGCGAGGTCTTCATCCAGCTGCCCTCG
CGAAAGGAGCTGCCCGAGTACTACGAGCTCATCCGCAAGCCCGTGGACTTCAAGAAGATAAAGGAGCGCA
TTCGCAACCACAAGTACCGCAGCCTCAACGACCTAGAGAAGGACGTCATGCTCCTGTGCCAGAACGCACA
GACCTTCAACCTGGAGGGCTCCCTGATCTATGAAGACTCCATCGTCTTGCAGTCGGTCTTCACCAGCGTG
CGGCAGAAAATCGAGAAGGAGGATGACAGTGAAGGCGAGGAGAGTGAGGAGGAGGAAGAGGGCGAGGAGG
AAGGCTCCGAATCCGAATCTCGGTCCGTCAAAGTGAAGATCAAGCTTGGCCGGAAGGAGAAGGCACAGGA
CCGGCTGAAGGGCGGCCGGCGGCGGCCGAGCCGAGGGTCCCGAGCCAAGCCGGTCGTGAGTGACGATGAC
AGTGAGGAGGAACAAGAGGAGGACCGCTCAGGAAGTGGCAGCGAAGAAGACTGAGCCCCGACATTCCAGT
CTCGACCCCGAGCCCCTCGTTCCAGAGCTGAGATGGCATAGGCCTTAGCAGTAACGGGTAGCAGCAGATG
TAGTTTCAGACTTGGAGTAAAACTGTATAAACAAAAGAATCTTCCATATTTATACAGCAGAGAAGCTGTA
GGACTGTTTGTGACTGGCCCTGTCCTGGCATCAGTAGCATCTGTAACAGCATTAACTGTCTTAAAGAGAG
AGAGAGAGAATTCCGAATTGGGGAACACACGATACCTGTTTTTCTTTTCCGTTGCTGGCAGTACTGTTGC
GCCGCAGTTTGGAGTCACTGTAGTTAAGTGTGGATGCATGTGCGTCACCGTCCACTCCTCCTACTGTATT
TTATTGGACAGGTCAGACTCGCCGGGGGCCCGGCGAGGGTATGTCAGTGTCACTGGATGTCAAACAGTAA
TAAATTAAACCAACAACAAAACGCACAGCCAAAAAAAAA
mRNA sequence of human SWI/SNF related, matrix associated, actin
dependent regulator of chromatin, subfamily a, member 4 (SMARCA4),
transcript variant 4 (GenBank Accession No. NM_001128845.1)
(SEQ ID NO: 11)
ATGTCCACTCCAGACCCACCCCTGGGCGGAACTCCTCGGCCAGGTCCTTCCCCGGGCCCTGGCCCTTCCC
CTGGAGCCATGCTGGGCCCTAGCCCGGGTCCCTCGCCGGGCTCCGCCCACAGCATGATGGGGCCCAGCCC
AGGGCCGCCCTCAGCAGGACACCCCATCCCCACCCAGGGGCCTGGAGGGTACCCTCAGGACAACATGCAC
CAGATGCACAAGCCCATGGAGTCCATGCATGAGAAGGGCATGTCGGACGACCCGCGCTACAACCAGATGA
AAGGAATGGGGATGCGGTCAGGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAGTTCCAGCCAGTGGCCCGTCT
TCGGGGCCCCAGATGTCTTCCGGGCCAGGAGGTGCCCCGCTGGATGGTGCTGACCCCCAGGCCTTGGGGC
AGCAGAACCGGGGCCCAACCCCATTTAACCAGAACCAGCTGCACCAGCTCAGAGCTCAGATCATGGCCTA
CAAGATGCTGGCCAGGGGGCAGCCCCTCCCCGACCACCTGCAGATGGCGGTGCAGGGCAAGCGGCCGATG
CCCGGGATGCAGCAGCAGATGCCAACGCTACCTCCACCCTCGGTGTCCGCAACAGGACCCGGCCCTGGCC
CTGGCCCTGGCCCCGGCCCGGGTCCCGGCCCGGCACCTCCAAATTACAGCAGGCCTCATGGTATGGGAGG
GCCCAACATGCCTCCCCCAGGACCCTCGGGCGTGCCCCCCGGGATGCCAGGCCAGCCTCCTGGAGGGCCT
CCCAAGCCCTGGCCTGAAGGACCCATGGCGAATGCTGCTGCCCCCACGAGCACCCCTCAGAAGCTGATTC
CCCCGCAGCCAACGGGCCGCCCTTCCCCCGCGCCCCCTGCCGTCCCACCCGCCGCCTCGCCCGTGATGCC
ACCGCAGACCCAGTCCCCCGGGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCTGCAGGAGCGCGAGTACAGGC
TGCAGGCTCGCATCGCACACCGAATTCAGGAACTTGAAAACCTTCCCGGGTCCCTGGCCGGGGATTTGCG
AACCAAAGCGACCATTGAGCTCAAGGCCCTCAGGCTGCTGAACTTCCAGAGGCAGCTGCGCCAGGAGGTG
GTGGTGTGCATGCGGAGGGACACAGCGCTGGAGACAGCCCTCAATGCTAAGGCCTACAAGCGCAGCAAGC
GCCAGTCCCTGCGCGAGGCCCGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCAAGGATTTCAAGGAATATCAC
AGATCCGTCACAGGCAAAATCCAGAAGCTGACCAAGGCAGTGGCCACGTACCATGCCAACACGGAGCGGG
AGCAGAAGAAAGAGAACGAGCGGATCGAGAAGGAGCGCATGCGGAGGCTCATGGCTGAAGATGAGGAGGG
GTACCGCAAGCTCATCGACCAGAAGAAGGACAAGCGCCTGGCCTACCTCTTGCAGCAGACAGACGAGTAC
GTGGCTAACCTCACGGAGCTGGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGGCCGGATGGCGAGCCTCTGGA
CGAGACCAGCCAGATGAGCGACCTCCCGGTGAAGGTGATCCACGTGGAGAGTGGGAAGATCCTCACAGGC
ACAGATGCCCCCAAAGCCGGGCAGCTGGAGGCCTGGCTCGAGATGAACCCGGGGTATGAAGTAGCTCCGA
GGTCTGATAGTGAAGAAAGTGGCTCAGAAGAAGAGGAAGAGGAGGAGGAGGAAGAGCAGCCGCAGGCAGC
ACAGCCTCCCACCCTGCCCGTGGAGGAGAAGAAGAAGATTCCAGATCCAGACAGCGATGACGTCTCTGAG
GTGGACGCGCGGCACATCATTGAGAATGCCAAGCAAGATGTCGATGATGAATATGGCGTGTCCCAGGCCC
TTGCACGTGGCCTGCAGTCCTACTATGCCGTGGCCCATGCTGTCACTGAGAGAGTGGACAAGCAGTCAGC
GCTTATGGTCAATGGTGTCCTCAAACAGTACCAGATCAAAGGTTTGGAGTGGCTGGTGTCCCTGTACAAC
AACAACCTGAACGGCATCCTGGCCGACGAGATGGGCCTGGGGAAGACCATCCAGACCATCGCGCTCATCA
CGTACCTCATGGAGCACAAACGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTTACAAGGGATCCCCAGCAGCA
AGACGGGCCTTTGTCCCCCAGCTCCGGAGTGGGAAGTTCAACGTCTTGCTGACGACGTACGAGTACATCA
TCAAAGACAAGCACATCCTCGCCAAGATCCGTTGGAAGTACATGATTGTGGACGAAGGTCACCGCATGAA
GAACCACCACTGCAAGCTGACGCAGGTGCTCAACACGCACTATGTGGCACCCCGCCGCCTGCTGCTGACG
GGCACACCGCTGCAGAACAAGCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACCGGGGAAAAGGTGGACCTGAA
TGAGGAGGAAACCATTCTCATCATCCGGCGTCTCCACAAAGTGCTGCGGCCCTTCTTGCTCCGACGACTC
AAGAAGGAAGTCGAGGCCCAGTTGCCCGAAAAGGTGGAGTACGTCATCAAGTGCGACATGTCTGCGCTGC
AGCGAGTGCTCTACCGCCACATGCAGGCCAAGGGCGTGCTGCTGACTGATGGCTCCGAGAAGGACAAGAA
GGGCAAAGGCGGCACCAAGACCCTGATGAACACCATCATGCAGCTGCGGAAGATCTGCAACCACCCCTAC
ATGTTCCAGCACATCGAGGAGTCCTTTTCCGAGCACTTGGGGTTCACTGGCGGCATTGTCCAAGGGCTGG
ACCTGTACCGAGCCTCGGGTAAATTTGAGCTTCTTGATAGAATTCTTCCCAAACTCCGAGCAACCAACCA
CAAAGTGCTGCTGTTCTGCCAAATGACCTCCCTCATGACCATCATGGAAGATTACTTTGCGTATCGCGGC
TTTAAATACCTCAGGCTTGATGGAACCACGAAGGCGGAGGACCGGGGCATGCTGCTGAAAACCTTCAACG
AGCCCGGCTCTGAGTACTTCATCTTCCTGCTCAGCACCCGGGCTGGGGGGCTCGGCCTGAACCTCCAGTC
GGCAGACACTGTGATCATTTTTGACAGCGACTGGAATCCTCACCAGGACCTGCAAGCGCAGGACCGAGCC
CACCGCATCGGGCAGCAGAACGAGGTGCGTGTGCTCCGCCTCTGCACCGTCAACAGCGTGGAGGAGAAGA
TCCTAGCTGCAGCCAAGTACAAGCTCAACGTGGACCAGAAGGTGATCCAGGCCGGCATGTTCGACCAGAA
GTCCTCCAGCCATGAGCGGCGCGCCTTCCTGCAGGCCATCCTGGAGCACGAGGAGCAGGATGAGGAGGAA
GACGAGGTGCCCGACGACGAGACCGTCAACCAGATGATCGCCCGGCACGAGGAGGAGTTTGATCTGTTCA
TGCGCATGGACCTGGACCGCAGGCGCGAGGAGGCCCGCAACCCCAAGCGGAAGCCGCGCCTCATGGAGGA
GGACGAGCTCCCCTCGTGGATCATCAAGGACGACGCGGAGGTGGAGCGGCTGACCTGTGAGGAGGAGGAG
GAGAAGATGTTCGGCCGTGGCTCCCGCCACCGCAAGGAGGTGGACTACAGCGACTCACTGACGGAGAAGC
AGTGGCTCAAGACCCTGAAGGCCATCGAGGAGGGCACGCTGGAGGAGATCGAAGAGGAGGTCCGGCAGAA
GAAATCATCACGGAAGCGCAAGCGAGACAGCGACGCCGGCTCCTCCACCCCGACCACCAGCACCCGCAGC
CGCGACAAGGACGACGAGAGCAAGAAGCAGAAGAAGCGCGGGCGGCCGCCTGCCGAGAAACTCTCCCCTA
ACCCACCCAACCTCACCAAGAAGATGAAGAAGATTGTGGATGCCGTGATCAAGTACAAGGACAGCAGCAG
TGGACGTCAGCTCAGCGAGGTCTTCATCCAGCTGCCCTCGCGAAAGGAGCTGCCCGAGTACTACGAGCTC
ATCCGCAAGCCCGTGGACTTCAAGAAGATAAAGGAGCGCATTCGCAACCACAAGTACCGCAGCCTCAACG
ACCTAGAGAAGGACGTCATGCTCCTGTGCCAGAACGCACAGACCTTCAACCTGGAGGGCTCCCTGATCTA
TGAAGACTCCATCGTCTTGCAGTCGGTCTTCACCAGCGTGCGGCAGAAAATCGAGAAGGAGGATGACAGT
GAAGGCGAGGAGAGTGAGGAGGAGGAAGAGGGCGAGGAGGAAGGCTCCGAATCCGAATCTCGGTCCGTCA
AAGTGAAGATCAAGCTTGGCCGGAAGGAGAAGGCACAGGACCGGCTGAAGGGCGGCCGGCGGCGGCCGAG
CCGAGGGTCCCGAGCCAAGCCGGTCGTGAGTGACGATGACAGTGAGGAGGAACAAGAGGAGGACCGCTCA
GGAAGTGGCAGCGAAGAAGACTGAGCCCCGACATTCCAGTCTCGACCCCGAGCCCCTCGTTCCAGAGCTG
AGATGGCATAGGCCTTAGCAGTAACGGGTAGCAGCAGATGTAGTTTCAGACTTGGAGTAAAACTGTATAA
ACAAAAGAATCTTCCATATTTATACAGCAGAGAAGCTGTAGGACTGTTTGTGACTGGCCCTGTCCTGGCA
TCAGTAGCATCTGTAACAGCATTAACTGTCTTAAAGAGAGAGAGAGAGAATTCCGAATTGGGGAACACAC
GATACCTGTTTTTCTTTTCCGTTGCTGGCAGTACTGTTGCGCCGCAGTTTGGAGTCACTGTAGTTAAGTG
TGGATGCATGTGCGTCACCGTCCACTCCTCCTACTGTATTTTATTGGACAGGTCAGACTCGCCGGGGGCC
CGGCGAGGGTATGTCAGTGTCACTGGATGTCAAACAGTAATAAATTAAACCAACAACAAAACGCACAGCC
AAAAAAAAA
mRNA sequence of human SWI/SNF related, matrix associated, actin
dependent regulator of chromatin, subfamily a, member 4 (SMARCA4),
transcript variant 5 (GenBank Accession No. NM_001128846.1)
(SEQ ID NO: 12)
ATGTCCACTCCAGACCCACCCCTGGGCGGAACTCCTCGGCCAGGTCCTTCCCCGGGCCCTGGCCCTTCCC
CTGGAGCCATGCTGGGCCCTAGCCCGGGTCCCTCGCCGGGCTCCGCCCACAGCATGATGGGGCCCAGCCC
AGGGCCGCCCTCAGCAGGACACCCCATCCCCACCCAGGGGCCTGGAGGGTACCCTCAGGACAACATGCAC
CAGATGCACAAGCCCATGGAGTCCATGCATGAGAAGGGCATGTCGGACGACCCGCGCTACAACCAGATGA
AAGGAATGGGGATGCGGTCAGGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAGTTCCAGCCAGTGGCCCGTCT
TCGGGGCCCCAGATGTCTTCCGGGCCAGGAGGTGCCCCGCTGGATGGTGCTGACCCCCAGGCCTTGGGGC
AGCAGAACCGGGGCCCAACCCCATTTAACCAGAACCAGCTGCACCAGCTCAGAGCTCAGATCATGGCCTA
CAAGATGCTGGCCAGGGGGCAGCCCCTCCCCGACCACCTGCAGATGGCGGTGCAGGGCAAGCGGCCGATG
CCCGGGATGCAGCAGCAGATGCCAACGCTACCTCCACCCTCGGTGTCCGCAACAGGACCCGGCCCTGGCC
CTGGCCCTGGCCCCGGCCCGGGTCCCGGCCCGGCACCTCCAAATTACAGCAGGCCTCATGGTATGGGAGG
GCCCAACATGCCTCCCCCAGGACCCTCGGGCGTGCCCCCCGGGATGCCAGGCCAGCCTCCTGGAGGGCCT
CCCAAGCCCTGGCCTGAAGGACCCATGGCGAATGCTGCTGCCCCCACGAGCACCCCTCAGAAGCTGATTC
CCCCGCAGCCAACGGGCCGCCCTTCCCCCGCGCCCCCTGCCGTCCCACCCGCCGCCTCGCCCGTGATGCC
ACCGCAGACCCAGTCCCCCGGGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCTGCAGGAGCGCGAGTACAGGC
TGCAGGCTCGCATCGCACACCGAATTCAGGAACTTGAAAACCTTCCCGGGTCCCTGGCCGGGGATTTGCG
AACCAAAGCGACCATTGAGCTCAAGGCCCTCAGGCTGCTGAACTTCCAGAGGCAGCTGCGCCAGGAGGTG
GTGGTGTGCATGCGGAGGGACACAGCGCTGGAGACAGCCCTCAATGCTAAGGCCTACAAGCGCAGCAAGC
GCCAGTCCCTGCGCGAGGCCCGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCAAGGATTTCAAGGAATATCAC
AGATCCGTCACAGGCAAAATCCAGAAGCTGACCAAGGCAGTGGCCACGTACCATGCCAACACGGAGCGGG
AGCAGAAGAAAGAGAACGAGCGGATCGAGAAGGAGCGCATGCGGAGGCTCATGGCTGAAGATGAGGAGGG
GTACCGCAAGCTCATCGACCAGAAGAAGGACAAGCGCCTGGCCTACCTCTTGCAGCAGACAGACGAGTAC
GTGGCTAACCTCACGGAGCTGGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGGCCGGATGGCGAGCCTCTGGA
CGAGACCAGCCAGATGAGCGACCTCCCGGTGAAGGTGATCCACGTGGAGAGTGGGAAGATCCTCACAGGC
ACAGATGCCCCCAAAGCCGGGCAGCTGGAGGCCTGGCTCGAGATGAACCCGGGGTATGAAGTAGCTCCGA
GGTCTGATAGTGAAGAAAGTGGCTCAGAAGAAGAGGAAGAGGAGGAGGAGGAAGAGCAGCCGCAGGCAGC
ACAGCCTCCCACCCTGCCCGTGGAGGAGAAGAAGAAGATTCCAGATCCAGACAGCGATGACGTCTCTGAG
GTGGACGCGCGGCACATCATTGAGAATGCCAAGCAAGATGTCGATGATGAATATGGCGTGTCCCAGGCCC
TTGCACGTGGCCTGCAGTCCTACTATGCCGTGGCCCATGCTGTCACTGAGAGAGTGGACAAGCAGTCAGC
GCTTATGGTCAATGGTGTCCTCAAACAGTACCAGATCAAAGGTTTGGAGTGGCTGGTGTCCCTGTACAAC
AACAACCTGAACGGCATCCTGGCCGACGAGATGGGCCTGGGGAAGACCATCCAGACCATCGCGCTCATCA
CGTACCTCATGGAGCACAAACGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTTACAAGGGATCCCCAGCAGCA
AGACGGGCCTTTGTCCCCCAGCTCCGGAGTGGGAAGTTCAACGTCTTGCTGACGACGTACGAGTACATCA
TCAAAGACAAGCACATCCTCGCCAAGATCCGTTGGAAGTACATGATTGTGGACGAAGGTCACCGCATGAA
GAACCACCACTGCAAGCTGACGCAGGTGCTCAACACGCACTATGTGGCACCCCGCCGCCTGCTGCTGACG
GGCACACCGCTGCAGAACAAGCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACCGGGGAAAAGGTGGACCTGAA
TGAGGAGGAAACCATTCTCATCATCCGGCGTCTCCACAAAGTGCTGCGGCCCTTCTTGCTCCGACGACTC
AAGAAGGAAGTCGAGGCCCAGTTGCCCGAAAAGGTGGAGTACGTCATCAAGTGCGACATGTCTGCGCTGC
AGCGAGTGCTCTACCGCCACATGCAGGCCAAGGGCGTGCTGCTGACTGATGGCTCCGAGAAGGACAAGAA
GGGCAAAGGCGGCACCAAGACCCTGATGAACACCATCATGCAGCTGCGGAAGATCTGCAACCACCCCTAC
ATGTTCCAGCACATCGAGGAGTCCTTTTCCGAGCACTTGGGGTTCACTGGCGGCATTGTCCAAGGGCTGG
ACCTGTACCGAGCCTCGGGTAAATTTGAGCTTCTTGATAGAATTCTTCCCAAACTCCGAGCAACCAACCA
CAAAGTGCTGCTGTTCTGCCAAATGACCTCCCTCATGACCATCATGGAAGATTACTTTGCGTATCGCGGC
TTTAAATACCTCAGGCTTGATGGAACCACGAAGGCGGAGGACCGGGGCATGCTGCTGAAAACCTTCAACG
AGCCCGGCTCTGAGTACTTCATCTTCCTGCTCAGCACCCGGGCTGGGGGGCTCGGCCTGAACCTCCAGTC
GGCAGACACTGTGATCATTTTTGACAGCGACTGGAATCCTCACCAGGACCTGCAAGCGCAGGACCGAGCC
CACCGCATCGGGCAGCAGAACGAGGTGCGTGTGCTCCGCCTCTGCACCGTCAACAGCGTGGAGGAGAAGA
TCCTAGCTGCAGCCAAGTACAAGCTCAACGTGGACCAGAAGGTGATCCAGGCCGGCATGTTCGACCAGAA
GTCCTCCAGCCATGAGCGGCGCGCCTTCCTGCAGGCCATCCTGGAGCACGAGGAGCAGGATGAGGAGGAA
GACGAGGTGCCCGACGACGAGACCGTCAACCAGATGATCGCCCGGCACGAGGAGGAGTTTGATCTGTTCA
TGCGCATGGACCTGGACCGCAGGCGCGAGGAGGCCCGCAACCCCAAGCGGAAGCCGCGCCTCATGGAGGA
GGACGAGCTCCCCTCGTGGATCATCAAGGACGACGCGGAGGTGGAGCGGCTGACCTGTGAGGAGGAGGAG
GAGAAGATGTTCGGCCGTGGCTCCCGCCACCGCAAGGAGGTGGACTACAGCGACTCACTGACGGAGAAGC
AGTGGCTCAAGACCCTGAAGGCCATCGAGGAGGGCACGCTGGAGGAGATCGAAGAGGAGGTCCGGCAGAA
GAAATCATCACGGAAGCGCAAGCGAGACAGCGACGCCGGCTCCTCCACCCCGACCACCAGCACCCGCAGC
CGCGACAAGGACGACGAGAGCAAGAAGCAGAAGAAGCGCGGGCGGCCGCCTGCCGAGAAACTCTCCCCTA
ACCCACCCAACCTCACCAAGAAGATGAAGAAGATTGTGGATGCCGTGATCAAGTACAAGGACAGCAGTGG
ACGTCAGCTCAGCGAGGTCTTCATCCAGCTGCCCTCGCGAAAGGAGCTGCCCGAGTACTACGAGCTCATC
CGCAAGCCCGTGGACTTCAAGAAGATAAAGGAGCGCATTCGCAACCACAAGTACCGCAGCCTCAACGACC
TAGAGAAGGACGTCATGCTCCTGTGCCAGAACGCACAGACCTTCAACCTGGAGGGCTCCCTGATCTATGA
AGACTCCATCGTCTTGCAGTCGGTCTTCACCAGCGTGCGGCAGAAAATCGAGAAGGAGGATGACAGTGAA
GGCGAGGAGAGTGAGGAGGAGGAAGAGGGCGAGGAGGAAGGCTCCGAATCCGAATCTCGGTCCGTCAAAG
TGAAGATCAAGCTTGGCCGGAAGGAGAAGGCACAGGACCGGCTGAAGGGCGGCCGGCGGCGGCCGAGCCG
AGGGTCCCGAGCCAAGCCGGTCGTGAGTGACGATGACAGTGAGGAGGAACAAGAGGAGGACCGCTCAGGA
AGTGGCAGCGAAGAAGACTGAGCCCCGACATTCCAGTCTCGACCCCGAGCCCCTCGTTCCAGAGCTGAGA
TGGCATAGGCCTTAGCAGTAACGGGTAGCAGCAGATGTAGTTTCAGACTTGGAGTAAAACTGTATAAACA
AAAGAATCTTCCATATTTATACAGCAGAGAAGCTGTAGGACTGTTTGTGACTGGCCCTGTCCTGGCATCA
GTAGCATCTGTAACAGCATTAACTGTCTTAAAGAGAGAGAGAGAGAATTCCGAATTGGGGAACACACGAT
ACCTGTTTTTCTTTTCCGTTGCTGGCAGTACTGTTGCGCCGCAGTTTGGAGTCACTGTAGTTAAGTGTGG
ATGCATGTGCGTCACCGTCCACTCCTCCTACTGTATTTTATTGGACAGGTCAGACTCGCCGGGGGCCCGG
CGAGGGTATGTCAGTGTCACTGGATGTCAAACAGTAATAAATTAAACCAACAACAAAACGCACAGCCAAA
AAAAAA
mRNA sequence of human SWI/SNF related, matrix associated, actin
dependent regulator of chromatin, subfamily a, member 4 (SMARCA4),
transcript variant 6 (GenBank Accession No. NM_001128847.1)
(SEQ ID NO: 13)
ATGTCCACTCCAGACCCACCCCTGGGCGGAACTCCTCGGCCAGGTCCTTCCCCGGGCCCTGGCCCTTCCC
CTGGAGCCATGCTGGGCCCTAGCCCGGGTCCCTCGCCGGGCTCCGCCCACAGCATGATGGGGCCCAGCCC
AGGGCCGCCCTCAGCAGGACACCCCATCCCCACCCAGGGGCCTGGAGGGTACCCTCAGGACAACATGCAC
CAGATGCACAAGCCCATGGAGTCCATGCATGAGAAGGGCATGTCGGACGACCCGCGCTACAACCAGATGA
AAGGAATGGGGATGCGGTCAGGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAGTTCCAGCCAGTGGCCCGTCT
TCGGGGCCCCAGATGTCTTCCGGGCCAGGAGGTGCCCCGCTGGATGGTGCTGACCCCCAGGCCTTGGGGC
AGCAGAACCGGGGCCCAACCCCATTTAACCAGAACCAGCTGCACCAGCTCAGAGCTCAGATCATGGCCTA
CAAGATGCTGGCCAGGGGGCAGCCCCTCCCCGACCACCTGCAGATGGCGGTGCAGGGCAAGCGGCCGATG
CCCGGGATGCAGCAGCAGATGCCAACGCTACCTCCACCCTCGGTGTCCGCAACAGGACCCGGCCCTGGCC
CTGGCCCTGGCCCCGGCCCGGGTCCCGGCCCGGCACCTCCAAATTACAGCAGGCCTCATGGTATGGGAGG
GCCCAACATGCCTCCCCCAGGACCCTCGGGCGTGCCCCCCGGGATGCCAGGCCAGCCTCCTGGAGGGCCT
CCCAAGCCCTGGCCTGAAGGACCCATGGCGAATGCTGCTGCCCCCACGAGCACCCCTCAGAAGCTGATTC
CCCCGCAGCCAACGGGCCGCCCTTCCCCCGCGCCCCCTGCCGTCCCACCCGCCGCCTCGCCCGTGATGCC
ACCGCAGACCCAGTCCCCCGGGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCTGCAGGAGCGCGAGTACAGGC
TGCAGGCTCGCATCGCACACCGAATTCAGGAACTTGAAAACCTTCCCGGGTCCCTGGCCGGGGATTTGCG
AACCAAAGCGACCATTGAGCTCAAGGCCCTCAGGCTGCTGAACTTCCAGAGGCAGCTGCGCCAGGAGGTG
GTGGTGTGCATGCGGAGGGACACAGCGCTGGAGACAGCCCTCAATGCTAAGGCCTACAAGCGCAGCAAGC
GCCAGTCCCTGCGCGAGGCCCGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCAAGGATTTCAAGGAATATCAC
AGATCCGTCACAGGCAAAATCCAGAAGCTGACCAAGGCAGTGGCCACGTACCATGCCAACACGGAGCGGG
AGCAGAAGAAAGAGAACGAGCGGATCGAGAAGGAGCGCATGCGGAGGCTCATGGCTGAAGATGAGGAGGG
GTACCGCAAGCTCATCGACCAGAAGAAGGACAAGCGCCTGGCCTACCTCTTGCAGCAGACAGACGAGTAC
GTGGCTAACCTCACGGAGCTGGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGGCCGGATGGCGAGCCTCTGGA
CGAGACCAGCCAGATGAGCGACCTCCCGGTGAAGGTGATCCACGTGGAGAGTGGGAAGATCCTCACAGGC
ACAGATGCCCCCAAAGCCGGGCAGCTGGAGGCCTGGCTCGAGATGAACCCGGGGTATGAAGTAGCTCCGA
GGTCTGATAGTGAAGAAAGTGGCTCAGAAGAAGAGGAAGAGGAGGAGGAGGAAGAGCAGCCGCAGGCAGC
ACAGCCTCCCACCCTGCCCGTGGAGGAGAAGAAGAAGATTCCAGATCCAGACAGCGATGACGTCTCTGAG
GTGGACGCGCGGCACATCATTGAGAATGCCAAGCAAGATGTCGATGATGAATATGGCGTGTCCCAGGCCC
TTGCACGTGGCCTGCAGTCCTACTATGCCGTGGCCCATGCTGTCACTGAGAGAGTGGACAAGCAGTCAGC
GCTTATGGTCAATGGTGTCCTCAAACAGTACCAGATCAAAGGTTTGGAGTGGCTGGTGTCCCTGTACAAC
AACAACCTGAACGGCATCCTGGCCGACGAGATGGGCCTGGGGAAGACCATCCAGACCATCGCGCTCATCA
CGTACCTCATGGAGCACAAACGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTTACAAGGGATCCCCAGCAGCA
AGACGGGCCTTTGTCCCCCAGCTCCGGAGTGGGAAGTTCAACGTCTTGCTGACGACGTACGAGTACATCA
TCAAAGACAAGCACATCCTCGCCAAGATCCGTTGGAAGTACATGATTGTGGACGAAGGTCACCGCATGAA
GAACCACCACTGCAAGCTGACGCAGGTGCTCAACACGCACTATGTGGCACCCCGCCGCCTGCTGCTGACG
GGCACACCGCTGCAGAACAAGCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACCGGGGAAAAGGTGGACCTGAA
TGAGGAGGAAACCATTCTCATCATCCGGCGTCTCCACAAAGTGCTGCGGCCCTTCTTGCTCCGACGACTC
AAGAAGGAAGTCGAGGCCCAGTTGCCCGAAAAGGTGGAGTACGTCATCAAGTGCGACATGTCTGCGCTGC
AGCGAGTGCTCTACCGCCACATGCAGGCCAAGGGCGTGCTGCTGACTGATGGCTCCGAGAAGGACAAGAA
GGGCAAAGGCGGCACCAAGACCCTGATGAACACCATCATGCAGCTGCGGAAGATCTGCAACCACCCCTAC
ATGTTCCAGCACATCGAGGAGTCCTTTTCCGAGCACTTGGGGTTCACTGGCGGCATTGTCCAAGGGCTGG
ACCTGTACCGAGCCTCGGGTAAATTTGAGCTTCTTGATAGAATTCTTCCCAAACTCCGAGCAACCAACCA
CAAAGTGCTGCTGTTCTGCCAAATGACCTCCCTCATGACCATCATGGAAGATTACTTTGCGTATCGCGGC
TTTAAATACCTCAGGCTTGATGGAACCACGAAGGCGGAGGACCGGGGCATGCTGCTGAAAACCTTCAACG
AGCCCGGCTCTGAGTACTTCATCTTCCTGCTCAGCACCCGGGCTGGGGGGCTCGGCCTGAACCTCCAGTC
GGCAGACACTGTGATCATTTTTGACAGCGACTGGAATCCTCACCAGGACCTGCAAGCGCAGGACCGAGCC
CACCGCATCGGGCAGCAGAACGAGGTGCGTGTGCTCCGCCTCTGCACCGTCAACAGCGTGGAGGAGAAGA
TCCTAGCTGCAGCCAAGTACAAGCTCAACGTGGACCAGAAGGTGATCCAGGCCGGCATGTTCGACCAGAA
GTCCTCCAGCCATGAGCGGCGCGCCTTCCTGCAGGCCATCCTGGAGCACGAGGAGCAGGATGAGGAGGAA
GACGAGGTGCCCGACGACGAGACCGTCAACCAGATGATCGCCCGGCACGAGGAGGAGTTTGATCTGTTCA
TGCGCATGGACCTGGACCGCAGGCGCGAGGAGGCCCGCAACCCCAAGCGGAAGCCGCGCCTCATGGAGGA
GGACGAGCTCCCCTCGTGGATCATCAAGGACGACGCGGAGGTGGAGCGGCTGACCTGTGAGGAGGAGGAG
GAGAAGATGTTCGGCCGTGGCTCCCGCCACCGCAAGGAGGTGGACTACAGCGACTCACTGACGGAGAAGC
AGTGGCTCAAGGCCATCGAGGAGGGCACGCTGGAGGAGATCGAAGAGGAGGTCCGGCAGAAGAAATCATC
ACGGAAGCGCAAGCGAGACAGCGACGCCGGCTCCTCCACCCCGACCACCAGCACCCGCAGCCGCGACAAG
GACGACGAGAGCAAGAAGCAGAAGAAGCGCGGGCGGCCGCCTGCCGAGAAACTCTCCCCTAACCCACCCA
ACCTCACCAAGAAGATGAAGAAGATTGTGGATGCCGTGATCAAGTACAAGGACAGCAGCAGTGGACGTCA
GCTCAGCGAGGTCTTCATCCAGCTGCCCTCGCGAAAGGAGCTGCCCGAGTACTACGAGCTCATCCGCAAG
CCCGTGGACTTCAAGAAGATAAAGGAGCGCATTCGCAACCACAAGTACCGCAGCCTCAACGACCTAGAGA
AGGACGTCATGCTCCTGTGCCAGAACGCACAGACCTTCAACCTGGAGGGCTCCCTGATCTATGAAGACTC
CATCGTCTTGCAGTCGGTCTTCACCAGCGTGCGGCAGAAAATCGAGAAGGAGGATGACAGTGAAGGCGAG
GAGAGTGAGGAGGAGGAAGAGGGCGAGGAGGAAGGCTCCGAATCCGAATCTCGGTCCGTCAAAGTGAAGA
TCAAGCTTGGCCGGAAGGAGAAGGCACAGGACCGGCTGAAGGGCGGCCGGCGGCGGCCGAGCCGAGGGTC
CCGAGCCAAGCCGGTCGTGAGTGACGATGACAGTGAGGAGGAACAAGAGGAGGACCGCTCAGGAAGTGGC
AGCGAAGAAGACTGAGCCCCGACATTCCAGTCTCGACCCCGAGCCCCTCGTTCCAGAGCTGAGATGGCAT
AGGCCTTAGCAGTAACGGGTAGCAGCAGATGTAGTTTCAGACTTGGAGTAAAACTGTATAAACAAAAGAA
TCTTCCATATTTATACAGCAGAGAAGCTGTAGGACTGTTTGTGACTGGCCCTGTCCTGGCATCAGTAGCA
TCTGTAACAGCATTAACTGTCTTAAAGAGAGAGAGAGAGAATTCCGAATTGGGGAACACACGATACCTGT
TTTTCTTTTCCGTTGCTGGCAGTACTGTTGCGCCGCAGTTTGGAGTCACTGTAGTTAAGTGTGGATGCAT
GTGCGTCACCGTCCACTCCTCCTACTGTATTTTATTGGACAGGTCAGACTCGCCGGGGGCCCGGCGAGGG
TATGTCAGTGTCACTGGATGTCAAACAGTAATAAATTAAACCAACAACAAAACGCACAGCCAAAAAAAAA
mRNA sequence of human SWI/SNF related, matrix associated, actin
dependent regulator of chromatin, subfamily a, member 4 (SMARCA4),
transcript variant 7 (GenBank Accession No. NM_001128848.1)
(SEQ ID NO: 14)
ATGTCCACTCCAGACCCACCCCTGGGCGGAACTCCTCGGCCAGGTCCTTCCCCGGGCCCTGGCCCTTCCC
CTGGAGCCATGCTGGGCCCTAGCCCGGGTCCCTCGCCGGGCTCCGCCCACAGCATGATGGGGCCCAGCCC
AGGGCCGCCCTCAGCAGGACACCCCATCCCCACCCAGGGGCCTGGAGGGTACCCTCAGGACAACATGCAC
CAGATGCACAAGCCCATGGAGTCCATGCATGAGAAGGGCATGTCGGACGACCCGCGCTACAACCAGATGA
AAGGAATGGGGATGCGGTCAGGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAGTTCCAGCCAGTGGCCCGTCT
TCGGGGCCCCAGATGTCTTCCGGGCCAGGAGGTGCCCCGCTGGATGGTGCTGACCCCCAGGCCTTGGGGC
AGCAGAACCGGGGCCCAACCCCATTTAACCAGAACCAGCTGCACCAGCTCAGAGCTCAGATCATGGCCTA
CAAGATGCTGGCCAGGGGGCAGCCCCTCCCCGACCACCTGCAGATGGCGGTGCAGGGCAAGCGGCCGATG
CCCGGGATGCAGCAGCAGATGCCAACGCTACCTCCACCCTCGGTGTCCGCAACAGGACCCGGCCCTGGCC
CTGGCCCTGGCCCCGGCCCGGGTCCCGGCCCGGCACCTCCAAATTACAGCAGGCCTCATGGTATGGGAGG
GCCCAACATGCCTCCCCCAGGACCCTCGGGCGTGCCCCCCGGGATGCCAGGCCAGCCTCCTGGAGGGCCT
CCCAAGCCCTGGCCTGAAGGACCCATGGCGAATGCTGCTGCCCCCACGAGCACCCCTCAGAAGCTGATTC
CCCCGCAGCCAACGGGCCGCCCTTCCCCCGCGCCCCCTGCCGTCCCACCCGCCGCCTCGCCCGTGATGCC
ACCGCAGACCCAGTCCCCCGGGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCTGCAGGAGCGCGAGTACAGGC
TGCAGGCTCGCATCGCACACCGAATTCAGGAACTTGAAAACCTTCCCGGGTCCCTGGCCGGGGATTTGCG
AACCAAAGCGACCATTGAGCTCAAGGCCCTCAGGCTGCTGAACTTCCAGAGGCAGCTGCGCCAGGAGGTG
GTGGTGTGCATGCGGAGGGACACAGCGCTGGAGACAGCCCTCAATGCTAAGGCCTACAAGCGCAGCAAGC
GCCAGTCCCTGCGCGAGGCCCGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCAAGGATTTCAAGGAATATCAC
AGATCCGTCACAGGCAAAATCCAGAAGCTGACCAAGGCAGTGGCCACGTACCATGCCAACACGGAGCGGG
AGCAGAAGAAAGAGAACGAGCGGATCGAGAAGGAGCGCATGCGGAGGCTCATGGCTGAAGATGAGGAGGG
GTACCGCAAGCTCATCGACCAGAAGAAGGACAAGCGCCTGGCCTACCTCTTGCAGCAGACAGACGAGTAC
GTGGCTAACCTCACGGAGCTGGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGGCCGGATGGCGAGCCTCTGGA
CGAGACCAGCCAGATGAGCGACCTCCCGGTGAAGGTGATCCACGTGGAGAGTGGGAAGATCCTCACAGGC
ACAGATGCCCCCAAAGCCGGGCAGCTGGAGGCCTGGCTCGAGATGAACCCGGGGTATGAAGTAGCTCCGA
GGTCTGATAGTGAAGAAAGTGGCTCAGAAGAAGAGGAAGAGGAGGAGGAGGAAGAGCAGCCGCAGGCAGC
ACAGCCTCCCACCCTGCCCGTGGAGGAGAAGAAGAAGATTCCAGATCCAGACAGCGATGACGTCTCTGAG
GTGGACGCGCGGCACATCATTGAGAATGCCAAGCAAGATGTCGATGATGAATATGGCGTGTCCCAGGCCC
TTGCACGTGGCCTGCAGTCCTACTATGCCGTGGCCCATGCTGTCACTGAGAGAGTGGACAAGCAGTCAGC
GCTTATGGTCAATGGTGTCCTCAAACAGTACCAGATCAAAGGTTTGGAGTGGCTGGTGTCCCTGTACAAC
AACAACCTGAACGGCATCCTGGCCGACGAGATGGGCCTGGGGAAGACCATCCAGACCATCGCGCTCATCA
CGTACCTCATGGAGCACAAACGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTTACAAGGGATCCCCAGCAGCA
AGACGGGCCTTTGTCCCCCAGCTCCGGAGTGGGAAGTTCAACGTCTTGCTGACGACGTACGAGTACATCA
TCAAAGACAAGCACATCCTCGCCAAGATCCGTTGGAAGTACATGATTGTGGACGAAGGTCACCGCATGAA
GAACCACCACTGCAAGCTGACGCAGGTGCTCAACACGCACTATGTGGCACCCCGCCGCCTGCTGCTGACG
GGCACACCGCTGCAGAACAAGCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACCGGGGAAAAGGTGGACCTGAA
TGAGGAGGAAACCATTCTCATCATCCGGCGTCTCCACAAAGTGCTGCGGCCCTTCTTGCTCCGACGACTC
AAGAAGGAAGTCGAGGCCCAGTTGCCCGAAAAGGTGGAGTACGTCATCAAGTGCGACATGTCTGCGCTGC
AGCGAGTGCTCTACCGCCACATGCAGGCCAAGGGCGTGCTGCTGACTGATGGCTCCGAGAAGGACAAGAA
GGGCAAAGGCGGCACCAAGACCCTGATGAACACCATCATGCAGCTGCGGAAGATCTGCAACCACCCCTAC
ATGTTCCAGCACATCGAGGAGTCCTTTTCCGAGCACTTGGGGTTCACTGGCGGCATTGTCCAAGGGCTGG
ACCTGTACCGAGCCTCGGGTAAATTTGAGCTTCTTGATAGAATTCTTCCCAAACTCCGAGCAACCAACCA
CAAAGTGCTGCTGTTCTGCCAAATGACCTCCCTCATGACCATCATGGAAGATTACTTTGCGTATCGCGGC
TTTAAATACCTCAGGCTTGATGGAACCACGAAGGCGGAGGACCGGGGCATGCTGCTGAAAACCTTCAACG
AGCCCGGCTCTGAGTACTTCATCTTCCTGCTCAGCACCCGGGCTGGGGGGCTCGGCCTGAACCTCCAGTC
GGCAGACACTGTGATCATTTTTGACAGCGACTGGAATCCTCACCAGGACCTGCAAGCGCAGGACCGAGCC
CACCGCATCGGGCAGCAGAACGAGGTGCGTGTGCTCCGCCTCTGCACCGTCAACAGCGTGGAGGAGAAGA
TCCTAGCTGCAGCCAAGTACAAGCTCAACGTGGACCAGAAGGTGATCCAGGCCGGCATGTTCGACCAGAA
GTCCTCCAGCCATGAGCGGCGCGCCTTCCTGCAGGCCATCCTGGAGCACGAGGAGCAGGATGAGGAGGAA
GACGAGGTGCCCGACGACGAGACCGTCAACCAGATGATCGCCCGGCACGAGGAGGAGTTTGATCTGTTCA
TGCGCATGGACCTGGACCGCAGGCGCGAGGAGGCCCGCAACCCCAAGCGGAAGCCGCGCCTCATGGAGGA
GGACGAGCTCCCCTCGTGGATCATCAAGGACGACGCGGAGGTGGAGCGGCTGACCTGTGAGGAGGAGGAG
GAGAAGATGTTCGGCCGTGGCTCCCGCCACCGCAAGGAGGTGGACTACAGCGACTCACTGACGGAGAAGC
AGTGGCTCAAGGCCATCGAGGAGGGCACGCTGGAGGAGATCGAAGAGGAGGTCCGGCAGAAGAAATCATC
ACGGAAGCGCAAGCGAGACAGCGACGCCGGCTCCTCCACCCCGACCACCAGCACCCGCAGCCGCGACAAG
GACGACGAGAGCAAGAAGCAGAAGAAGCGCGGGCGGCCGCCTGCCGAGAAACTCTCCCCTAACCCACCCA
ACCTCACCAAGAAGATGAAGAAGATTGTGGATGCCGTGATCAAGTACAAGGACAGCAGTGGACGTCAGCT
CAGCGAGGTCTTCATCCAGCTGCCCTCGCGAAAGGAGCTGCCCGAGTACTACGAGCTCATCCGCAAGCCC
GTGGACTTCAAGAAGATAAAGGAGCGCATTCGCAACCACAAGTACCGCAGCCTCAACGACCTAGAGAAGG
ACGTCATGCTCCTGTGCCAGAACGCACAGACCTTCAACCTGGAGGGCTCCCTGATCTATGAAGACTCCAT
CGTCTTGCAGTCGGTCTTCACCAGCGTGCGGCAGAAAATCGAGAAGGAGGATGACAGTGAAGGCGAGGAG
AGTGAGGAGGAGGAAGAGGGCGAGGAGGAAGGCTCCGAATCCGAATCTCGGTCCGTCAAAGTGAAGATCA
AGCTTGGCCGGAAGGAGAAGGCACAGGACCGGCTGAAGGGCGGCCGGCGGCGGCCGAGCCGAGGGTCCCG
AGCCAAGCCGGTCGTGAGTGACGATGACAGTGAGGAGGAACAAGAGGAGGACCGCTCAGGAAGTGGCAGC
GAAGAAGACTGAGCCCCGACATTCCAGTCTCGACCCCGAGCCCCTCGTTCCAGAGCTGAGATGGCATAGG
CCTTAGCAGTAACGGGTAGCAGCAGATGTAGTTTCAGACTTGGAGTAAAACTGTATAAACAAAAGAATCT
TCCATATTTATACAGCAGAGAAGCTGTAGGACTGTTTGTGACTGGCCCTGTCCTGGCATCAGTAGCATCT
GTAACAGCATTAACTGTCTTAAAGAGAGAGAGAGAGAATTCCGAATTGGGGAACACACGATACCTGTTTT
TCTTTTCCGTTGCTGGCAGTACTGTTGCGCCGCAGTTTGGAGTCACTGTAGTTAAGTGTGGATGCATGTG
CGTCACCGTCCACTCCTCCTACTGTATTTTATTGGACAGGTCAGACTCGCCGGGGGCCCGGCGAGGGTAT
GTCAGTGTCACTGGATGTCAAACAGTAATAAATTAAACCAACAACAAAACGCACAGCCAAAAAAAAA
Protein sequence of human transcription activator BRG1 isoform A
(GenBank Accession No. NP_001122321.1)
(SEQ ID NO: 15)
MSTPDPPLGGTPRPGPSPGPGPSPGAMLGPSPGPSPGSAHSMMGPSPGPPSAGHPIPTQGPGGYPQDNMH
QMHKPMESMHEKGMSDDPRYNQMKGMGMRSGGHAGMGPPPSPMDQHSQGYPSPLGGSEHASSPVPASGPS
SGPQMSSGPGGAPLDGADPQALGQQNRGPTPFNQNQLHQLRAQIMAYKMLARGQPLPDHLQMAVQGKRPM
PGMQQQMPTLPPPSVSATGPGPGPGPGPGPGPGPAPPNYSRPHGMGGPNMPPPGPSGVPPGMPGQPPGGP
PKPWPEGPMANAAAPTSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSILQHAKDFKEYH
RSVTGKIQKLTKAVATYHANTEREQKKENERIEKERMRRLMAEDEEGYRKLIDQKKDKRLAYLLQQTDEY
VANLTELVRQHKAAQVAKEKKKKKKKKKAENAEGQTPAIGPDGEPLDETSQMSDLPVKVIHVESGKILTG
TDAPKAGQLEAWLEMNPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQIKGLEWLVSLYN
NNLNGILADEMGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTHYVAPRRLLLT
GTPLQNKLPELWALLNFLLPTIFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLMTIMEDYFAYRG
FKYLRLDGTTKAEDRGMLLKTFNEPGSEYFIFLLSTRAGGLGLNLQSADTVIIFDSDWNPHQDLQAQDRA
HRIGQQNEVRVLRLCTVNSVEEKILAAAKYKLNVDQKVIQAGMFDQKSSSHERRAFLQAILEHEEQDESR
HCSTGSGSASFAHTAPPPAGVNPDLEEPPLKEEDEVPDDETVNQMIARHEEEFDLFMRMDLDRRREEARN
PKRKPRLMEEDELPSWIIKDDAEVERLTCEEEEEKMFGRGSRHRKEVDYSDSLTEKQWLKKITGKDIHDT
ASSVARGLQFQRGLQFCTRASKAIEEGTLEEIEEEVRQKKSSRKRKRDSDAGSSTPTTSTRSRDKDDESK
KQKKRGRPPAEKLSPNPPNLTKKMKKIVDAVIKYKDSSSGRQLSEVFIQLPSRKELPEYYELIRKPVDFK
KIKERIRNHKYRSLNDLEKDVMLLCQNAQTFNLEGSLIYEDSIVLQSVFTSVRQKIEKEDDSEGEESEEE
EEGEEEGSESESRSVKVKIKLGRKEKAQDRLKGGRRRPSRGSRAKPVVSDDDSEEEQEEDRSGSGSEED
Protein sequence of human transcription activator BRG1 isoform B
(GenBank Accession No. NP_001122316.1)
(SEQ ID NO: 16)
MSTPDPPLGGTPRPGPSPGPGPSPGAMLGPSPGPSPGSAHSMMGPSPGPPSAGHPIPTQGPGGYPQDNMH
QMHKPMESMHEKGMSDDPRYNQMKGMGMRSGGHAGMGPPPSPMDQHSQGYPSPLGGSEHASSPVPASGPS
SGPQMSSGPGGAPLDGADPQALGQQNRGPTPFNQNQLHQLRAQIMAYKMLARGQPLPDHLQMAVQGKRPM
PGMQQQMPTLPPPSVSATGPGPGPGPGPGPGPGPAPPNYSRPHGMGGPNMPPPGPSGVPPGMPGQPPGGP
PKPWPEGPMANAAAPTSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSILQHAKDFKEYH
RSVTGKIQKLTKAVATYHANTEREQKKENERIEKERMRRLMAEDEEGYRKLIDQKKDKRLAYLLQQTDEY
VANLTELVRQHKAAQVAKEKKKKKKKKKAENAEGQTPAIGPDGEPLDETSQMSDLPVKVIHVESGKILTG
TDAPKAGQLEAWLEMNPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQIKGLEWLVSLYN
NNLNGILADEMGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTHYVAPRRLLLT
GTPLQNKLPELWALLNFLLPTIFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLMTIMEDYFAYRG
FKYLRLDGTTKAEDRGMLLKTFNEPGSEYFIFLLSTRAGGLGLNLQSADTVIIFDSDWNPHQDLQAQDRA
HRIGQQNEVRVLRLCTVNSVEEKILAAAKYKLNVDQKVIQAGMFDQKSSSHERRAFLQAILEHEEQDESR
HCSTGSGSASFAHTAPPPAGVNPDLEEPPLKEEDEVPDDETVNQMIARHEEEFDLFMRMDLDRRREEARN
PKRKPRLMEEDELPSWIIKDDAEVERLTCEEEEEKMFGRGSRHRKEVDYSDSLTEKQWLKAIEEGTLEEI
EEEVRQKKSSRKRKRDSDAGSSTPTTSTRSRDKDDESKKQKKRGRPPAEKLSPNPPNLTKKMKKIVDAVI
KYKDSSSGRQLSEVFIQLPSRKELPEYYELIRKPVDFKKIKERIRNHKYRSLNDLEKDVMLLCQNAQTFN
LEGSLIYEDSIVLQSVFTSVRQKIEKEDDSEGEESEEEEEGEEEGSESESRSVKVKIKLGRKEKAQDRLK
GGRRRPSRGSRAKPVVSDDDSEEEQEEDRSGSGSEED
Protein sequence of human transcription activator BRG1 isoform C
(GenBank Accession No. NP_001122317.1)
(SEQ ID NO: 17)
MSTPDPPLGGTPRPGPSPGPGPSPGAMLGPSPGPSPGSAHSMMGPSPGPPSAGHPIPTQGPGGYPQDNMH
QMHKPMESMHEKGMSDDPRYNQMKGMGMRSGGHAGMGPPPSPMDQHSQGYPSPLGGSEHASSPVPASGPS
SGPQMSSGPGGAPLDGADPQALGQQNRGPTPFNQNQLHQLRAQIMAYKMLARGQPLPDHLQMAVQGKRPM
PGMQQQMPTLPPPSVSATGPGPGPGPGPGPGPGPAPPNYSRPHGMGGPNMPPPGPSGVPPGMPGQPPGGP
PKPWPEGPMANAAAPTSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSILQHAKDFKEYH
RSVTGKIQKLTKAVATYHANTEREQKKENERIEKERMRRLMAEDEEGYRKLIDQKKDKRLAYLLQQTDEY
VANLTELVRQHKAAQVAKEKKKKKKKKKAENAEGQTPAIGPDGEPLDETSQMSDLPVKVIHVESGKILTG
TDAPKAGQLEAWLEMNPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQIKGLEWLVSLYN
NNLNGILADEMGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTHYVAPRRLLLT
GTPLQNKLPELWALLNFLLPTIFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLMTIMEDYFAYRG
FKYLRLDGTTKAEDRGMLLKTFNEPGSEYFIFLLSTRAGGLGLNLQSADTVIIFDSDWNPHQDLQAQDRA
HRIGQQNEVRVLRLCTVNSVEEKILAAAKYKLNVDQKVIQAGMFDQKSSSHERRAFLQAILEHEEQDEEE
DEVPDDETVNQMIARHEEEFDLFMRMDLDRRREEARNPKRKPRLMEEDELPSWIIKDDAEVERLTCEEEE
EKMFGRGSRHRKEVDYSDSLTEKQWLKTLKAIEEGTLEEIEEEVRQKKSSRKRKRDSDAGSSTPTTSTRS
RDKDDESKKQKKRGRPPAEKLSPNPPNLTKKMKKIVDAVIKYKDSSSGRQLSEVFIQLPSRKELPEYYEL
IRKPVDFKKIKERIRNHKYRSLNDLEKDVMLLCQNAQTFNLEGSLIYEDSIVLQSVFTSVRQKIEKEDDS
EGEESEEEEEGEEEGSESESRSVKVKIKLGRKEKAQDRLKGGRRRPSRGSRAKPVVSDDDSEEEQEEDRS
GSGSEED
Protein sequence of human transcription activator BRG1 isoform D
(GenBank Accession No. NP_001122318.1)
(SEQ ID NO: 18)
MSTPDPPLGGTPRPGPSPGPGPSPGAMLGPSPGPSPGSAHSMMGPSPGPPSAGHPIPTQGPGGYPQDNMH
QMHKPMESMHEKGMSDDPRYNQMKGMGMRSGGHAGMGPPPSPMDQHSQGYPSPLGGSEHASSPVPASGPS
SGPQMSSGPGGAPLDGADPQALGQQNRGPTPFNQNQLHQLRAQIMAYKMLARGQPLPDHLQMAVQGKRPM
PGMQQQMPTLPPPSVSATGPGPGPGPGPGPGPGPAPPNYSRPHGMGGPNMPPPGPSGVPPGMPGQPPGGP
PKPWPEGPMANAAAPTSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSILQHAKDFKEYH
RSVTGKIQKLTKAVATYHANTEREQKKENERIEKERMRRLMAEDEEGYRKLIDQKKDKRLAYLLQQTDEY
VANLTELVRQHKAAQVAKEKKKKKKKKKAENAEGQTPAIGPDGEPLDETSQMSDLPVKVIHVESGKILTG
TDAPKAGQLEAWLEMNPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQIKGLEWLVSLYN
NNLNGILADEMGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTHYVAPRRLLLT
GTPLQNKLPELWALLNFLLPTIFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLMTIMEDYFAYRG
FKYLRLDGTTKAEDRGMLLKTFNEPGSEYFIFLLSTRAGGLGLNLQSADTVIIFDSDWNPHQDLQAQDRA
HRIGQQNEVRVLRLCTVNSVEEKILAAAKYKLNVDQKVIQAGMFDQKSSSHERRAFLQAILEHEEQDEEE
DEVPDDETVNQMIARHEEEFDLFMRMDLDRRREEARNPKRKPRLMEEDELPSWIIKDDAEVERLTCEEEE
EKMFGRGSRHRKEVDYSDSLTEKQWLKTLKAIEEGTLEEIEEEVRQKKSSRKRKRDSDAGSSTPTTSTRS
RDKDDESKKQKKRGRPPAEKLSPNPPNLTKKMKKIVDAVIKYKDSSGRQLSEVFIQLPSRKELPEYYELI
RKPVDFKKIKERIRNHKYRSLNDLEKDVMLLCQNAQTFNLEGSLIYEDSIVLQSVFTSVRQKIEKEDDSE
GEESEEEEEGEEEGSESESRSVKVKIKLGRKEKAQDRLKGGRRRPSRGSRAKPVVSDDDSEEEQEEDRSG
SGSEED
Protein sequence of human transcription activator BRG1 isoform E
(GenBank Accession No. NP_001122319.1)
(SEQ ID NO: 19)
MSTPDPPLGGTPRPGPSPGPGPSPGAMLGPSPGPSPGSAHSMMGPSPGPPSAGHPIPTQGPGGYPQDNMH
QMHKPMESMHEKGMSDDPRYNQMKGMGMRSGGHAGMGPPPSPMDQHSQGYPSPLGGSEHASSPVPASGPS
SGPQMSSGPGGAPLDGADPQALGQQNRGPTPFNQNQLHQLRAQIMAYKMLARGQPLPDHLQMAVQGKRPM
PGMQQQMPTLPPPSVSATGPGPGPGPGPGPGPGPAPPNYSRPHGMGGPNMPPPGPSGVPPGMPGQPPGGP
PKPWPEGPMANAAAPTSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSILQHAKDFKEYH
RSVTGKIQKLTKAVATYHANTEREQKKENERIEKERMRRLMAEDEEGYRKLIDQKKDKRLAYLLQQTDEY
VANLTELVRQHKAAQVAKEKKKKKKKKKAENAEGQTPAIGPDGEPLDETSQMSDLPVKVIHVESGKILTG
TDAPKAGQLEAWLEMNPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQIKGLEWLVSLYN
NNLNGILADEMGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTHYVAPRRLLLT
GTPLQNKLPELWALLNFLLPTIFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLMTIMEDYFAYRG
FKYLRLDGTTKAEDRGMLLKTFNEPGSEYFIFLLSTRAGGLGLNLQSADTVIIFDSDWNPHQDLQAQDRA
HRIGQQNEVRVLRLCTVNSVEEKILAAAKYKLNVDQKVIQAGMFDQKSSSHERRAFLQAILEHEEQDEEE
DEVPDDETVNQMIARHEEEFDLFMRMDLDRRREEARNPKRKPRLMEEDELPSWIIKDDAEVERLTCEEEE
EKMFGRGSRHRKEVDYSDSLTEKQWLKAIEEGTLEEIEEEVRQKKSSRKRKRDSDAGSSTPTTSTRSRDK
DDESKKQKKRGRPPAEKLSPNPPNLTKKMKKIVDAVIKYKDSSSGRQLSEVFIQLPSRKELPEYYELIRK
PVDFKKIKERIRNHKYRSLNDLEKDVMLLCQNAQTFNLEGSLIYEDSIVLQSVFTSVRQKIEKEDDSEGE
ESEEEEEGEEEGSESESRSVKVKIKLGRKEKAQDRLKGGRRRPSRGSRAKPVVSDDDSEEEQEEDRSGSG
SEED
Protein sequence of human transcription activator BRG1 isoform F
(GenBank Accession No. NP_001122320.1
(SEQ ID NO: 20)
MSTPDPPLGGTPRPGPSPGPGPSPGAMLGPSPGPSPGSAHSMMGPSPGPPSAGHPIPTQGPGGYPQDNMH
QMHKPMESMHEKGMSDDPRYNQMKGMGMRSGGHAGMGPPPSPMDQHSQGYPSPLGGSEHASSPVPASGPS
SGPQMSSGPGGAPLDGADPQALGQQNRGPTPFNQNQLHQLRAQIMAYKMLARGQPLPDHLQMAVQGKRPM
PGMQQQMPTLPPPSVSATGPGPGPGPGPGPGPGPAPPNYSRPHGMGGPNMPPPGPSGVPPGMPGQPPGGP
PKPWPEGPMANAAAPTSTPQKLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSILQHAKDFKEYH
RSVTGKIQKLTKAVATYHANTEREQKKENERIEKERMRRLMAEDEEGYRKLIDQKKDKRLAYLLQQTDEY
VANLTELVRQHKAAQVAKEKKKKKKKKKAENAEGQTPAIGPDGEPLDETSQMSDLPVKVIHVESGKILTG
TDAPKAGQLEAWLEMNPGYEVAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMVNGVLKQYQIKGLEWLVSLYN
NNLNGILADEMGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTHYVAPRRLLLT
GTPLQNKLPELWALLNFLLPTIFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVLLFCQMTSLMTIMEDYFAYRG
FKYLRLDGTTKAEDRGMLLKTFNEPGSEYFIFLLSTRAGGLGLNLQSADTVIIFDSDWNPHQDLQAQDRA
HRIGQQNEVRVLRLCTVNSVEEKILAAAKYKLNVDQKVIQAGMFDQKSSSHERRAFLQAILEHEEQDEEE
DEVPDDETVNQMIARHEEEFDLFMRMDLDRRREEARNPKRKPRLMEEDELPSWIIKDDAEVERLTCEEEE
EKMFGRGSRHRKEVDYSDSLTEKQWLKAIEEGTLEEIEEEVRQKKSSRKRKRDSDAGSSTPTTSTRSRDK
DDESKKQKKRGRPPAEKLSPNPPNLTKKMKKIVDAVIKYKDSSGRQLSEVFIQLPSRKELPEYYELIRKP
VDFKKIKERIRNHKYRSLNDLEKDVMLLCQNAQTFNLEGSLIYEDSIVLQSVFTSVRQKIEKEDDSEGEE
SEEEEEGEEEGSESESRSVKVKIKLGRKEKAQDRLKGGRRRPSRGSRAKPVVSDDDSEEEQEEDRSGSGS
EED

### SMARCA2 Antagonists

SMARCA2 antagonists are known in the art, and include, for example, the compounds shown in Table 2 below:

TABLE 2
SMARCA2 inhibitors

| | | | |
|---|---|---|---|
| BMCL 2968 | I-BET151 | JQ1 | PFI3 |
| 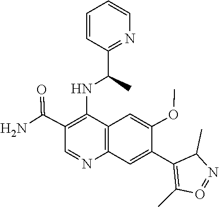 | 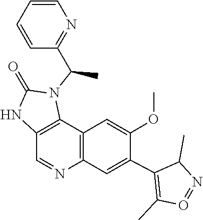 | 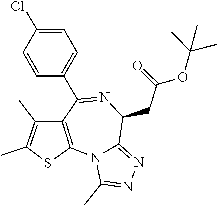 | 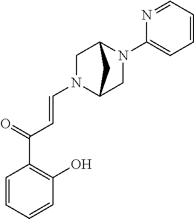 |

Additional SMARCA2 inhibitors are known in the art, or will be apparent to the person of ordinary skill in the art based on the present disclosure. The disclosure is not limited in this respect.

In certain aspects of the disclosure an antagonist or inhibitor of SMARCA2 “selectively inhibits” or “selectively antagonizes” SMARCA2 activity of a cell when it inhibits SMARCA2 activity more effectively than it inhibits SMARCA4 activity. For example, in some embodiments the selective inhibitor or antagonist has an IC50 for SMARCA2 that is at least 40 percent lower than the IC50 for SMARCA4. In some embodiments, the selective inhibitor or antagonist has an IC50 for the SMARCA2 that is at least 50 percent lower than the IC50 for SMARCA4. In some embodiments, the selective inhibitor or antagonist has an IC50 for the SMARCA2 that is at least 60 percent lower than the IC50 for SMARCA4. In some embodiments, the selective inhibitor or antagonist has an IC50 for SMARCA2 that is at least 70 percent lower than the IC50 for SMARCA4. In some embodiments, the selective inhibitor or antagonist has an IC50 for SMARCA2 that is at least 80 percent lower than the IC50 for SMARCA4. In some embodiments, the selective inhibitor or antagonist has an IC50 for SMARCA2 that is at least 90 percent lower than the IC50 for SMARCA4. In some embodiments, the selective antagonist or inhibitor of SMARCA2 exerts essentially no inhibitory effect on SMARCA4.

In some embodiments, a SMARCA2 antagonist (e.g., a SMARCA2 inhibitor) inhibits SMARCA2 activity at least 2-fold more efficiently than SMARCA4 activity. In some embodiments, a SMARCA2 antagonist (e.g., a SMARCA2 inhibitor) inhibits SMARCA2 activity at least 5-fold more efficiently than SMARCA4 activity. In some embodiments, a SMARCA2 antagonist (e.g., a SMARCA2 inhibitor) inhibits SMARCA2 activity at least 10-fold more efficiently than SMARCA4 activity. In some embodiments, a SMARCA2 antagonist (e.g., a SMARCA2 inhibitor) inhibits SMARCA2 activity at least 20-fold fold more efficiently than SMARCA4 activity. In some embodiments, a SMARCA2 antagonist (e.g., a SMARCA2 inhibitor) inhibits SMARCA2 activity at least 50-fold more efficiently than SMARCA4 activity. In some embodiments, a SMARCA2 antagonist (e.g., a SMARCA2 inhibitor) inhibits SMARCA2 activity at least 100-fold more efficiently than SMARCA4 activity. In some embodiments, a SMARCA2 antagonist (e.g., a SMARCA2 inhibitor) inhibits SMARCA2 activity at least 1000-fold more efficiently than SMARCA4 activity. In some embodiments, a SMARCA2 antagonist (e.g., a SMARCA2 inhibitor) inhibits SMARCA2 activity at least 10000-fold more efficiently than SMARCA4 activity. In some embodiments, a SMARCA2 antagonist (e.g., a SMARCA2 inhibitor) inhibits SMARCA2 activity at least 100000-fold more efficiently than SMARCA4 activity.

In some embodiments, reduced expression or function, or loss of function, of SMARCA4 confers sensitivity of said cell to inhibition of SMARCA2.

In certain aspects of the disclosure, the inhibitor or antagonist targets the helicase domain of SMARCA2. In some embodiments, the inhibitor or antagonist targets the ATP domain of SMARCA2. In some embodiments, the inhibitor or antagonist does not target the bromodomain of SMARCA2. In some embodiments, the inhibitor or antagonist targets the bromodomain of SMARCA2.

In some aspects, a SMARCA2 antagonist (e.g., a SMARCA2 inhibitor) inhibits SMARCA2 helicase activity. In some embodiments, a SMARCA2 antagonist (e.g., a SMARCA2 inhibitor) inhibits SMARCA2 helicase activity by at least 10%. In some embodiments, a SMARCA2 antagonist (e.g., a SMARCA2 inhibitor) inhibits SMARCA2 helicase activity by at least 20%. In some embodiments, a SMARCA2 antagonist (e.g., a SMARCA2 inhibitor) inhibits SMARCA2 helicase activity by at least 30%. In some embodiments, a SMARCA2 antagonist (e.g., a SMARCA2 inhibitor) inhibits SMARCA2 helicase activity by at least 40%. In some embodiments, a SMARCA2 antagonist (e.g., a SMARCA2 inhibitor) inhibits SMARCA2 helicase activity by at least 50%. In some embodiments, a SMARCA2 antagonist (e.g., a SMARCA2 inhibitor) inhibits SMARCA2 helicase activity by at least 60%. In some embodiments, a SMARCA2 antagonist (e.g., a SMARCA2 inhibitor) inhibits SMARCA2 helicase activity by at least 70%. In some embodiments, a SMARCA2 antagonist (e.g., a SMARCA2 inhibitor) inhibits SMARCA2 helicase activity by at least 80%. In some embodiments, a SMARCA2 antagonist (e.g., a SMARCA2 inhibitor) inhibits SMARCA2 helicase activity by at least 90%. In some embodiments, a SMARCA2 antagonist (e.g., a SMARCA2 inhibitor) inhibits SMARCA2 helicase activity by at least 95%. In some embodiments, a SMARCA2 antagonist (e.g., a SMARCA2 inhibitor) inhibits SMARCA2 helicase activity by at least 98%. In some embodiments, a SMARCA2 antagonist (e.g., a SMARCA2 inhibitor) inhibits SMARCA2 helicase activity by or at least 99%. In some embodiments, a SMARCA2 antagonist (e.g., a SMARCA2 inhibitor) inhibits SMARCA2 helicase activity and abolishes SMARCA2 activity.

In some aspects, a SMARCA2 antagonist (e.g., a SMARCA2 inhibitor) inhibits SMARCA2 ATPase activity. In some embodiments, a SMARCA2 antagonist (e.g., a SMARCA2 inhibitor) inhibits SMARCA2 ATPase activity by at least 10%. In some embodiments, a SMARCA2 antagonist (e.g., a SMARCA2 inhibitor) inhibits SMARCA2 ATPase activity by at least 20%. In some embodiments, a SMARCA2 antagonist (e.g., a SMARCA2 inhibitor) inhibits SMARCA2 ATPase activity by at least 30%. In some embodiments, a SMARCA2 antagonist (e.g., a SMARCA2 inhibitor) inhibits SMARCA2 ATPase activity by at least 40%. In some embodiments, a SMARCA2 antagonist (e.g., a SMARCA2 inhibitor) inhibits SMARCA2 ATPase activity by at least 50%. In some embodiments, a SMARCA2 antagonist (e.g., a SMARCA2 inhibitor) inhibits SMARCA2 ATPase activity by at least 60%. In some embodiments, a SMARCA2 antagonist (e.g., a SMARCA2 inhibitor) inhibits SMARCA2 ATPase activity by at least 70%. In some embodiments, a SMARCA2 antagonist (e.g., a SMARCA2 inhibitor) inhibits SMARCA2 ATPase activity by at least 80%. In some embodiments, a SMARCA2 antagonist (e.g., a SMARCA2 inhibitor) inhibits SMARCA2 ATPase activity by at least 90%. In some embodiments, a SMARCA2 antagonist (e.g., a SMARCA2 inhibitor) inhibits SMARCA2 ATPase activity by at least 95%. In some embodiments, a SMARCA2 antagonist (e.g., a SMARCA2 inhibitor) inhibits SMARCA2 ATPase activity by at least 98%. In some embodiments, a SMARCA2 antagonist (e.g., a SMARCA2 inhibitor) inhibits SMARCA2 ATPase activity by or at least 99%. In some embodiments, a SMARCA2 antagonist (e.g., a SMARCA2 inhibitor) inhibits SMARCA2 ATPase activity and abolishes SMARCA2 activity

In certain aspects of the disclosure, the SMARCA2 antagonist or inhibitor inhibits SMARCA2 activity. Inhibition of SMARCA2 activity can be detected using any suitable method. The inhibition can be measured, for example, either in terms of rate of SMARCA2 activity or as product of SMARCA2 activity.

The inhibition is a measurable inhibition compared to a suitable control. In some embodiments, inhibition is at least 10 percent inhibition compared to a suitable control. That is, the rate of enzymatic activity or the amount of product with the inhibitor is less than or equal to 90 percent of the corresponding rate or amount made without the inhibitor. In some embodiments, inhibition is at least 20, 25, 30, 40, 50, 60, 70, 75, 80, 90, or 95 percent inhibition compared to a suitable control. In some embodiments, inhibition is at least 99 percent inhibition compared to a suitable control. That is, the rate of enzymatic activity or the amount of product with the inhibitor is less than or equal to 1 percent of the corresponding rate or amount made without the inhibitor.

### Pharmaceutical Formulations

The disclosure also provides pharmaceutical compositions comprising a compound of the disclosure or pharmaceutically acceptable salts thereof, and one or more other therapeutic agents disclosed herein, mixed with pharmaceutically suitable carriers or excipient(s) at doses to treat or prevent a disease or condition as described herein. The pharmaceutical compositions of the disclosure can also be administered in combination with other therapeutic agents or therapeutic modalities simultaneously, sequentially, or in alternation.

Mixtures of compositions of the disclosure can also be administered to the patient as a simple mixture or in suitable formulated pharmaceutical compositions. For example, some aspects of the disclosure relate to a pharmaceutical composition comprising a therapeutically effective dose of a compound of the disclosure, or a pharmaceutically acceptable salt, hydrate, enantiomer or stereoisomer thereof; one or more other therapeutic agents, and a pharmaceutically acceptable diluent or carrier.

A “pharmaceutical composition” is a formulation containing the compounds of the disclosure in a form suitable for administration to a subject. A compound of the disclosure and one or more other therapeutic agents described herein each can be formulated individually or in multiple pharmaceutical compositions in any combinations of the active ingredients. Accordingly, one or more administration routes can be properly elected based on the dosage form of each pharmaceutical composition. Alternatively, a compound of the disclosure and one or more other therapeutic agents described herein can be formulated as one pharmaceutical composition.

In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In some aspects, the disease or condition to be treated is cancer. In some aspects, the disease or condition to be treated is a cell proliferative disorder.

In certain embodiments the therapeutically effective amount of each pharmaceutical agent used in combination will be lower when used in combination in comparison to monotherapy with each agent alone. Such lower therapeutically effective amount could afford for lower toxicity of the therapeutic regimen.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the SMARCA2 antagonists (e.g., inhibitors) described herein, other therapeutic agents described herein, compositions comprising a compound of the disclosure and one or more other therapeutic agents, or the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In some aspects, dosages can range from about 1 mg/kg per day to about 1000 mg/kg per day. In some aspects, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The composition of the disclosure is capable of further forming salts. The composition of the disclosure is capable of forming more than one salt per molecule, e.g., mono-, di-, tri-. All of these forms are also contemplated within the scope of the claimed invention.

As used herein, “pharmaceutically acceptable salts” refer to derivatives of the compounds of the disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

The composition of the disclosure may also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The composition, or pharmaceutically acceptable salts or solvates thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the disclosure are apparent from the different examples. The provided examples illustrate different components and methodology useful in practicing the disclosure. The examples do not limit the claimed invention. Based on the present disclosure the skilled artisan can identify and employ other components and methodology useful for practicing the disclosure.

As used herein, a “subject in need thereof” is a subject having a disorder associated with a decreased level of activity or function of SMARCA4 compared to a control level, or a subject having an increased risk of developing such disorder relative to the population at large. Preferably, a subject in need thereof has cancer. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

In some embodiments, the control level is a level of SMARCA4 expression in a subject or cell from a subject that does not have cancer. In some embodiments, the control level may be a level of SMARCA4 expression in a subject or cell from a subject belonging to a certain population, wherein the level is equal or about equal to the average level of expression or function of SMARCA4 observed in said population. In some embodiments, the control level may be a level of expression or function of SMARCA4 that is equal or about equal to the average level of expression or function of SMARCA4 in the population at large.

The subject of the disclosure includes any human subject who has been diagnosed with, has symptoms of, or is at risk of developing a cancer or a precancerous condition. The subject of the disclosure includes any human subject expressing a mutant SMARCA4 gene. For example, a mutant SMARCA4 comprises one or more mutations, wherein the mutation is a substitution, a point mutation, a nonsense mutation, a missense mutation, a deletion, or an insertion or any other SMARCA4 mutation described herein or otherwise known in the art to be associated with a loss of function of SMARCA4.

A subject in need thereof may have refractory or resistant cancer. “Refractory or resistant cancer” means cancer that does not respond to an established line of treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

In some embodiments, a subject in need thereof may have a secondary cancer as a result of a previous therapy. “Secondary cancer” means cancer that arises due to or as a result from previous carcinogenic therapies, such as chemotherapy.

The subject may also exhibit decreased function or expression of SMARCA4, or loss of function of SMARCA4.

In some embodiments, the subject is a participant in a clinical trial. In some embodiments, a criterion for participation of a subject in the clinical trial is a decreased activity or function of SMARCA4, or loss of function of SMARCA4, in said subject or a cell of said subject.

As used herein, the term “responsiveness” is interchangeable with terms “responsive”, “sensitive”, and “sensitivity”, and it is meant that a subject is showing therapeutic responses when administered a composition of the disclosure, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered a composition of the disclosure, e.g., tumor cells or tumor tissues of the subject undergo apoptosis and/or necrosis, and/or display reduced growing, dividing, or proliferation.

As used herein, “sample” means any biological sample derived from the subject, includes but is not limited to, cells, tissues samples, body fluids (including, but not limited to, mucus, blood, plasma, serum, urine, saliva, and semen), tumor cells, and tumor tissues. Preferably, the sample is selected from bone marrow, peripheral blood cells, blood, plasma and serum. Samples can be provided by the subject under treatment or testing. Alternatively samples can be obtained by the physician according to routine practice in the art.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the disclosure, or a pharmaceutically acceptable salt or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the disclosure, or a pharmaceutically acceptable salt or solvate thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

As used herein, “treating” or “treat” describes the management and care of a patient for the purpose of combating a disease, condition, or disorder and includes the administration of a compound of the disclosure, or a pharmaceutically acceptable salt or solvate thereof, to alleviate the symptoms or complications of a disease, condition or disorder, or to eliminate the disease, condition or disorder.

A composition of the disclosure, or a pharmaceutically acceptable salt or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In some embodiments, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In some aspects of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

### Cancer

A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, compositions of the disclosure may be used to treat a cancer selected from the group consisting of a hematologic cancer of the disclosure or a hematologic cell proliferative disorder of the disclosure. A hematologic cancer of the disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions of the disclosure may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructural heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, compositions of the disclosure may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon, and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, malignant growths or lesions of the prostate and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the disclosure may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Preferably, compositions of the disclosure may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphocytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructural heterogeneity (e.g., mixed cell types).

Preferably, compound of the disclosure, or a pharmaceutically acceptable salt or solvate thereof, may be used to treat breast cancer. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the disclosure, or a pharmaceutically acceptable salt or solvate thereof, may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pNO, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

In some embodiments, a cancer that is to be treated is a cancer in which a member of the SWI/SNF complex, e.g., SMARCA4, is mutated or exhibits a loss of function (e.g., a decrease of enzymatic activity). For example, a cancer to be treated may be a cancer in which SMARCA4 is mutated. Non limiting examples of cancers in which SMARCA4 mutations occur include small cell carcinoma of the ovary of the hypercalcemic type (SCCOHT), bladder cancer, stomach cancer, lung cancer (e.g., non-small cell lung cancer), glioblastoma brain tumors (glioma, GBM), head and neck cancer, kidney cancer, uterine cancer, cervical cancer, and pancreatic cancer.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer can result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the disclosure, or a pharmaceutically acceptable salt, solvate, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the disclosure, or a pharmaceutically acceptable salt, solvate, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the disclosure, or a pharmaceutically acceptable salt or solvate thereof, acts selectively on a cancer or precancerous cell but not on a normal cell. Preferably, a compound of the disclosure, or a pharmaceutically acceptable salt or solvate thereof, acts selectively to modulate one molecular target (e.g., a target helicase, such as SMARCA2) but does not significantly modulate another molecular target (e.g., a different helicase, such as SMARCA4, or a non-helicase enzyme, e.g., in the case of a SMARCA2 ATPase inhibitor, the ATPase activity of a different helicase, or a different protein having ATPase activity). The disclosure also provides a method for selectively inhibiting the activity of an enzyme, such as a helicase (e.g., SMARCA2). Preferably, a SMARCA2 inhibitor selectively inhibits SMARCA2, e.g., a helicase or ATPase activity of SMARCA2, relative to inhibiting a second, different enzyme, e.g., a different helicase (e.g., SMARCA4) or a different enzyme exhibiting ATPase activity, if the inhibition of SMARCA2 is greater than two times the inhibition of the second, different enzyme. In some embodiments, selective SMARCA2 inhibition occurs if the inhibition of SMARCA2 is greater than five times, greater than 10 times, greater than fifty times, greater than 100 times, or greater than 1000 times the inhibition of the second, different enzyme. For example, in some embodiments, SMARCA2 inhibition would be said to occur selectively over SMARCA4 inhibition if SMARCA2 helicase activity inhibition is greater than 2-fold the SMARCA4 inhibition.

A composition of the disclosure, e.g., a composition comprising SMARCA2 inhibitor, and one or more other therapeutic agents, such as prednisone, can modulate the activity of a molecular target (e.g., a target helicase). Modulating refers to stimulating or inhibiting an activity of a molecular target. Preferably, a compound of the disclosure, or a pharmaceutically acceptable salt or solvate thereof, modulates the activity of a molecular target if it stimulates or inhibits the activity of the molecular target by at least 2-fold relative to the activity of the molecular target under the same conditions but lacking only the presence of said compound. More preferably, a compound of the disclosure, or a pharmaceutically acceptable salt or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A composition of the disclosure, e.g., a composition comprising SMARCA2 inhibitor, and one or more other therapeutic agents, such as prednisone, can modulate the activity of a molecular target (e.g., a target helicase). Modulating refers to stimulating or inhibiting an activity of a molecular target. Preferably, a compound of the disclosure, or a pharmaceutically acceptable salt or solvate thereof, modulates the activity of a molecular target if it stimulates or inhibits the activity of the molecular target by at least 2-fold relative to the activity of the molecular target under the same conditions but lacking only the presence of said compound. More preferably, a compound of the disclosure, or a pharmaceutically acceptable salt or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A composition of the disclosure does not significantly modulate the activity of a molecular target if the addition of the compound does not stimulate or inhibit the activity of the molecular target by greater than 10% relative to the activity of the molecular target under the same conditions but lacking only the presence of said compound.

Administering a composition of the disclosure to a cell or a subject in need thereof can result in modulation (i.e., stimulation or inhibition) of an activity of a helicase of interest.

Administering a compound of the disclosure, e.g., a composition comprising a SMARCA2 inhibitor, and one or more other therapeutic agents, such as prednisone, to a cell or a subject in need thereof results in modulation (i.e., stimulation or inhibition) of an activity of an intracellular target (e.g., substrate). Several intracellular targets can be modulated with the compounds of the disclosure, including, but not limited to, helicases.

Activating refers to placing a composition of matter (e.g., protein or nucleic acid) in a state suitable for carrying out a desired biological function. A composition of matter capable of being activated also has an unactivated state. An activated composition of matter may have an inhibitory or stimulatory biological function, or both.

Elevation refers to an increase in a desired biological activity of a composition of matter (e.g., a protein or a nucleic acid). Elevation may occur through an increase in concentration of a composition of matter.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In some aspects, cell death occurs by apoptosis.

Preferably, an effective amount of a composition of the disclosure, or a pharmaceutically acceptable salt or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In some aspects, cell death occurs by apoptosis.

Contacting a cell with a composition of the disclosure, or a pharmaceutically acceptable salt or solvate thereof, can induce or activate cell death selectively in cancer cells. Administering to a subject in need thereof a compound of the disclosure, or a pharmaceutically acceptable salt or solvate thereof, can induce or activate cell death selectively in cancer cells. Contacting a cell with a composition of the disclosure, or a pharmaceutically acceptable salt or solvate thereof, can induce cell death selectively in one or more cells affected by a cell proliferative disorder. Preferably, administering to a subject in need thereof a composition of the disclosure, or a pharmaceutically acceptable salt or solvate thereof, induces cell death selectively in one or more cells affected by a cell proliferative disorder.

The disclosure relates to a method of treating or preventing cancer by administering a composition of the disclosure, or a pharmaceutically acceptable salt or solvate thereof, to a subject in need thereof, where administration of the composition of the disclosure, or a pharmaceutically acceptable salt or solvate thereof, results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

### Example 1

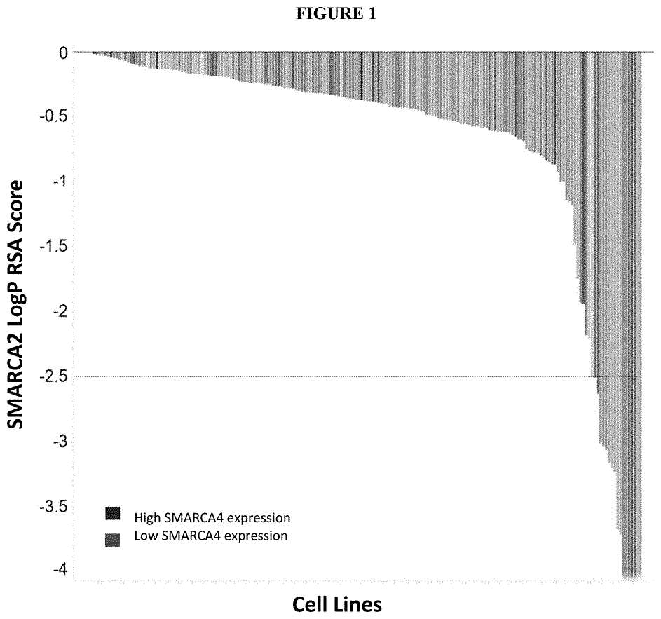

Sensitivity to knockout of SMARCA2 through CRISPR/Cas9-mediated gene knockout was determined by CRISPR/Cas9 pooled screening. A large population of cells was infected with a pooled library of barcoded sgRNA guides to genes of interest. For proliferation-based screens, the barcode/CRISPR representation was measured at the start and end of the experiment by sequencing of genomic DNA, and the relative enrichment/decrease in CRISPR sgRNAs identified genes for which knockout altered proliferation rate. A custom CRISPR lentiviral library with 6500 small guide RNAs targeting over 600 epigenetic genes was generated, and screened against 195 cell lines over a time course of up to 40 days. KRas was included as a positive control in the CRISPR/Cas9 library, and it was observed that sensitivity to KRas knockout was highly correlated with KRas mutations. SMARCA4 null or mutant cells, including A549 and NCIH1299 lung cancer cell lines, were found to be sensitive to SMARCA2 knockout (FIG. 1). The synthetic lethal relationship between SMARCA2 and SMARCA4 was further validated in the literature (Hoffman et al. PNAS, 2013, 111(8), 3128-3133; Wilson et al, Mol. Cell Biol., 2014, 34(6),1136-44; Vangamundi et al. Cancer Res. 2015, 75(18):3865-78; and Oike et al., Cancer Res. 2013 Sep. 1; 73(17), 5508-18; the contents of each of which are incorporated herein by reference in their entireties)

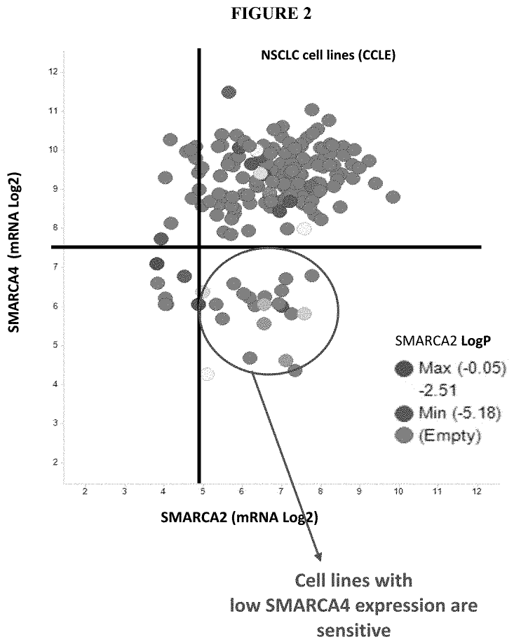

To further investigate the relationship between SMARCA4 expression and sensitivity to SMARCA2 inhibition, a panel of non-small cell lung cancer (NSCLC) lines was tested for protein levels of SMARCA2 and SMARCA4. A transcriptomic analysis of NSCLC cell lines that have RNA seq. data available in Cancer Cell Line Encyclopedia (CCLE) is shown in FIG. 2. Cell lines with low SMARCA4 expression were found to be sensitive to SMARCA2 inhibition, suggesting that loss of SMARCA4 predicts response to SMARCA2 inhibition and is a potential patient stratification biomarker. It is also suggested that SMARCA4 mutations under-predict protein loss, and that hence, an understanding of protein levels of SMARCA2 and SMARCA4 expression is critical, calling for better analysis of this patient population, e.g. via immunohistochemistry assays or multiplex protein assays to assess protein expression in clinical samples.

### Example 2: SMARCA2/4 Immunohistochemistry Assay to Assess Protein Expression in Clinical Samples

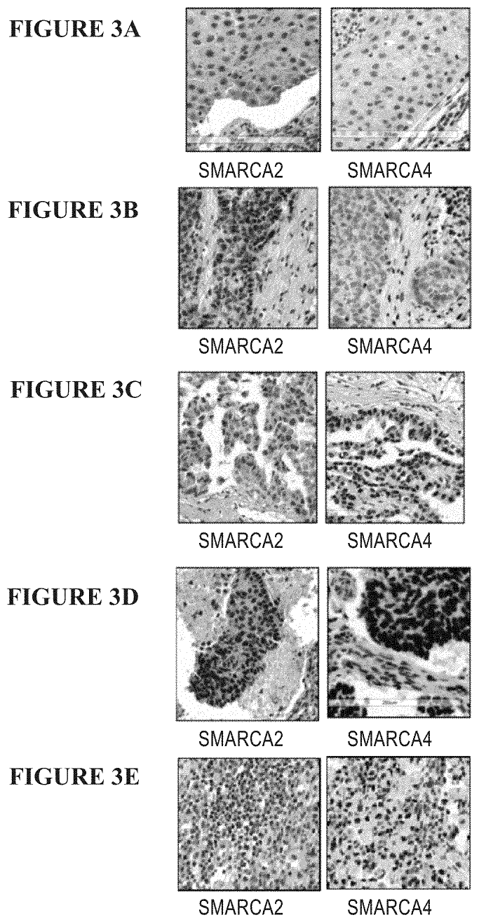

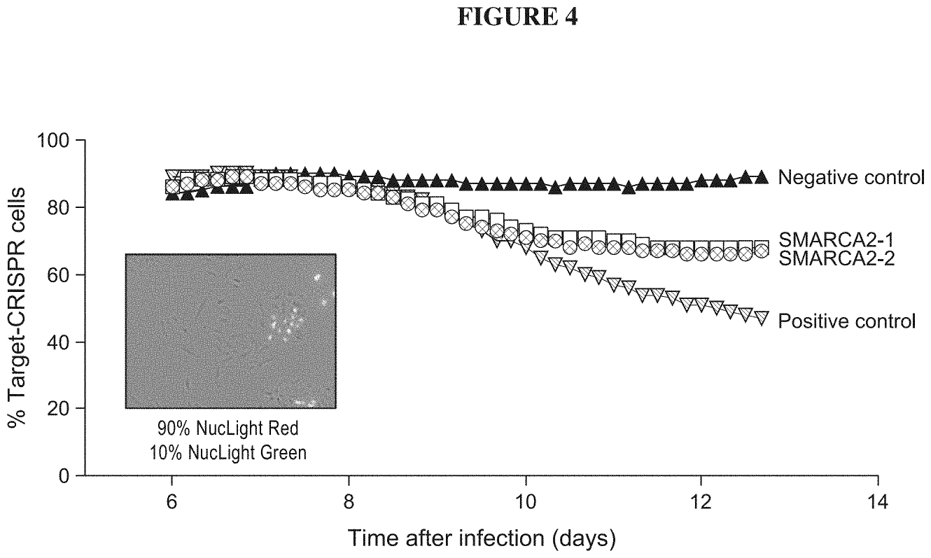

A panel of 226 non-small cell lung cancer tumor samples was screened for SMARCA2/4 protein expression via an immunohistochemistry (IHC) assay that was optimized for SMARCA2 and SMACRA4 detection. The IHC slides are shown in FIGS. 3A-3E. The results are summarized in Table 3.

TABLE 3
Frequencies of SMARCA2 and SMARCA4 loss in NSCLC tumor
samples.

| | SMARCA2 | |
|---|---|---|
| SMARCA4 | Negative | Positive |
| Negative | 3% (6) | 1% (2) |
| Positive | 35% (80) | 61% (138) |

### Example 3: Anti-Proliferative Effect of SMARCA2 Inhibition in Cells

The anti-proliferative effect of SMARCA2 knockout or inhibition in SMARCA4 mutant cell lines, suggested by the CRISPR pooled screen results described in Example 1, was further evaluated in 3 target validation assays: a genotype sequencing assay, a fluorescent competition assay, and CRISPR domain-centric screening.

The genotype sequencing assay (NGS) confirmed dependence of cell proliferation on SMARCA2 sensitivity. The fluorescent competition assay validated. Phenotypic validation of CRISPR pooled screen results is challenging for single genes due to strong selection for wild-type or non-functional mutations.

The CRISPR domain-centric screening demonstrated dependence of the anti-proliferative effect on the targeted catalytic domain. Specifically, inhibition of the SMARCA2 ATPase domain, was found to drive the anti-proliferative effect of SMARCA2 knockout in cells.

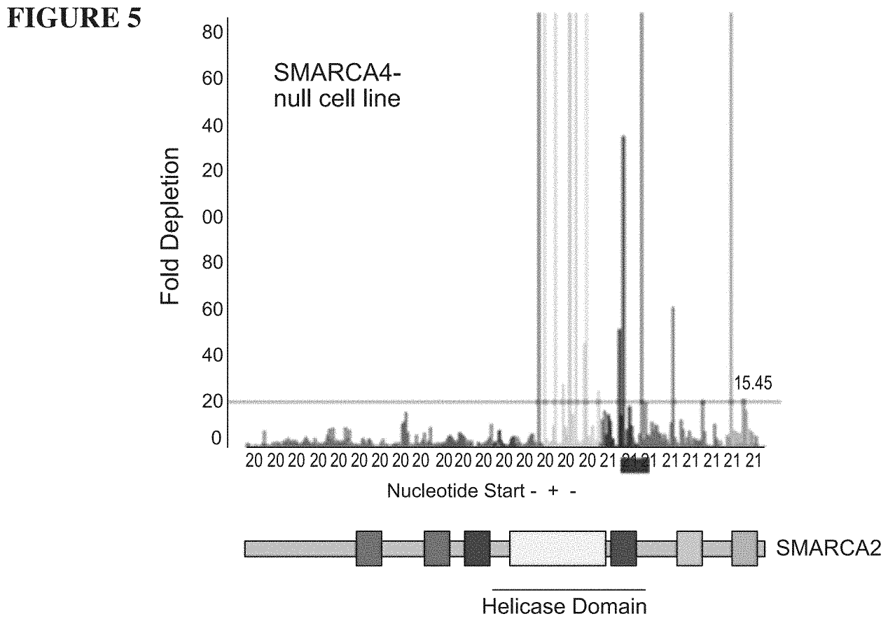

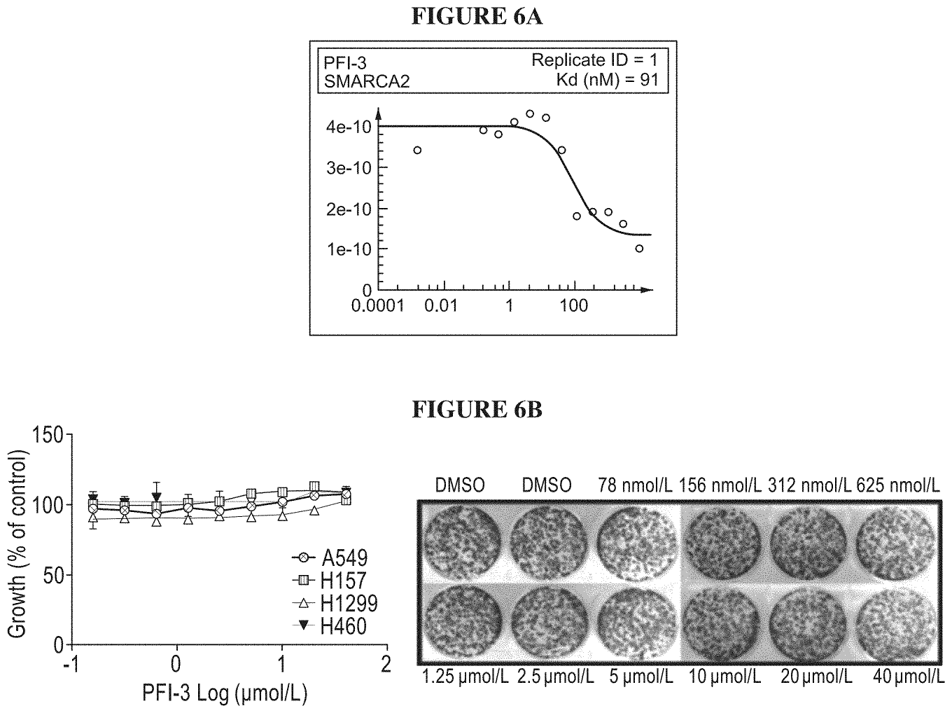

CRISPR guides targeting the SMARCA2 helicase domain were found to have the strongest anti-proliferative effect. Guides targeting the SMARCA2 bromodomain showed minimal effect (FIG. 5). Furthermore, treatment with the SMARCA2/4 bromodomain inhibitor PFI-3 has no functional effect on cell growth in SMARCA4 wild-type or mutant cell lines (FIGS. 6A-6C).

### Example 4: Screening for ATPase Inhibitors

Known inhibitors, targeting the bromodomain, were found to have no effect on SMARCA2 ATPase activity (See Table 4). ATPase and not bromodomain function of SMARCA2 is required for viability of SMARCA4 loss of function cells. As such, the development of viable antagonists or inhibitors (i.e., ATPase inhibitors) requires methods of monitoring ATPase activity.

TABLE 4
Inhibition of SMARCA2 with known bromodomain inhibitors

| | | | | |
|---|---|---|---|---|
| | BMCL 2968 | I-BET151 | JQ1 | PFI3 |
| | 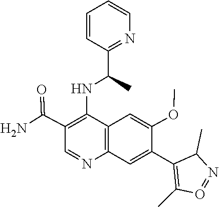 | 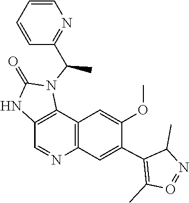 | 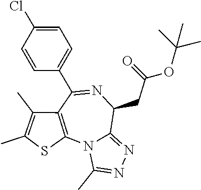 | 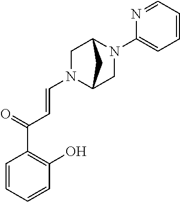 |
| % inh | 9.8 | -5.2, 15 | 4.9 | 5 |
| SMARCA2 | | | | |
| at 10 nM | | | | |

SMARCA2 is normally found in a multidomain complex. Hence, a first step in the screening and development of suitable compounds was to determine if the isolated full length protein behaves similar to the cellular system and if a suitable construct for biophysics could be produced.

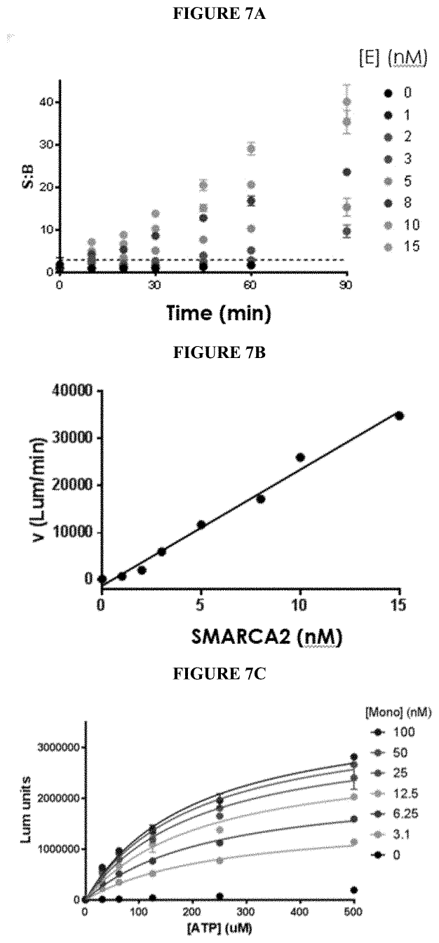

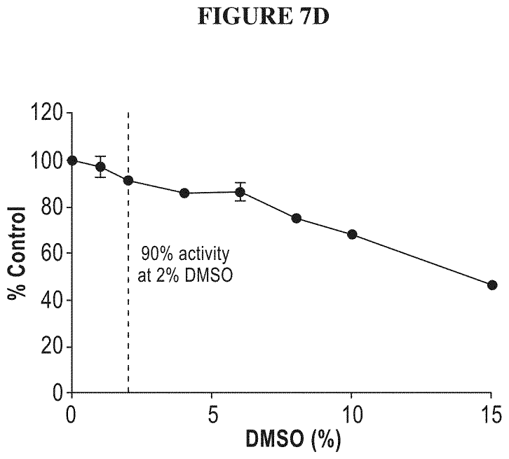

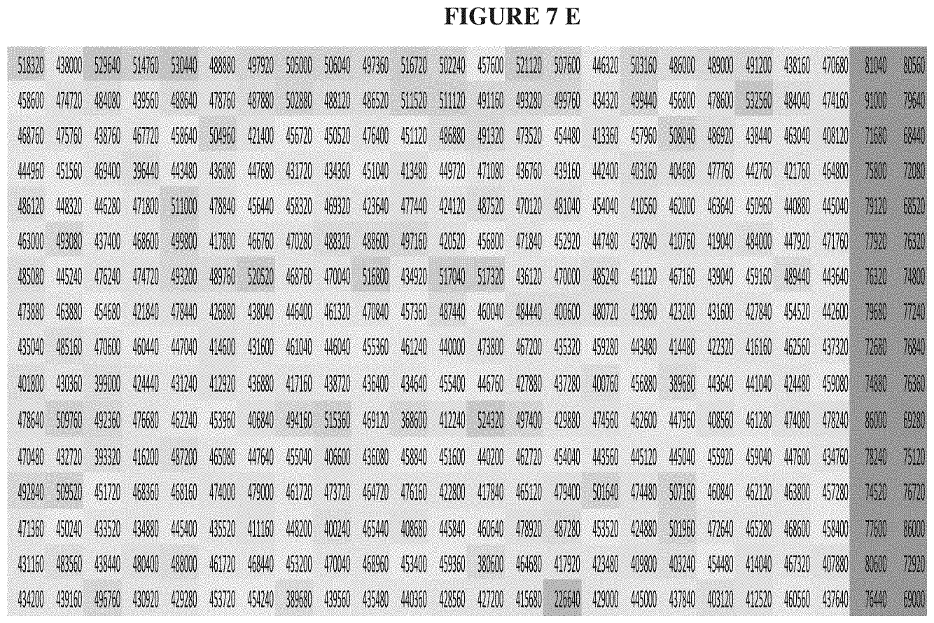

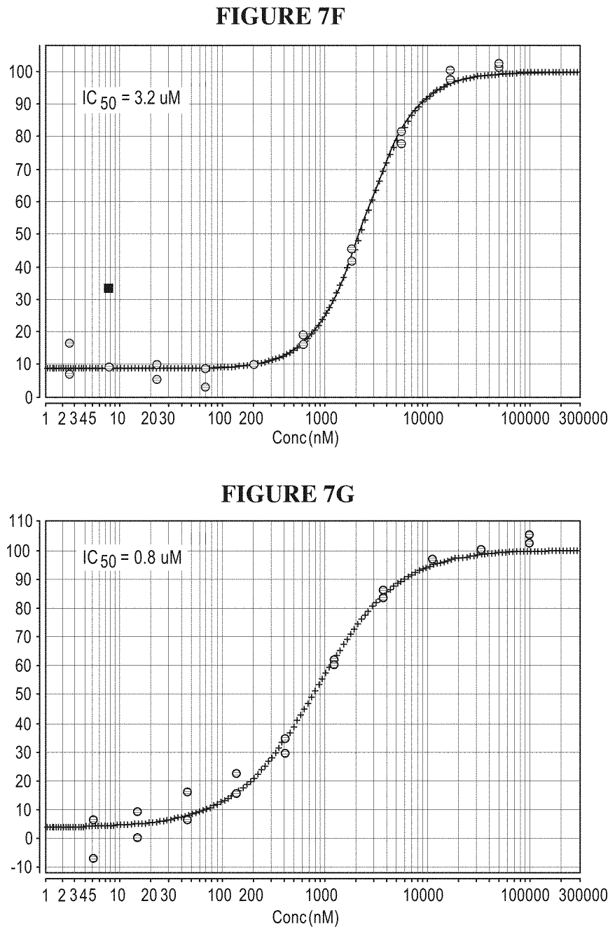

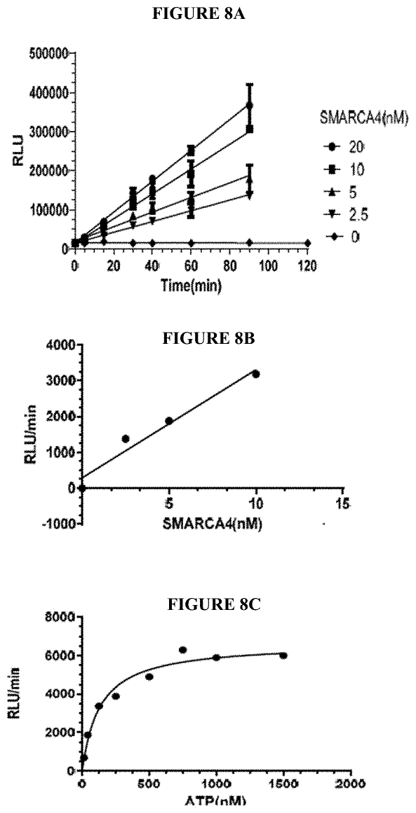

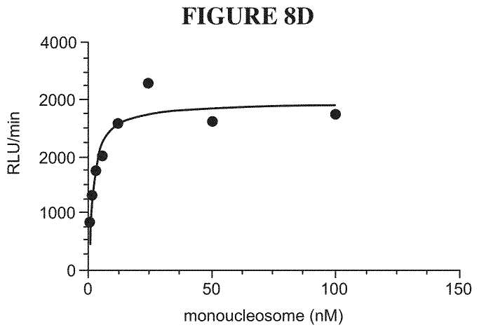

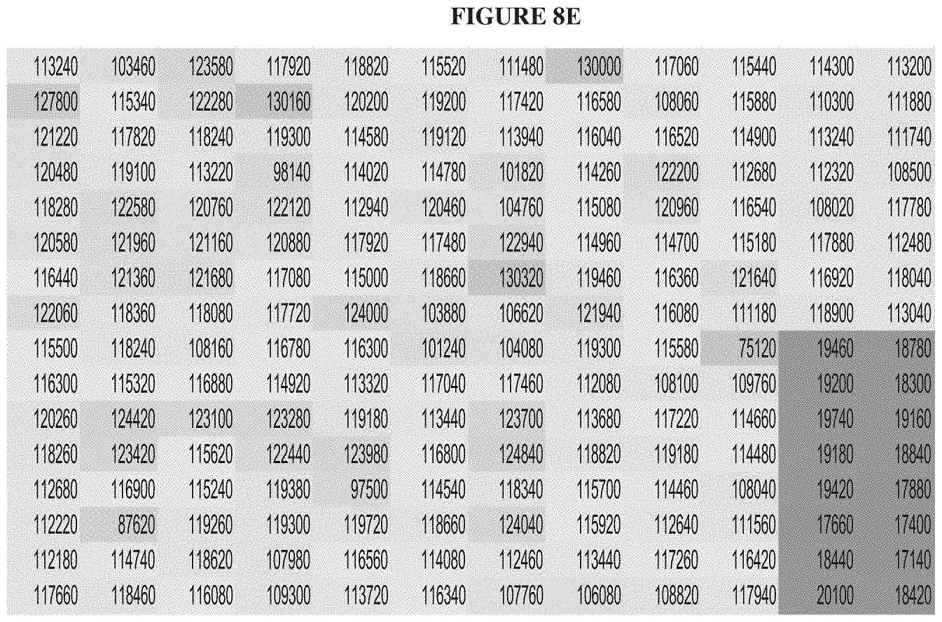

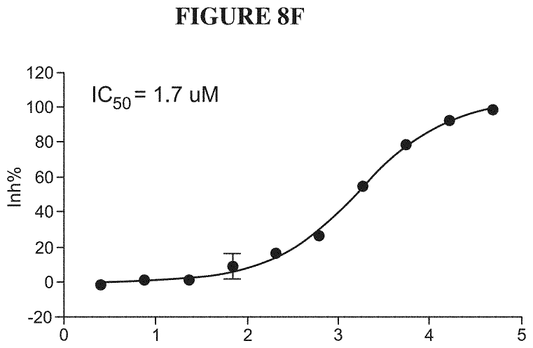

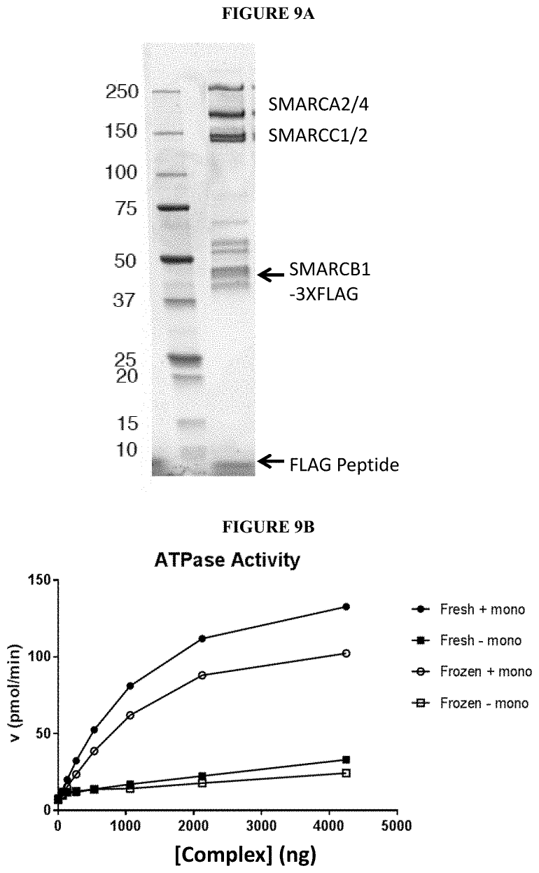

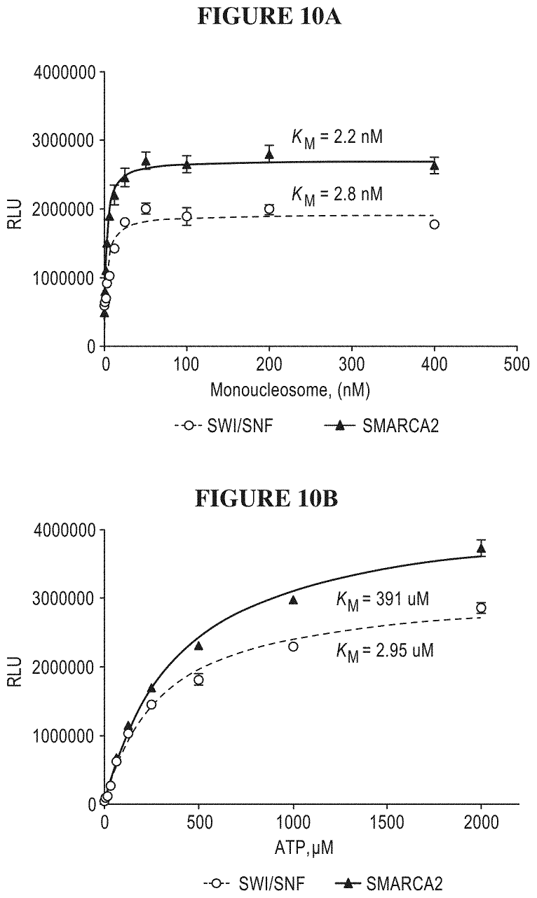

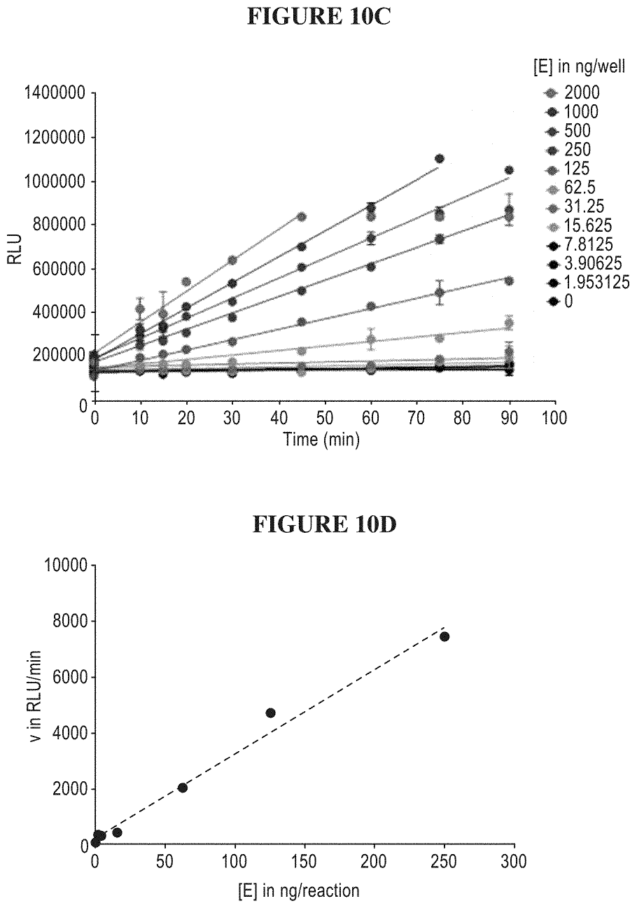

Isolated full length SMARCA2 (FL-SMARCA2) was found to be well behaved in activity assays. The signal to background ratio, ATPase activity as a function of protein concentration, and the dependence of ATP activity on mononucleosome substrate were determined for the isolated full length SMARCA2 and SMARACA4 in a high throughput screening bioluminescent assay (HTS ADP-Glo™ format). The results are summarized in FIGS. 7A-7C and FIGS. 8A-8C, respectively. The purified SWI/SNF complex demonstrated mononucleosome dependent ATPase activity in an ATPase assay. ATPase activity as a function of concentration for the purified complex was found exhibit a 16-18 fold higher slope in the presence of mononucleosome (FIG. 9B). The activity and dependence on nucleosome were shown to be similar between the isolated full length SMARCA2 and the SWI/SNF complex. In addition, the purified protein complex and isolated SMARCA2 demonstrated similar kinetic parameters (FIGS. 10A-10D). Consequently, isolated SMARCA2 was used for further development and a 474K HTS was completed.

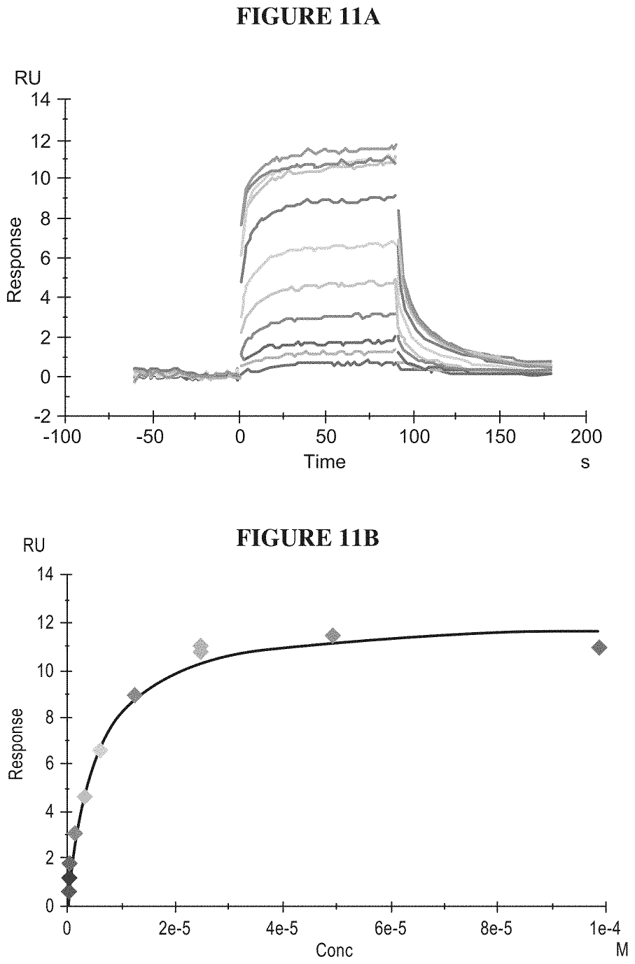

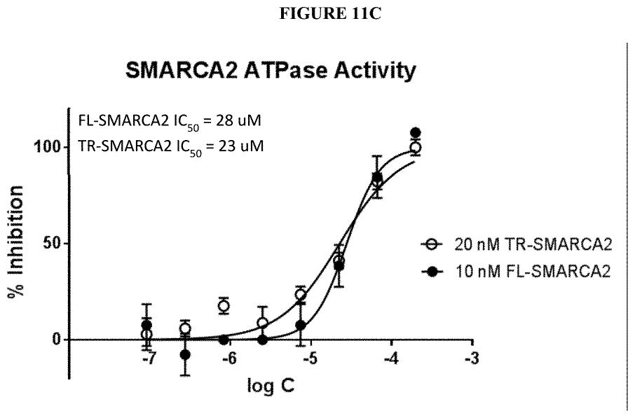

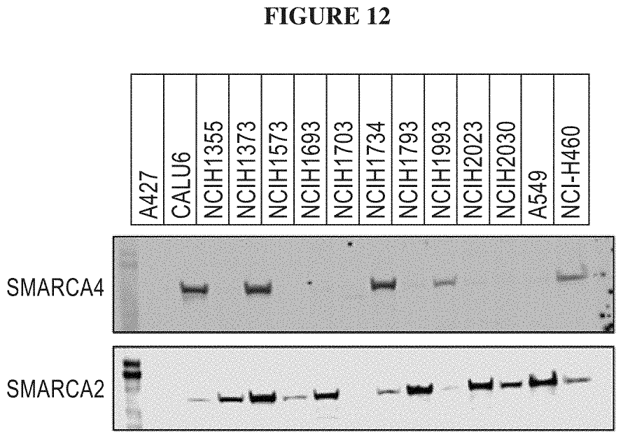

Hits from the HTS assay were further evaluated and prioritized for IC₅₀ and affinity interactions (SMARCA2 binding) determined in Surface Plasmon Resonance (SPR), wherein the IC₅₀ was determined in a 2-amino-6-mercapto-7-methylpurine ribonucleoside/Purine Nucleoside Phosphorylase (MESG/PNP) assay (FIG. 11).

Unless otherwise defined, all technical and scientific terms used herein have the same meaning as commonly understood by one of ordinary skill in the art to which this invention belongs. In the specification, the singular forms also include the plural unless the context clearly dictates otherwise. Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. Where names of cell lines or genes are used, abbreviations and names conform to the nomenclature of the American Type Culture Collection (ATCC) or the National Center for Biotechnology Information (NCBI), unless otherwise noted or evident from the context.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects are excluded are not set forth explicitly herein.

What is claimed is:

1. A method of identifying at least one SMARCA2 inhibitor, the method comprising:
a) incubating a first plurality of isolated SMARCA2 proteins under conditions that comprise a plurality of mononucleosomes and ATP;
b) incubating a second plurality of isolated SMARCA2 proteins under conditions that comprise a plurality of mononucleosomes, ATP and at least one candidate compound;
c) determining the ATPase activity of the first plurality of isolated SMARCA2 proteins incubated in step (a);
d) determining the ATPase activity of the second plurality of isolated SMARCA2 proteins incubated in step (b);
e) comparing the ATPase activity of the first plurality of isolated SMARCA2 proteins and the ATPase activity of the second plurality of isolated SMARCA2 proteins, wherein the candidate compound is identified as a SMARCA2 inhibitor when the ATPase activity of the second plurality of isolated SMARCA2 proteins is less than the ATPase activity of the first plurality of isolated SMARCA2 proteins.

2. The method of claim 1, wherein the isolated SMARCA2 proteins are isolated full-length SMARCA2 proteins.

3. The method of claim 1, wherein the isolated SMARCA2 proteins are isolated truncated SMARCA2 proteins.

4. The method of claim 1, wherein determining the ATPase activity steps (c) and (d) comprise the use of a bioluminescence assay.

5. The method of claim 1, wherein the candidate is identified as a SMARCA2 inhibitor when the ATPase activity of the second plurality of isolated SMARCA2 proteins is at least 5-fold less than the ATPase activity of the first plurality of isolated SMARCA2 proteins.

6. The method of claim 4, wherein the candidate is identified as a SMARCA2 inhibitor when the ATPase activity of the second plurality of isolated SMARCA2 proteins is at least 10-fold less than the ATPase activity of the first plurality of isolated SMARCA2 proteins.

7. The method of claim 6, wherein the candidate is identified as a SMARCA2 inhibitor when the ATPase activity of the second plurality of isolated SMARCA2 proteins is at least 100-fold less than the ATPase activity of the first plurality of isolated SMARCA2 proteins.

8. The method of claim 1, wherein steps (a)-(d) are performed concurrently.

9. The method of claim 1, further comprising:
performing steps (b) and (d) at different concentrations of the at least one candidate compound; and
determining a SMARCA2 IC₅₀ for the at least one candidate compound based on the ATPase activity measured at the different concentrations.

10. A method of identifying at least one selective SMARCA2 inhibitor, the method comprising:
a) incubating a first plurality of isolated SMARCA2 proteins under conditions that comprise a plurality of mononucleosomes and ATP;
b) incubating a second plurality of isolated SMARCA2 proteins under conditions that comprise a plurality of mononucleosomes, ATP and at least one candidate compound;
c) incubating a first plurality of isolated SMARCA4 proteins under conditions that comprise a plurality of mononucleosomes and ATP;
d) incubating a second plurality of isolated SMARCA4 proteins under conditions that comprise a plurality of mononucleosomes, ATP and the at least one candidate compound;
e) determining the ATPase activity of the first plurality of isolated SMARCA2 proteins incubated in step (a);
f) determining the ATPase activity of the second plurality of isolated SMARCA2 proteins incubated in step (b);
g) determining the ATPase activity of the first plurality of isolated SMARCA4 proteins incubated in step (c);
h) determining the ATPase activity of the second plurality of isolated SMARCA4 proteins incubated in step (d);
i) comparing the ATPase activity of the first plurality of isolated SMARCA2 proteins and the ATPase activity of the second plurality of isolated SMARCA2 proteins;
j) comparing the ATPase activity of the first plurality of isolated SMARCA4 proteins and the ATPase activity of the second plurality of isolated SMARCA4 proteins; and
k) determining that the at least one candidate compound is identified as a selective SMARCA2 inhibitor when the ATPase activity of the second plurality of isolated SMARCA2 proteins is less than the ATPase activity of the first plurality of isolated SMARCA2 proteins and:
i) the ATPase activity of the second plurality of isolated SMARCA4 proteins and the ATPase activity of the first plurality of isolated SMARCA4 proteins is the same;
ii) the ATPase activity of the second plurality of isolated SMARCA4 proteins is greater than the ATPase activity of the first plurality of isolated SMARCA4 proteins; or
iii) the decrease in ATPase activity of the second plurality of isolated SMARCA2 proteins as compared to the ATPase activity of the first plurality of isolated SMARCA2 is a greater than a decrease in ATPase activity of the second plurality of isolated SMARCA4 proteins as compared to the ATPase activity of the first plurality of isolated SMARCA4 proteins.

11. The method of claim 10, further comprising:
performing steps (b), (d), (f) and (h) at different concentrations of the at least one candidate compound;
determining a SMARCA2 IC₅₀ and a SMARCA4 IC₅₀ for the at least one candidate compound based on the ATPase activity measured at the different concentrations, wherein the at least one candidate compound is identified as a selective SMARCA2 inhibitor when its SMARCA2 IC₅₀ is at least 40% lower than its SMARCA4 IC₅₀.

12. The method of claim 11, wherein the at least one candidate compound is identified as a selective SMARCA2 inhibitor when its SMARCA2 IC₅₀ is at least 90% lower than its SMARCA4 IC₅₀.

13. The method of claim 11, wherein the isolated SMARCA2 proteins are isolated full-length SMARCA2 proteins and the isolated SMARCA4 proteins are isolated full-length SMARCA4 proteins.

14. The method of claim 11, wherein the isolated SMARCA2 proteins are isolated truncated SMARCA2 proteins and the isolated SMARCA4 proteins are isolated truncated SMARCA4 proteins.

15. The method of claim 11, wherein determining the ATPase activity steps (e)-(h) comprise the use of a bioluminescence assay.

16. The method of claim 11, wherein steps (a)-(h) are performed concurrently.

17. A method of treating cancer in a subject in need thereof, comprising administering to the subject a compound identified as a SMARCA2 inhibitor according to the method of claim 1.

18. The method of claim 17, wherein the subject or cell of the subject exhibits a decreased activity or function of SMARCA4 when compared to a control level of the activity or the function of SMARCA4

19. A method of treating cancer in a subject in need thereof, comprising administering to the subject a compound identified as a SMARCA2 inhibitor according to the method of claim 11.

20. The method of claim 19, wherein the subject or cell of the subject exhibits a decreased activity or function of SMARCA4 when compared to a control level of the activity or the function of SMARCA4.
